(19) United States
(12) Patent Application Publication
(10) Pub. No.: US 2011/0077279 A1
(43) Pub. Date: Mar. 31, 2011

(54) ANGIOTENSIN II RECEPTOR ANTAGONISTS

(76) Inventors: Nicoletta Almirante, Milano (IT); Massimiliano Ferrario, Ceriano Laghetto (Milano ) (IT); Silvia Stefanini, San Donato Milanese (Milano ) (IT); Stefano Biondi, Pero (Milano ) (IT); Ennio Ongini, Segrate (Milano ) (IT)

(21) Appl. No.: 12/995,640

(22) PCT Filed: May 18, 2009
(86) PCT No.: PCT/EP09/55985

§ 371 Date: Dec. 1, 2010

Related U.S. Application Data
(60) Provisional application No. 61/059,994, filed on Jun. 9, 2008.

(51) Int. Cl. A61K 31/4184; C07D 493/04; A61P 9/12

(52) U.S. Cl. 514/382; 548/254

(57) ABSTRACT

A compound having the structure

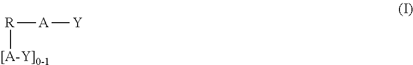

wherein R is an angiotensin II receptor antagonist selected from the group consisting of (IIa)-(IIh);
A is

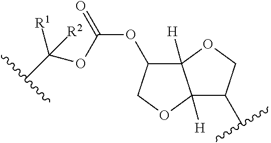

wherein R¹ and R² are independently selected from the group consisting of hydrogen and C₁₋₄ alkyl.

Y is X⁰—Z wherein X⁰ is selected from the group consisting of:
—O—, —O—CO—, —OCOO—, —OCONH— and —OSO₂—;
Z is a nitric oxide releasing moiety,
or a pharmaceutically acceptable salt thereof.

### BACKGROUND OF THE INVENTION

U.S. Pat. No. 5,138,069 generically and specifically describes 2-butyl-4-chloro-1-[p-(o-1H-tetrazol-5-ylphenyl)-benzyl]imidazole-5-methanol potassium salt and 2-butyl-4-chloro-1-[(2′-1H-tetrazol-5-yl)biphenyl-4-yl)methyl]imidazole-5-carboxylic acid. Columns 261-263 of U.S. Pat. No. 5,136,069 describe general procedures for formulating compounds described in the patent, including capsules, tablets, injection formulations, and suspensions. U.S. Pat. No. 5,153,197, describes the use of these compounds, alone and in combination with a diuretic, to treat a patient having hypertension.

WO2005011646 describes angiotensin II receptor blocker nitroderivatives, pharmaceutical compositions containing them and their use for the treatment of cardiovascular, renal and chronic liver diseases, inflammatory processes and metabolic syndromes. The publication describes a variety of angiotensin receptor blocker compounds each of which are covalently linked in a variety of ways to a nitric oxide group. Specific examples include angiotensin receptor blockers with one covalently-linked nitric oxide group, and angiotensin receptor blockers with two independently-covalently-linked nitric oxide groups. WO2005023182 describes nitrosated and nitrosylated cardiovascular compounds, and compositions comprising at least one nitrosated and nitrosylated cardiovascular compound and optionally at least one nitric oxide donor. The cardiovascular compound which is nitrosated or nitrosylated may be an aldosterone antagonist, an angiotensin II receptor antagonist, a calcium channel blocker, an endothelin antagonist, a hydralazine compound, a neutral endopeptidase inhibitor or a renin inhibitor. The nitric oxide donor may be selected from S-nitrosothiols, nitrites, nitrates, N-oxo-N-nitrosamines, furoxans, and sydnonimines.

WO2005070868 describes combination therapy for treating cyclooxygenase-2 mediated diseases or conditions at risk of thrombotic cardiovascular events which involves administering selected cyclooxygenase-2 inhibitor in combination with a nitric oxide donating compound such as 5,6-bis(nitrooxy)hexyl acetate, 6-hydroxyhexane-1,2-diyl dinitrate, 5-hydroxypentane-1,2-diyl dinitrate, (5R)-5,6-bis(nitrooxy)hexyl 4-nitrobenzoate, (5S)-5,6-bis(nitrooxy)hexyl 4-nitrobenzoate, (2R)-6-hydroxyhexane-1,2-diyl dinitrate, (2S)-6-hydroxyhexane-1,2-diyl dinitrate, (2S)-propane-1,2-diyl dinitrate, and (2R)-propane-1,2-diyl dinitrate.

### SUMMARY OF THE INVENTION

The present invention includes angiotensin II receptor antagonist nitrooxyderivatives, including 2-butyl-4-chloro-1-[(2′-(1-H-tetrazol-5-yl)biphenyl-4-yl)methyl]-imidazole-5-carboxylate nitrooxyderivatives, including various pharmaceutically acceptable salts and hydrates of these forms, and pharmaceutical formulations for controlled and sustained delivery of these forms to a patient.

The salts include non-toxic salts such as those derived from inorganic acids, e.g. hydrochloric, hydrobromoic, sulfuric, sulfamic, phosphoric, nitric and the like, or the quaternary ammonium salts which are formed, e.g., from inorganic or organic acids or bases. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The invention also includes a method for treating hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, by administering an angiotensin II receptor antagonist of the invention to a patient having one or more of these conditions.

### DETAILED DESCRIPTION OF THE INVENTION AND PREFERRED EMBODIMENTS

Compounds of the invention are angiotensin II receptor antagonist nitrooxyderivatives having the general formula (I):

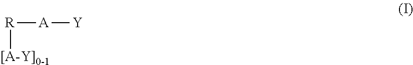

wherein R is selected from the group consisting of (IIa)-(IIh):

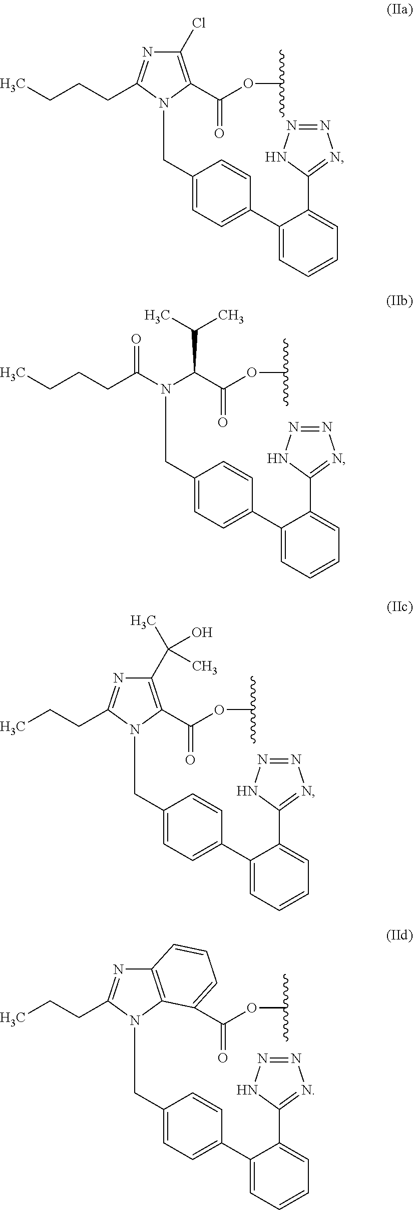

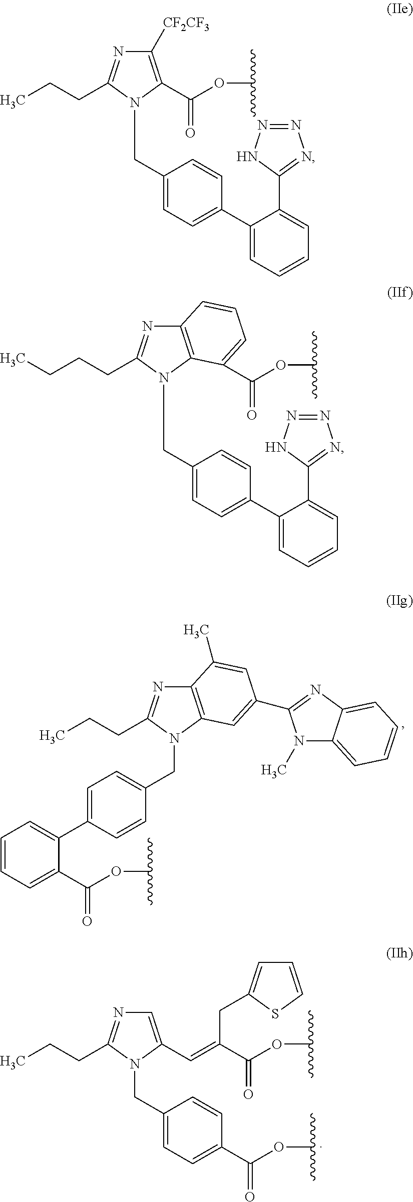

### A is

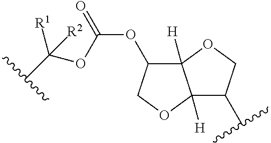

wherein R¹ and R² are independently selected from the group consisting of hydrogen and C₁₋₄ alkyl.

Y is X⁰—Z wherein X⁰ is selected from the group consisting of:

### —O—, —O—CO—, —OCOO—, —OCONH— and —OSO₂—;

Z is selected from the group consisting of:

### 1) R⁰—CH₂—ONO₂,

wherein R⁰ is a straight or branched C₁-C₁₀ alkylene;
2) (CH₂)ₙR³,
3) (CH₂)ₙ—O—CH₂—R³,
wherein

### R³ is —CH(ONO₂)R⁴;

R⁴ is —CH₃ or C₁₋₄ alkyl;
n is an integer from 1 to 6;

### 4) Y¹—R⁵,

wherein

### R⁵ is —CH(ONO₂)CH(ONO₂)R⁶;

R⁶ is selected from —CH₃, —CH₂CH₃ and —CH(CH₃)₂;
Y¹ is —(CH₂)₁₋₄—(X)₀₋₁—(CH₂)₀₋₄, wherein X is —O— or —CR⁷R⁸—; and R⁷ and R⁸ are independently selected from the group consisting of hydrogen and C₁-C₄ alkyl;

### 5) Y¹—CH(ONO₂)CH₂(ONO₂)

wherein Y¹ is as above defined;
6)

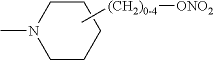

In another embodiment, A is selected from the group consisting of:

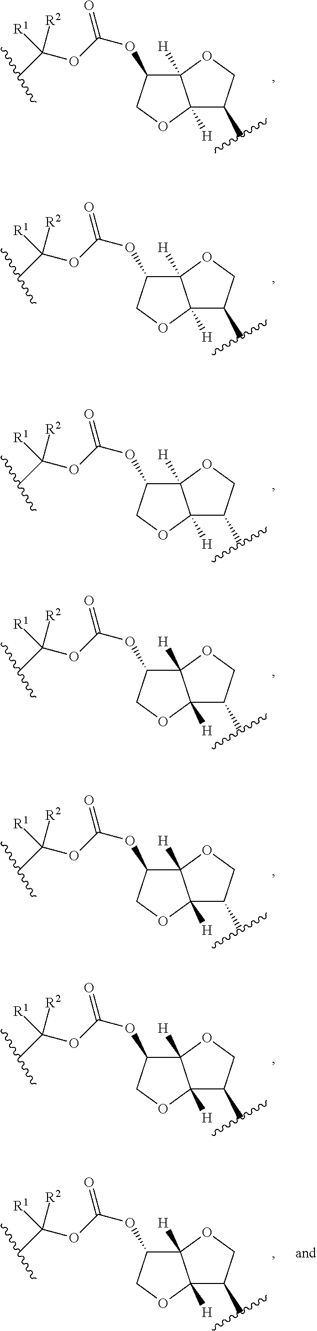

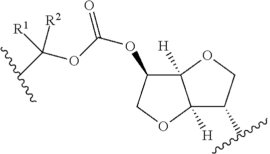

and all other variables are as previously defined.

In another embodiment, A is selected from the group consisting of:

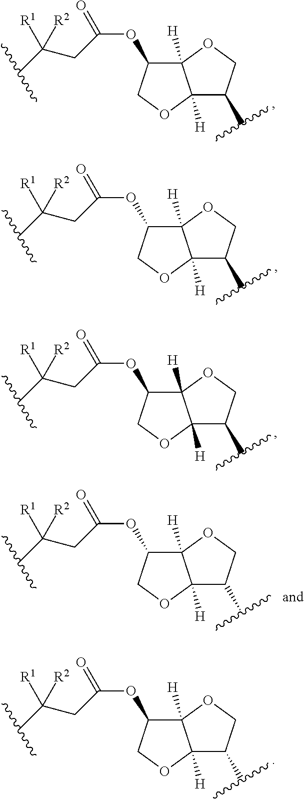

wherein R¹ and R², are independently selected from the group consisting of hydrogen and CH₃.

In one embodiment A is selected from the group consisting of:

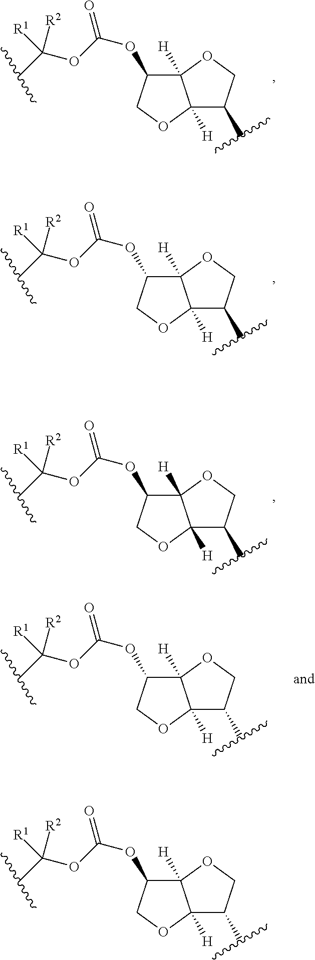

wherein R¹ is CH₃ and R² is H or CH₃.

In another embodiment, R is selected from the group consisting of (IIb), (IIc) and (IIh):

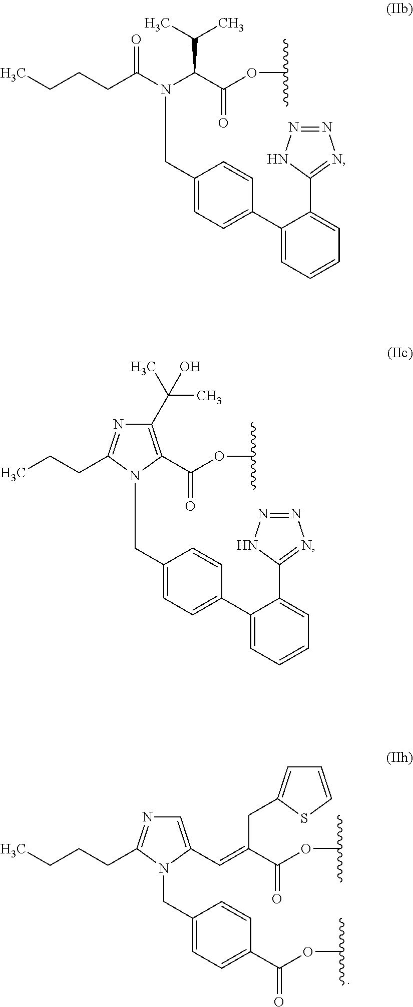

In another embodiment, R is selected from the group consisting of (IIa), (IId) and (IIg):

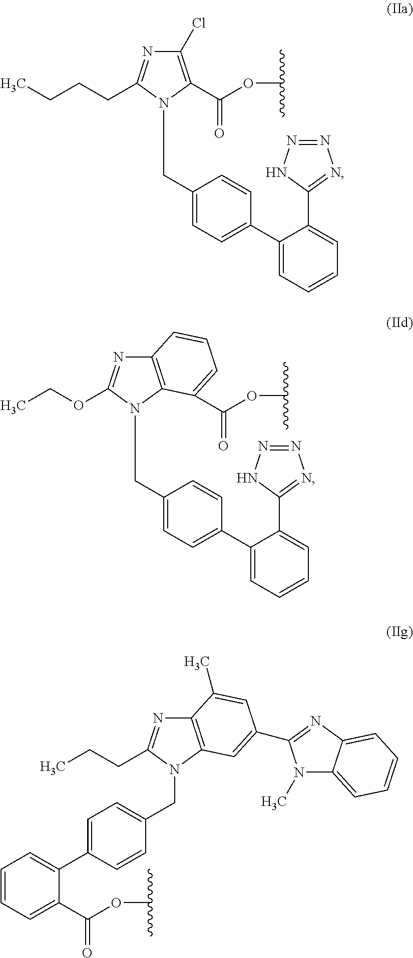

and all other variables are as previously defined.

In another embodiment; R is (IId):

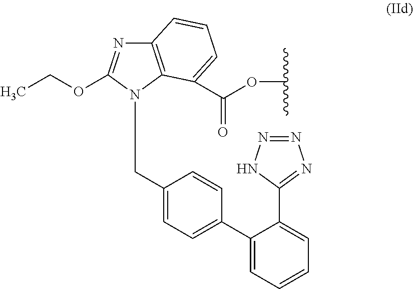

and A is selected from the group consisting of:

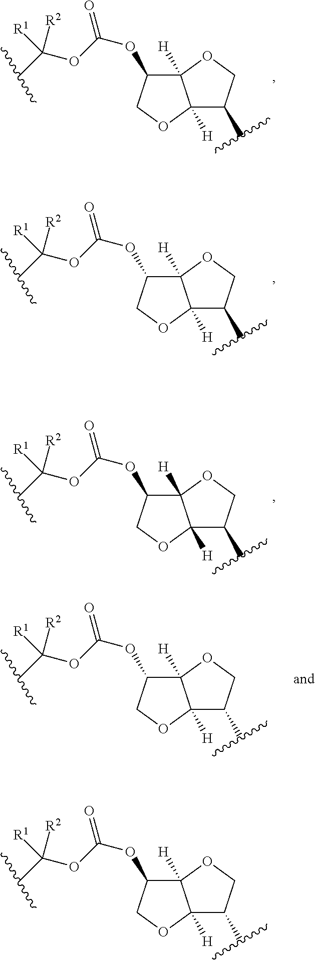

wherein R¹ and R² are CH₃.

In another embodiment, R is (IIa), (IId) or (IIg):

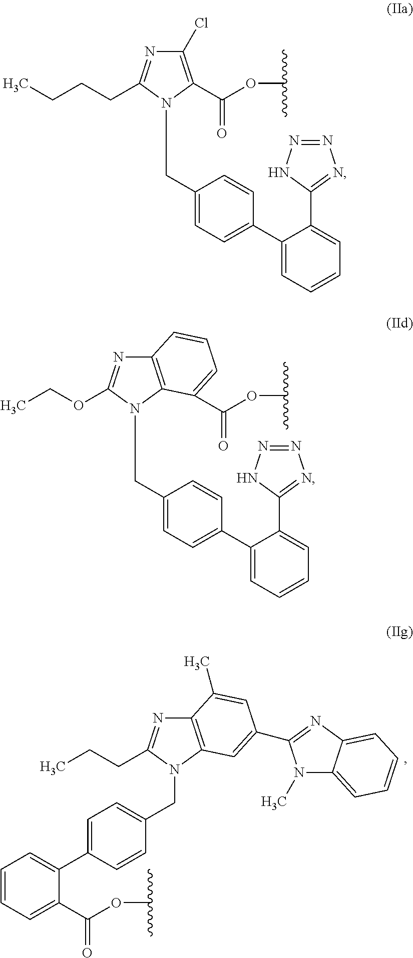

and A is selected from the group consisting of:

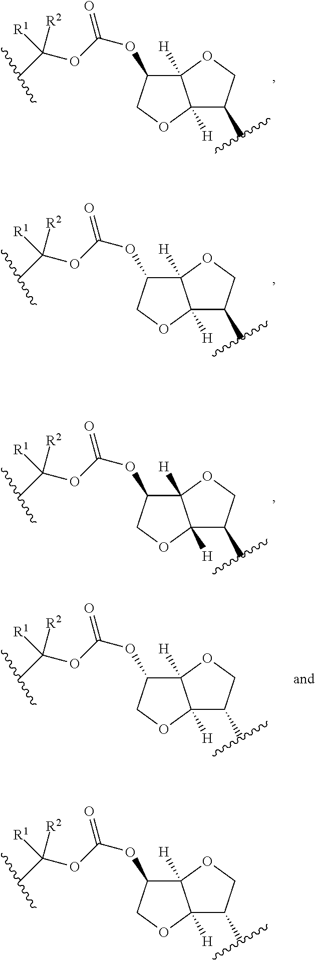

wherein R¹ is H and R² is CH₃.

In another embodiment, R is selected from (IIb) or (IIc):

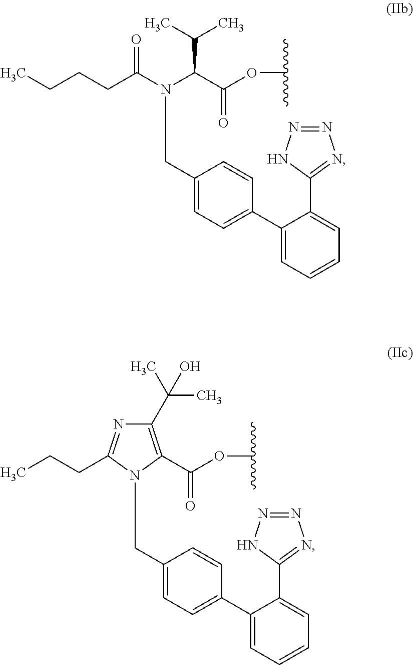

and A is selected from the group consisting of:

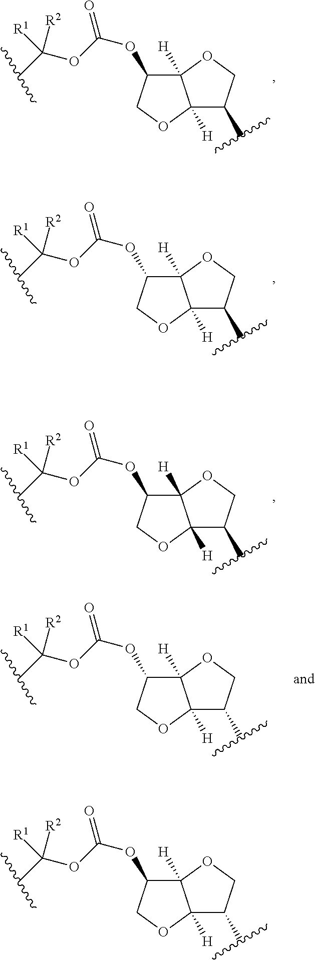

wherein R¹ is H and R² is CH₃.

In another embodiment, Y is selected from the group consisting of X⁰—Z wherein X⁰ is —O— and Z is selected from the group consisting of:

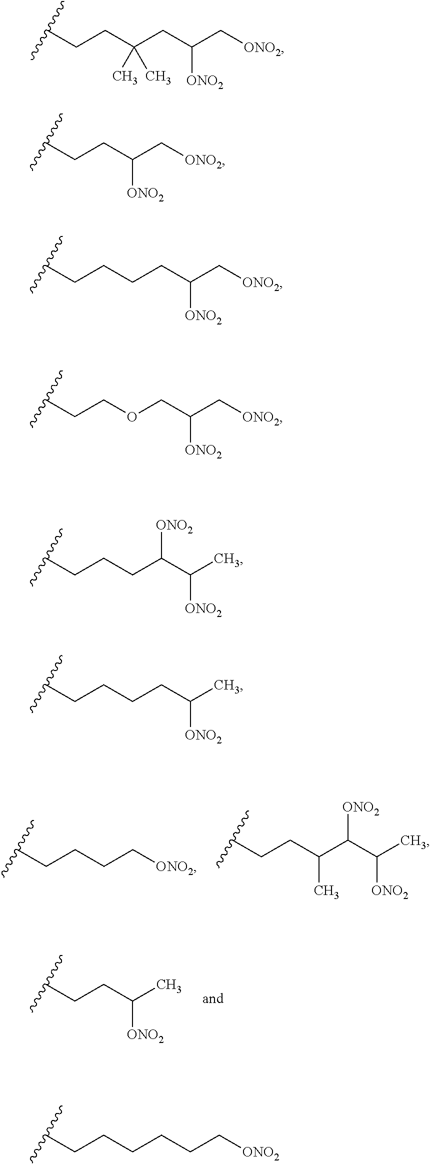

and all other stereoisomer thereof.

In another embodiment, Y is selected from the group consisting of X⁰—Z wherein X⁰ is —OCO— and Z is selected from the group consisting of:

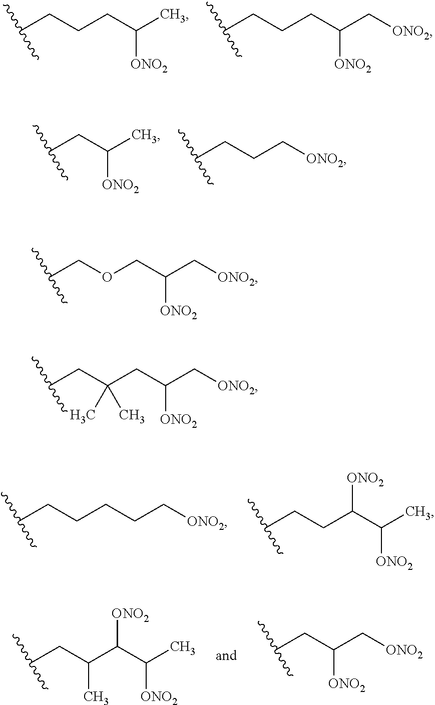

and all stereoisomer thereof.

In another embodiment, Y is selected from the group consisting of X⁰—Z wherein X⁰ is —OCOO— and Z is selected from the group consisting of:

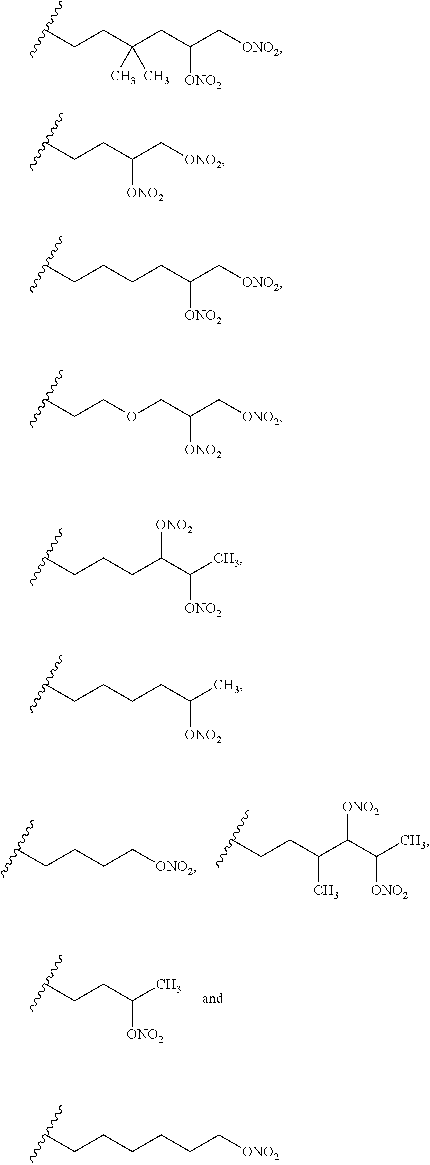

and all other stereoisomer thereof.

In another embodiment, Y is selected from the group consisting of X⁰—Z wherein X⁰ is —OCONH— and Z is selected from the group consisting of:

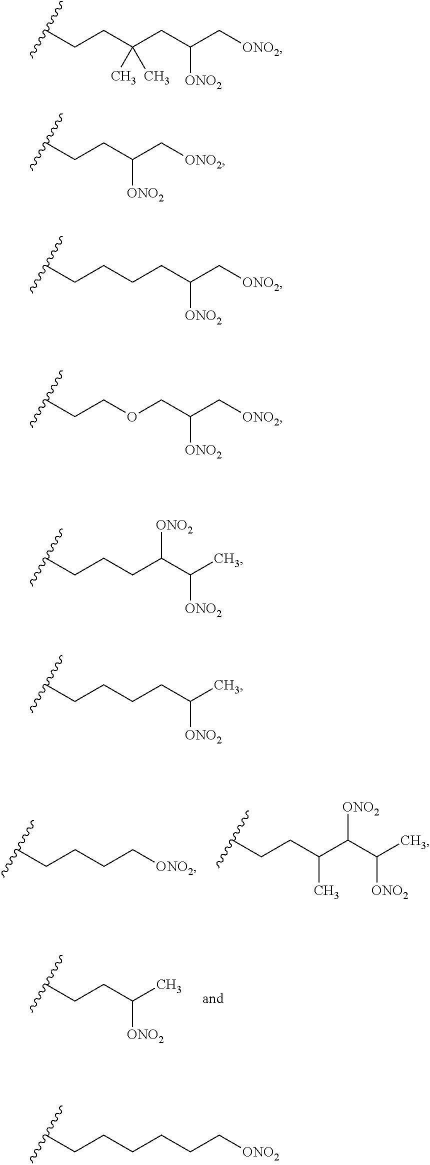

and all other stereoisomer thereof.

In another embodiment, the compound is selected from the group of compounds in Tables 1-20 shown below:

TABLE 1

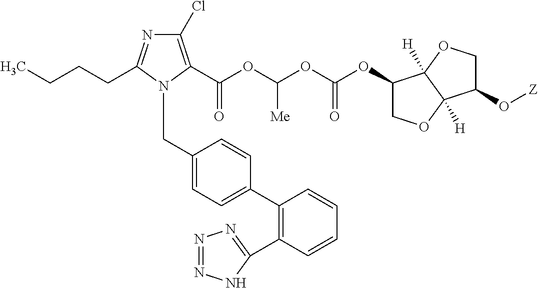

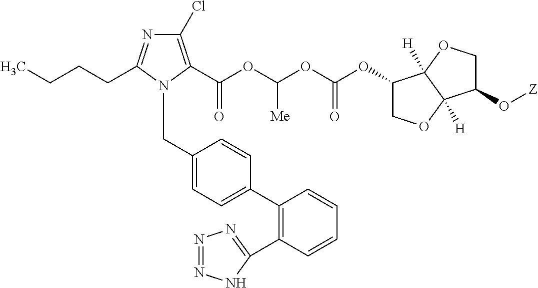

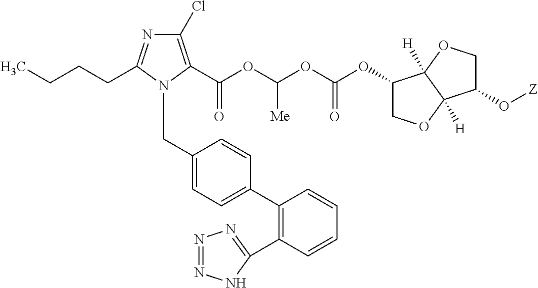

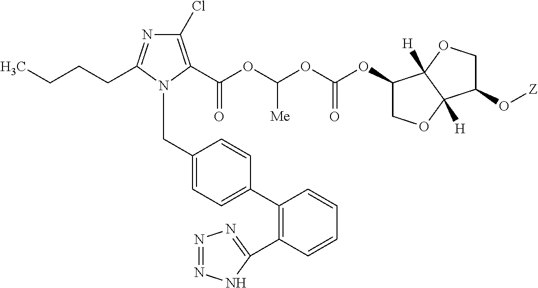

Z

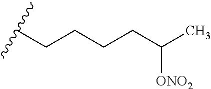

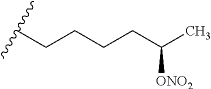

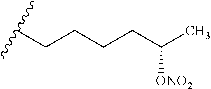

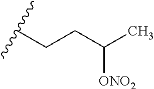

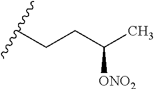

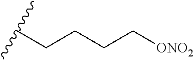

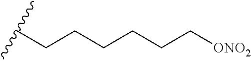

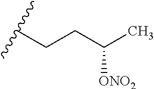

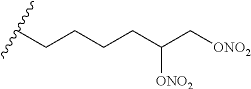

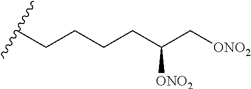

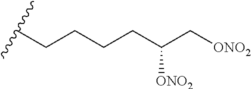

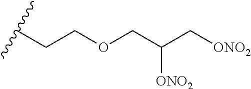

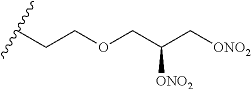

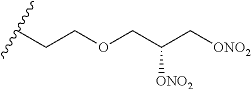

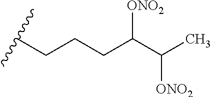

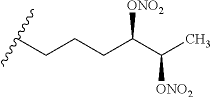

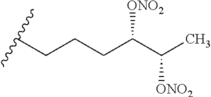

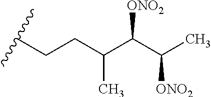

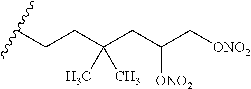

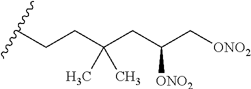

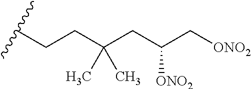

TABLE 2

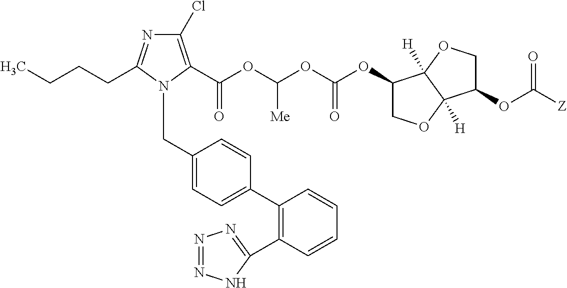

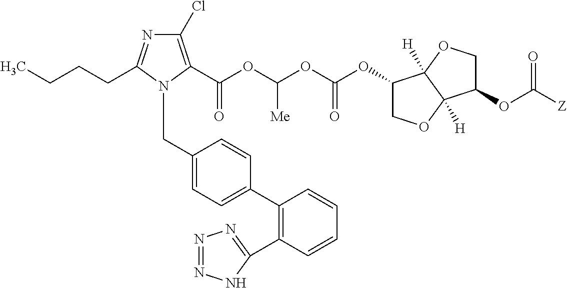

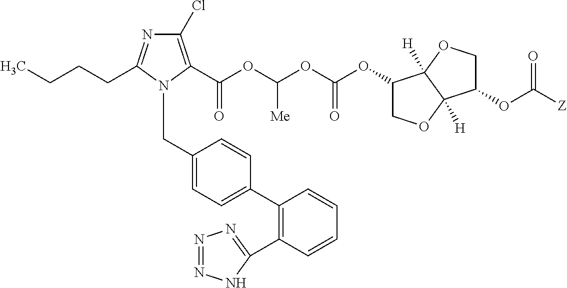

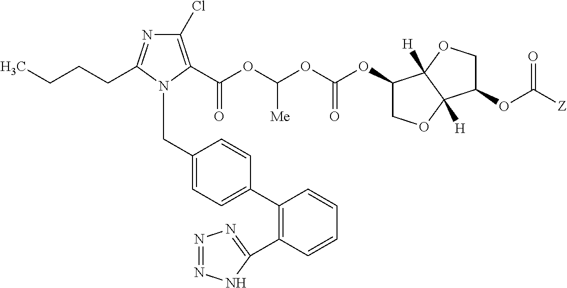

Z

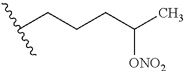

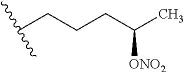

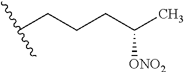

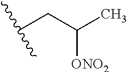

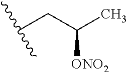

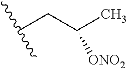

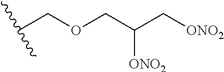

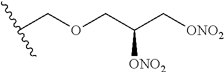

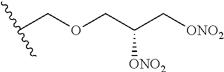

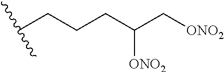

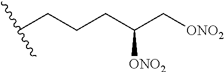

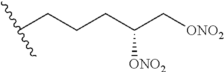

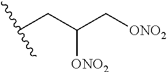

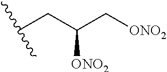

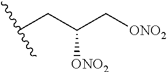

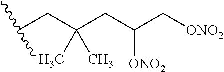

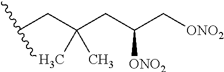

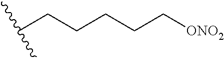

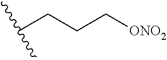

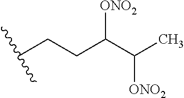

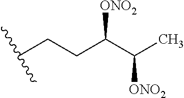

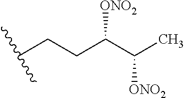

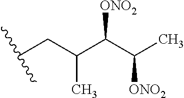

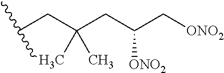

TABLE 3

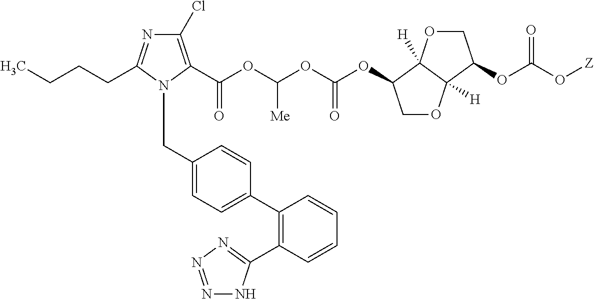

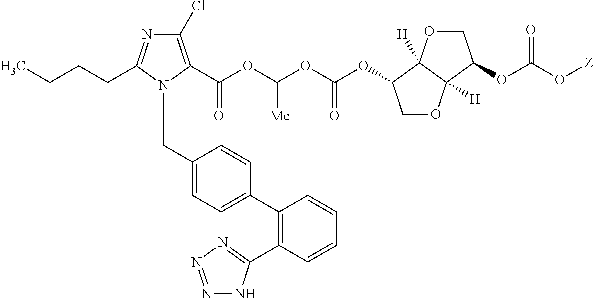

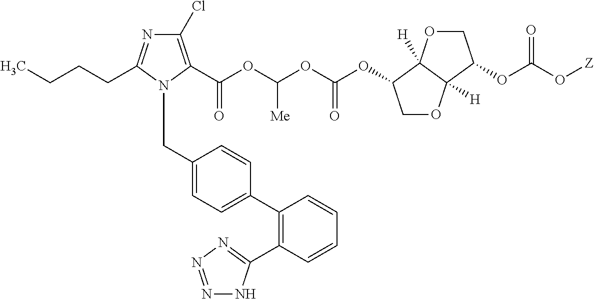

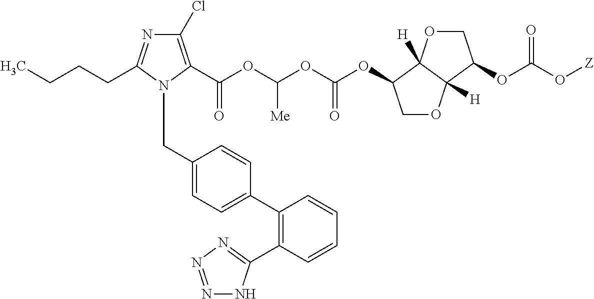

| Z | Z | Z |
|---|---|---|
| 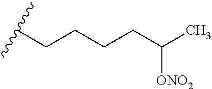 | 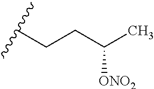 | 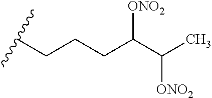 |
| 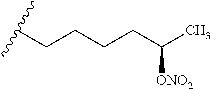 | 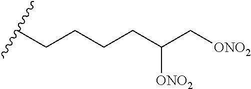 | 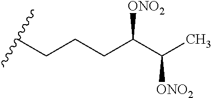 |
| 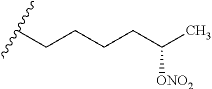 | 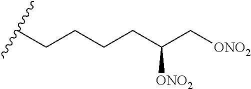 | 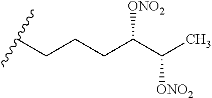 |
| 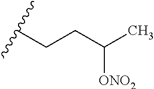 | 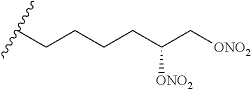 | 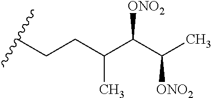 |
| 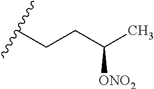 | 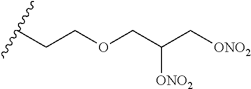 | 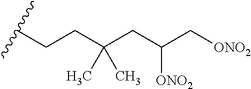 |
| 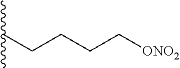 | 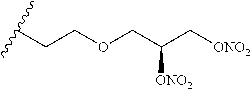 | 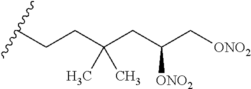 |
| 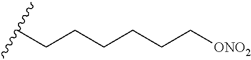 | 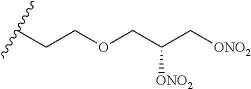 | 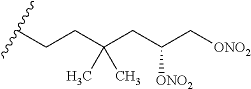 |

TABLE 4

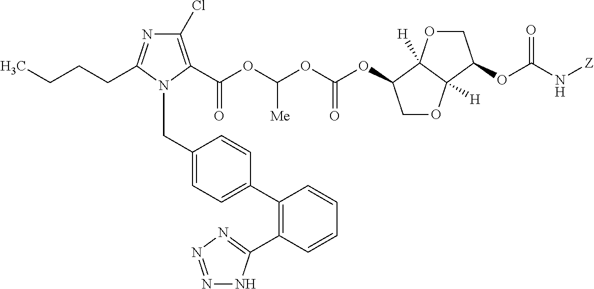

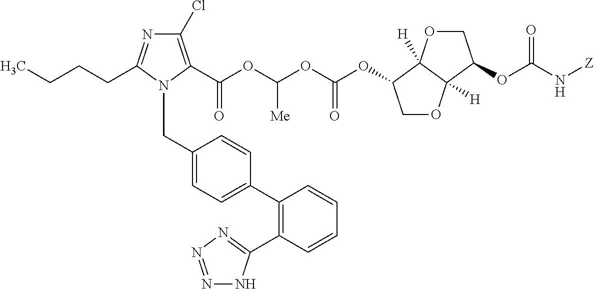

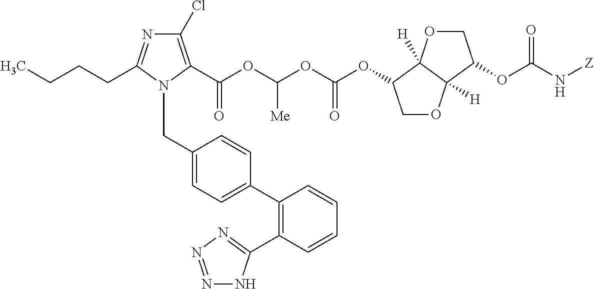

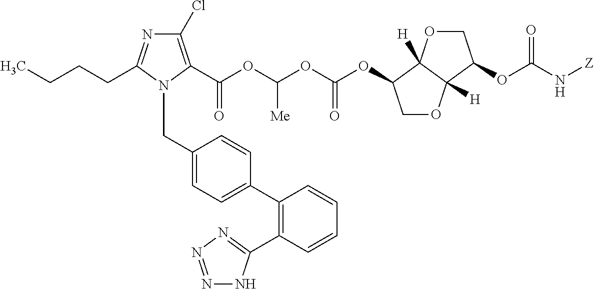

| Z | Z | Z |
|---|---|---|
| 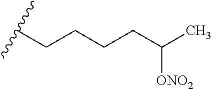 | 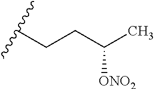 | 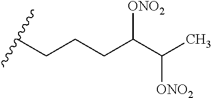 |
| 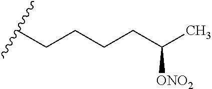 | 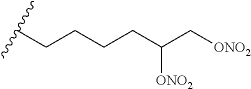 | 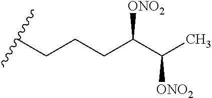 |
| 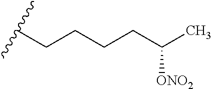 | 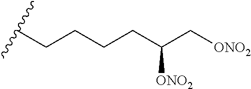 | 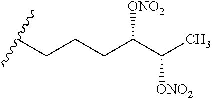 |
| 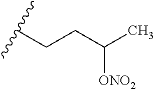 | 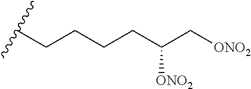 | 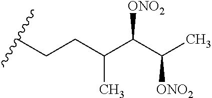 |
| 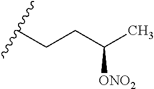 | 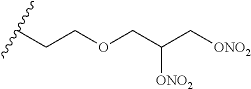 | 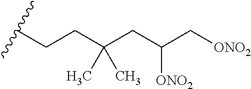 |
| 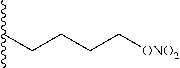 | 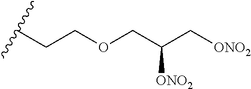 | 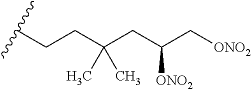 |
| 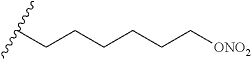 | 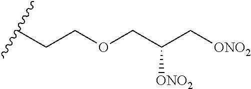 | 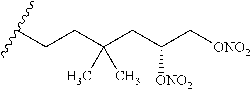 |

TABLE 5

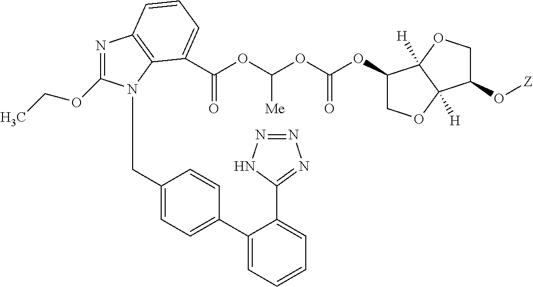

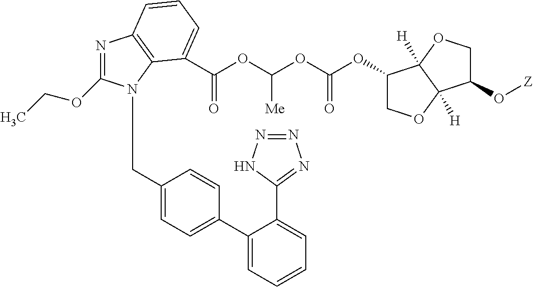

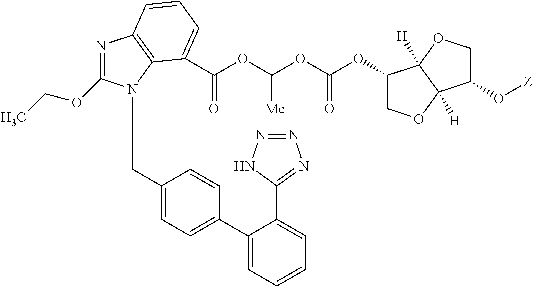

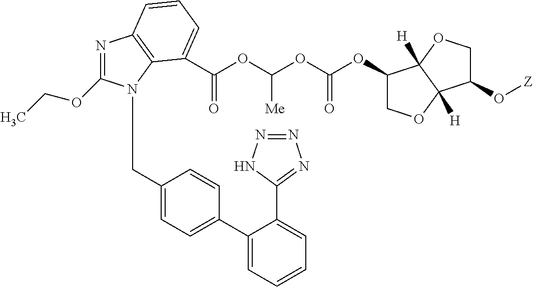

| Z | Z | Z |
|---|---|---|
| 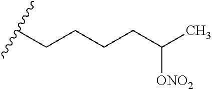 | 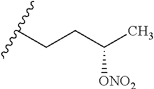 | 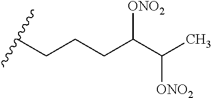 |
| 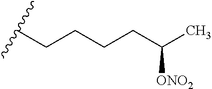 | 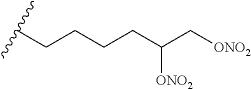 | 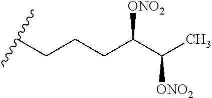 |
| 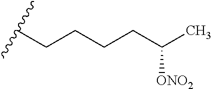 | 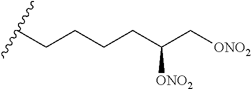 | 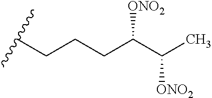 |
| 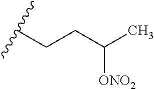 | 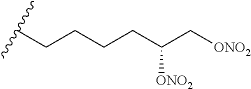 | 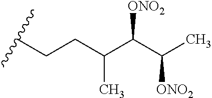 |
| 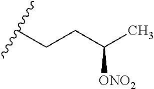 | 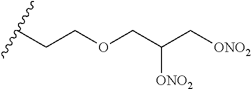 | 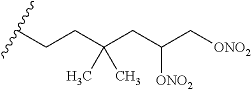 |
| 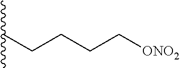 | 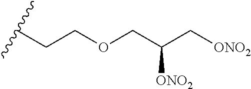 | 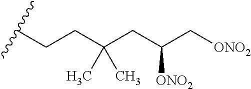 |
| 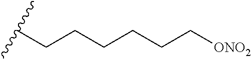 | 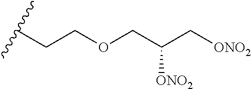 | 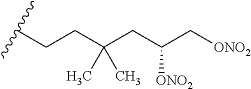 |

TABLE 6

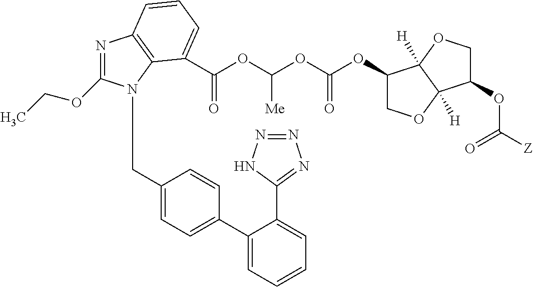

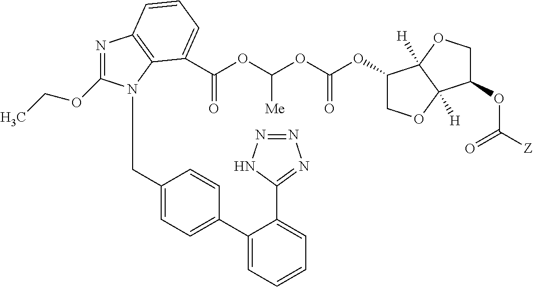

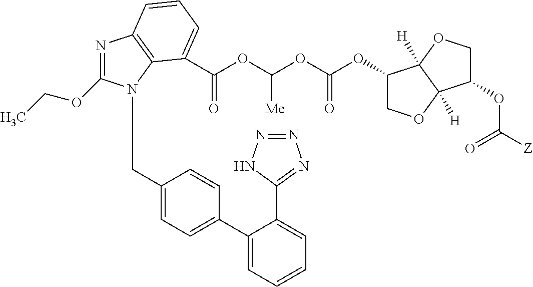

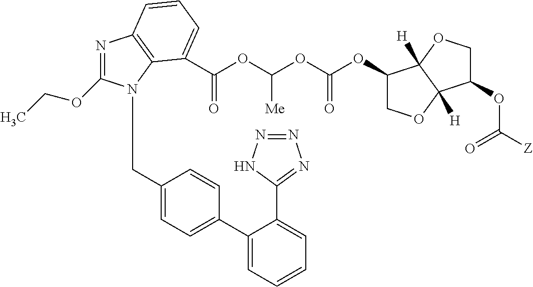

| Z | Z | Z |
|---|---|---|
| 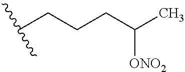 | 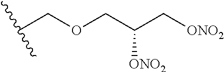 | 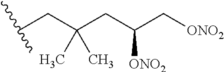 |
| 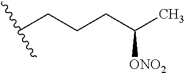 | 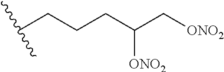 | 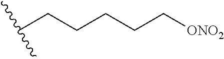 |
| 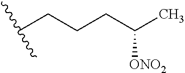 | 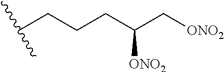 | 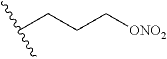 |
| 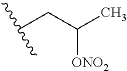 | 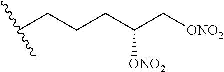 | 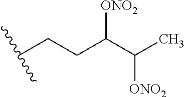 |
| 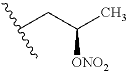 | 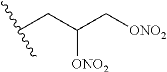 | 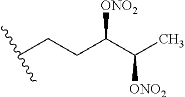 |
| 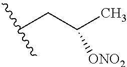 | 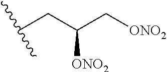 | 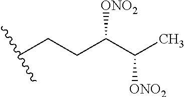 |
| 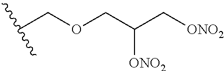 | 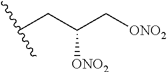 | 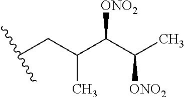 |
| 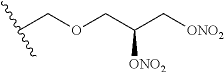 | 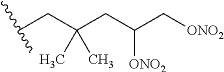 | 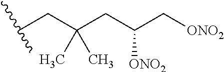 |

TABLE 7

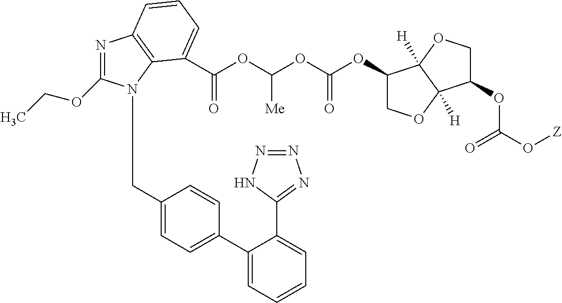

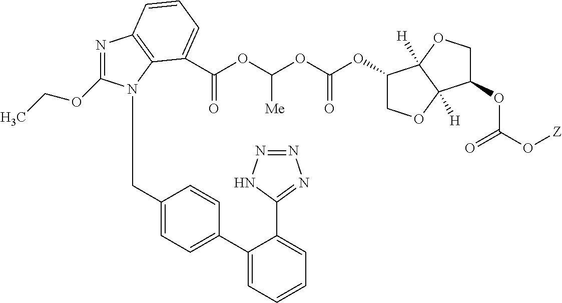

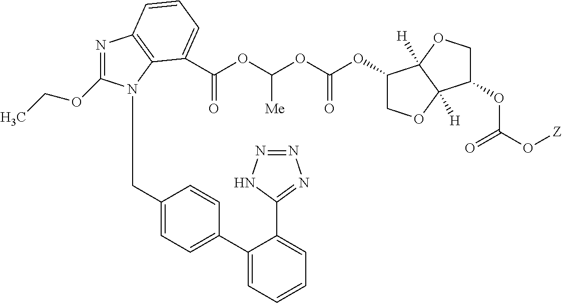

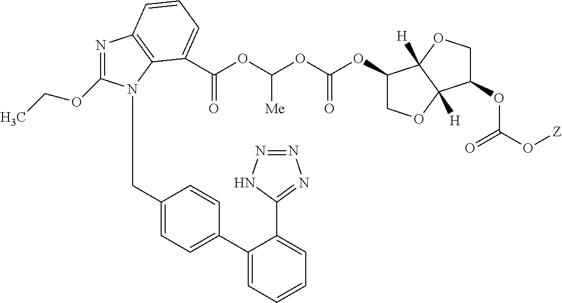

| Z | Z | Z |
|---|---|---|
| 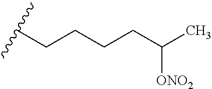 | 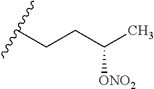 | 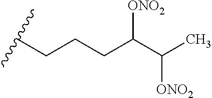 |
| 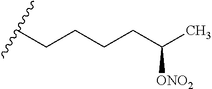 | 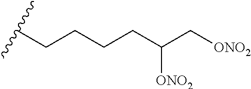 | 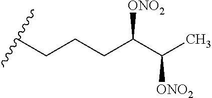 |
| 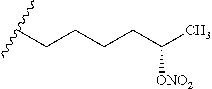 | 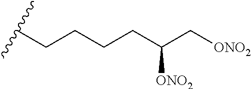 | 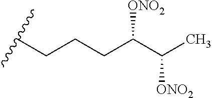 |
| 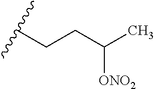 | 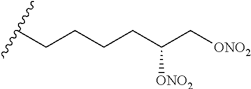 | 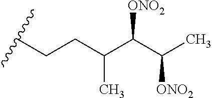 |
| 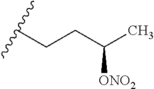 | 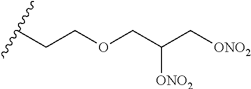 | 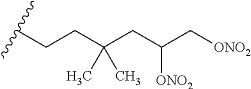 |
| 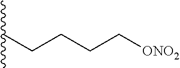 | 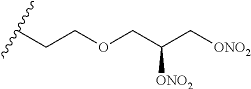 | 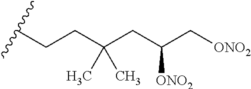 |
| 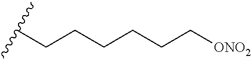 | 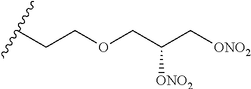 | 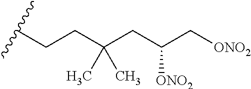 |

TABLE 8

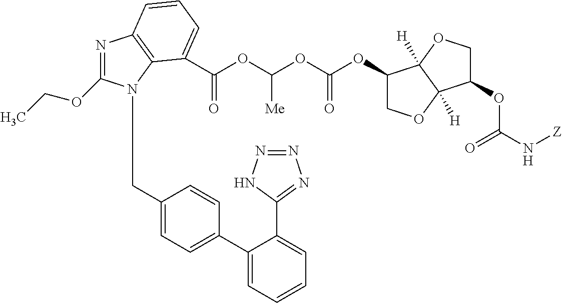

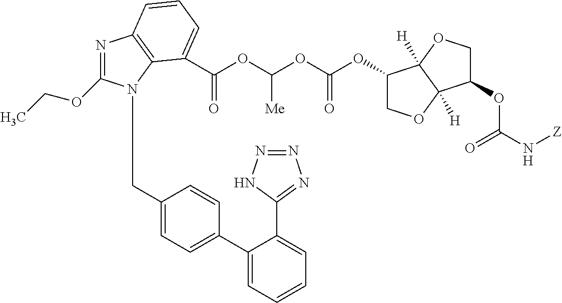

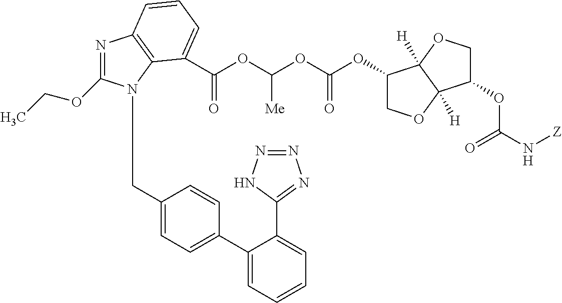

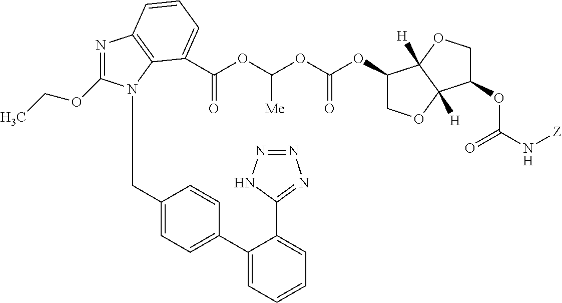

| Z | Z | Z |
|---|---|---|
| 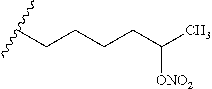 | 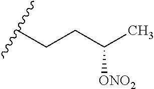 | 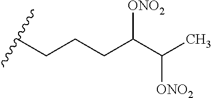 |
| 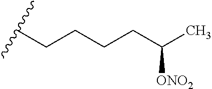 | 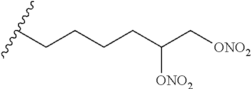 | 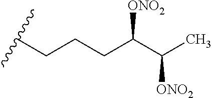 |
| 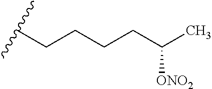 | 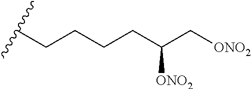 | 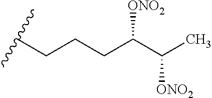 |
| 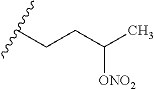 | 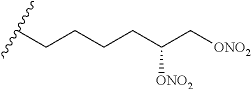 | 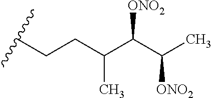 |
| 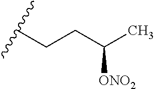 | 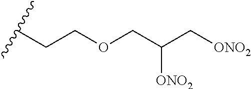 | 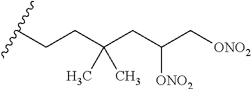 |
| 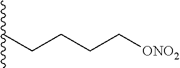 | 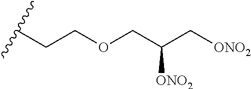 | 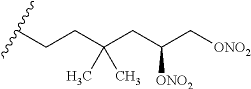 |
| 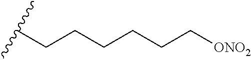 | 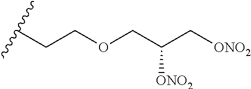 | 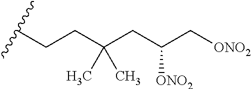 |

TABLE 9

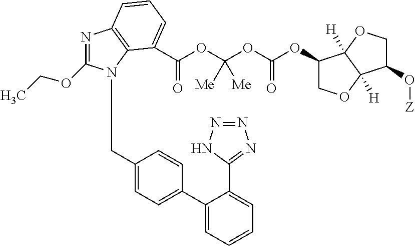

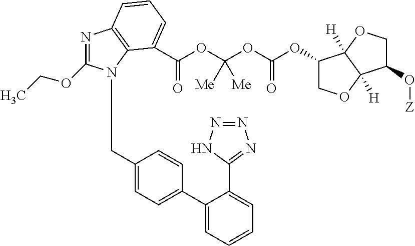

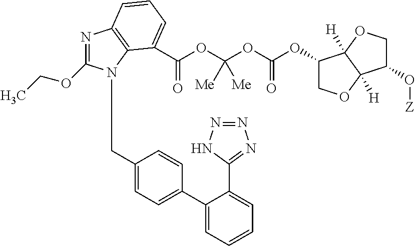

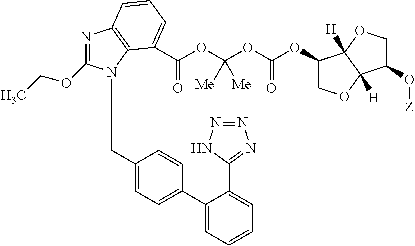

| Z | Z |
|---|---|
| 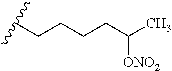 | 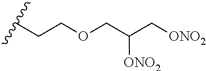 |
| 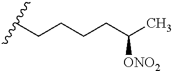 | 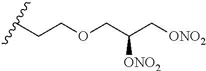 |
| 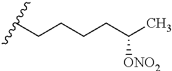 | 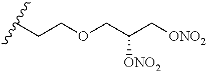 |
| 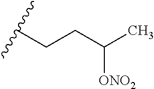 | 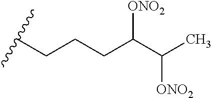 |
| 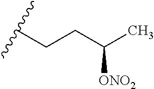 | 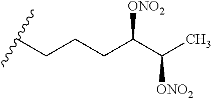 |
| 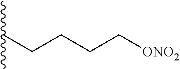 | 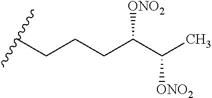 |
| 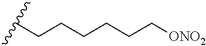 | 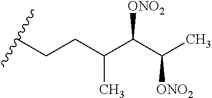 |
| 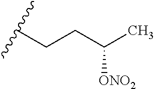 | 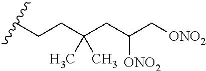 |
| 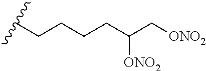 | 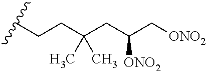 |
| 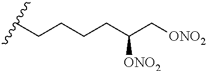 | 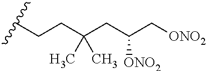 |
| 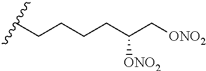 | |

TABLE 10

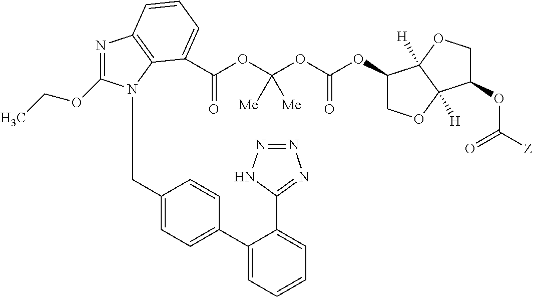

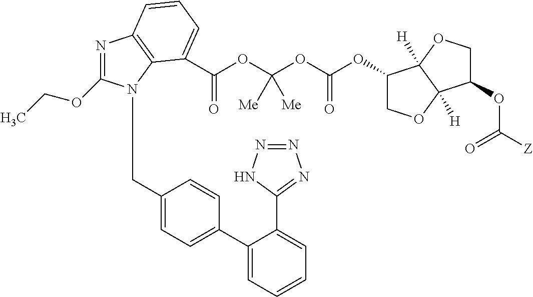

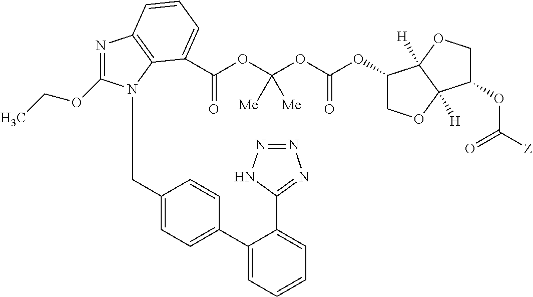

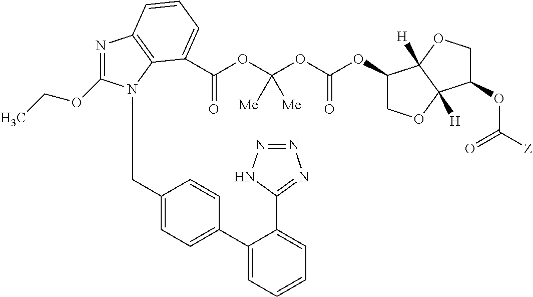

| Z | Z | Z |
|---|---|---|
| 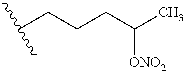 | 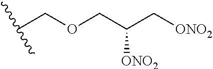 | 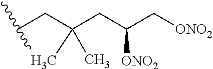 |
| 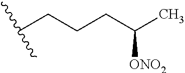 | 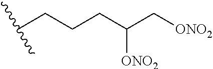 | 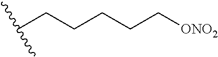 |
| 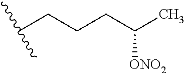 | 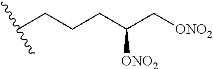 | 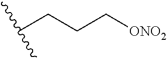 |
| 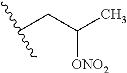 | 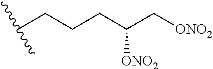 | 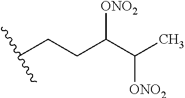 |
| 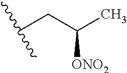 | 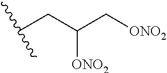 | 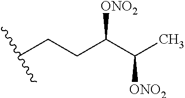 |
| 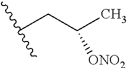 | 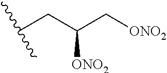 | 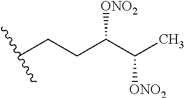 |
| 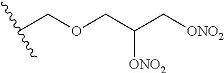 | 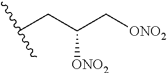 | 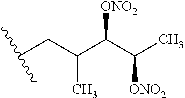 |
| 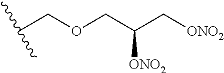 | 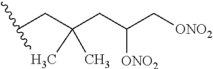 | 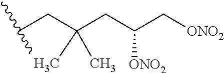 |

TABLE 11

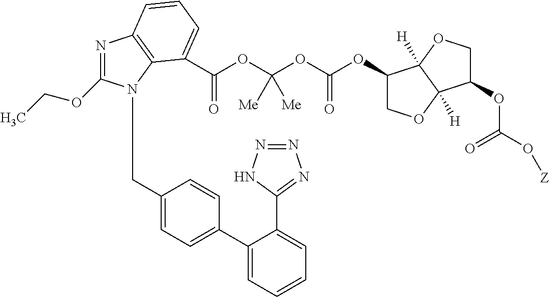

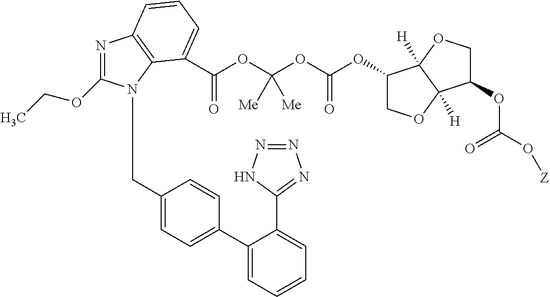

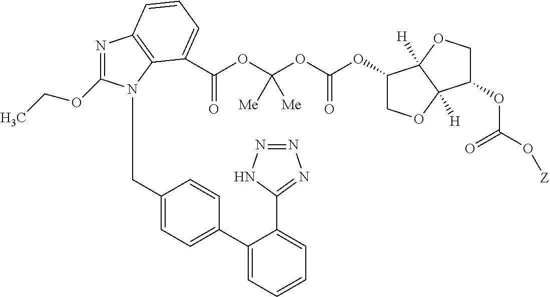

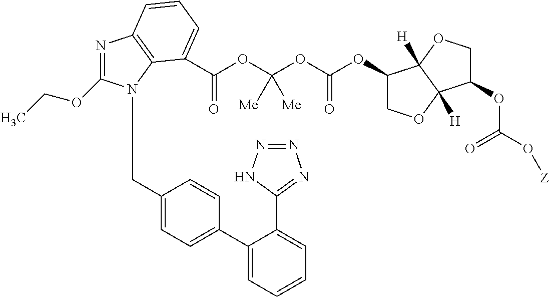

| Z | Z | Z |
|---|---|---|
| 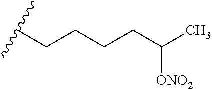 | 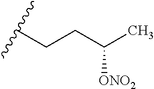 | 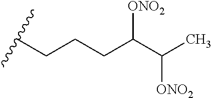 |
| 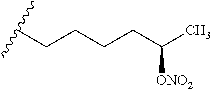 | 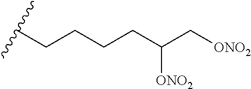 | 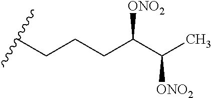 |
| 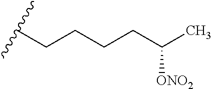 | 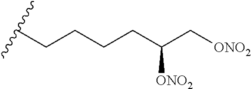 | 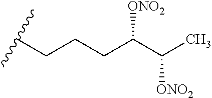 |
| 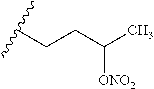 | 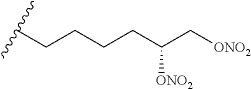 | 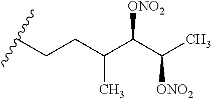 |
| 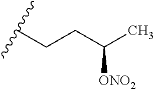 | 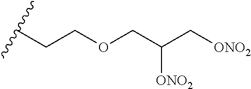 | 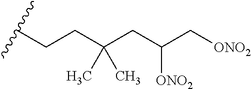 |
| 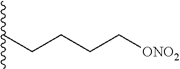 | 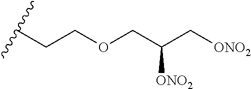 | 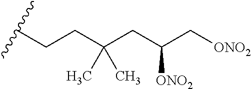 |
| 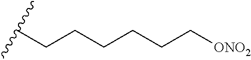 | 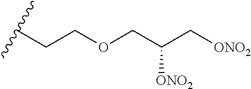 | 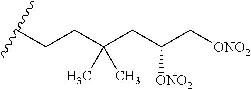 |

TABLE 12

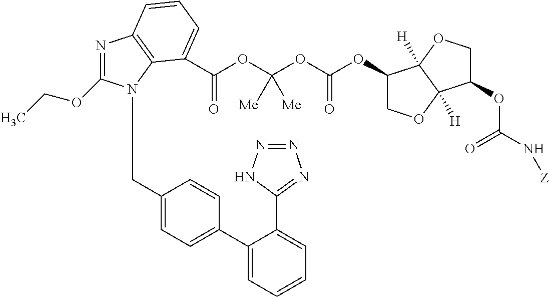

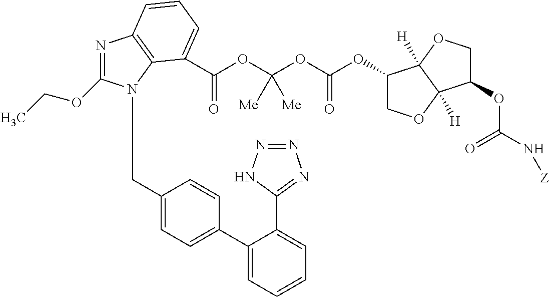

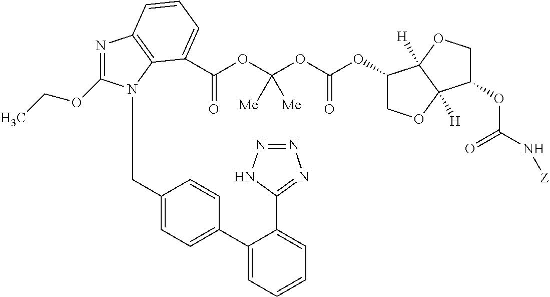

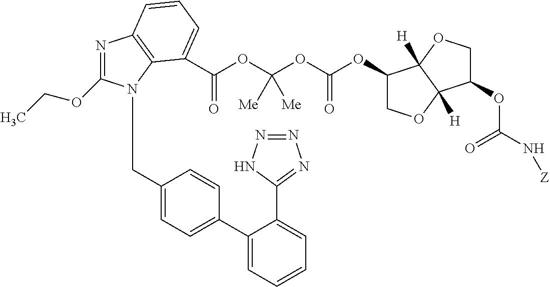

| Z | Z | Z |
|---|---|---|
| 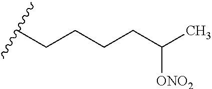 | 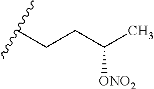 | 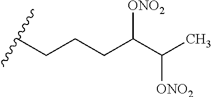 |
| 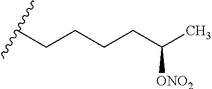 | 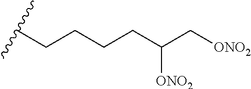 | 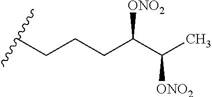 |
| 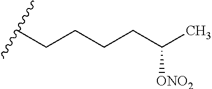 | 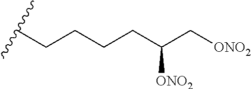 | 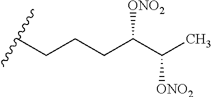 |
| 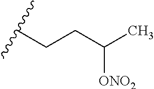 | 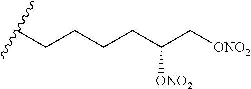 | 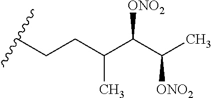 |
| 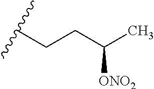 | 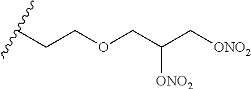 | 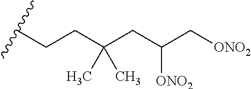 |
| 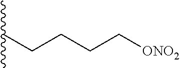 | 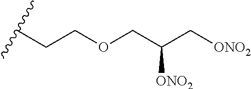 | 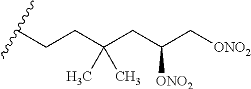 |
| 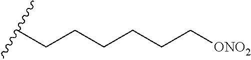 | 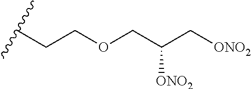 | 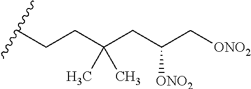 |

TABLE 13

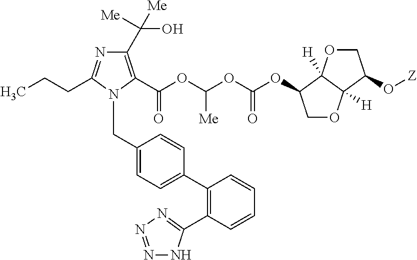

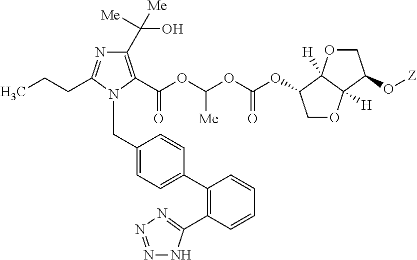

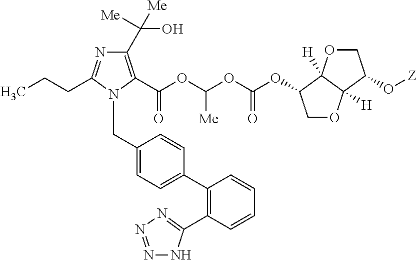

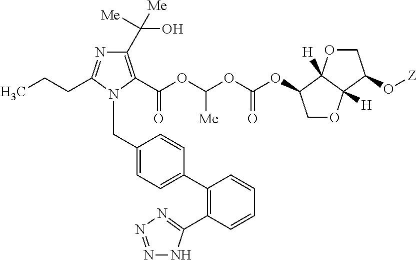

| Z | Z |
|---|---|
| 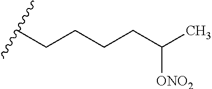 | 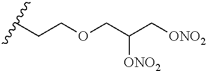 |
| 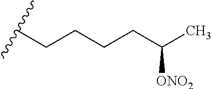 | 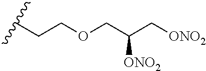 |
| 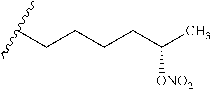 | 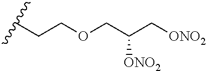 |
| 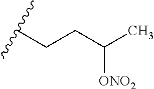 | 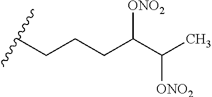 |
| 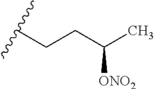 | 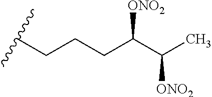 |
| 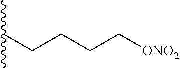 | 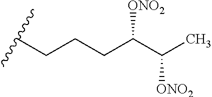 |
| 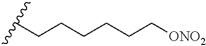 | 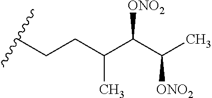 |
| 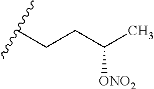 | 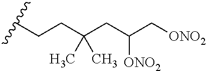 |
| 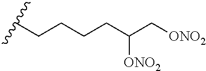 | 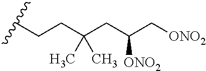 |
| 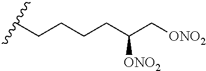 | 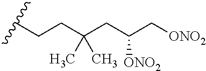 |
| 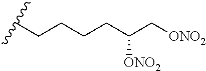 | |

TABLE 14

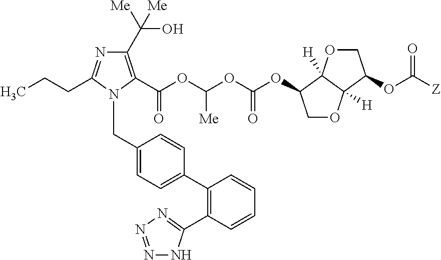

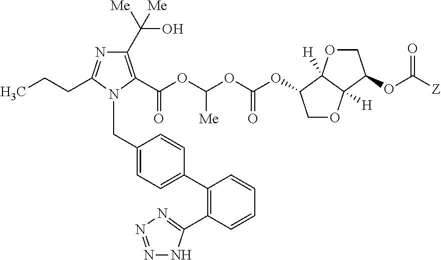

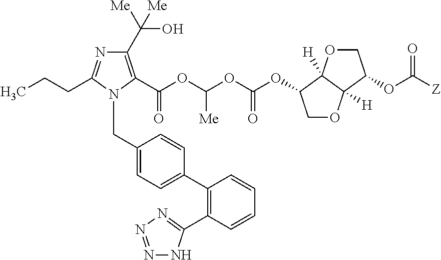

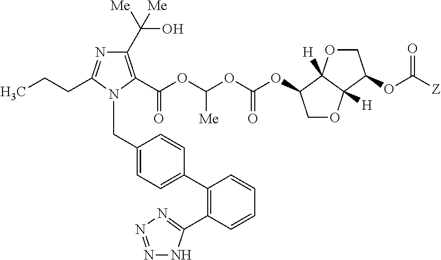

| Z | Z |
|---|---|
| 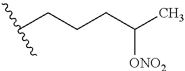 | 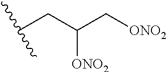 |
| 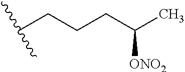 | 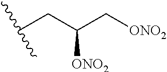 |
| 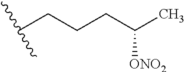 | 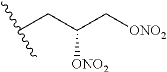 |
| 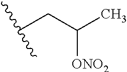 | 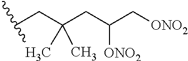 |
| 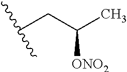 | 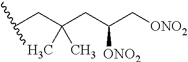 |
| 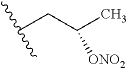 | 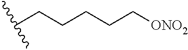 |
| 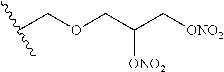 | 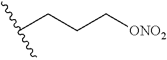 |
| 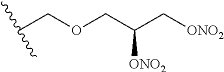 | 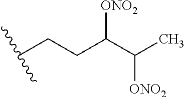 |
| 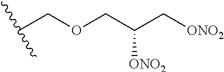 | 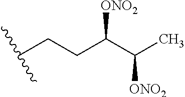 |
| 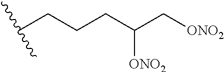 | 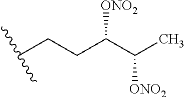 |
| 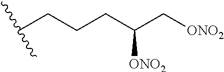 | 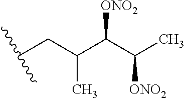 |
| 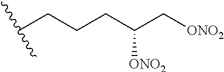 | 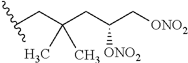 |

TABLE 15

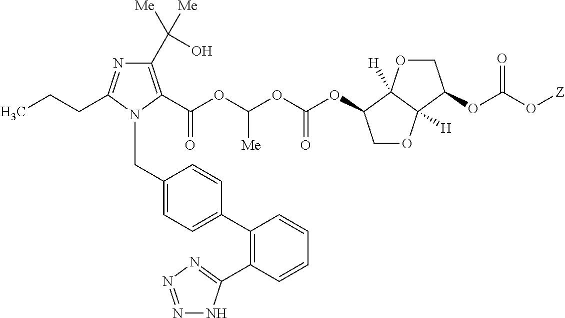

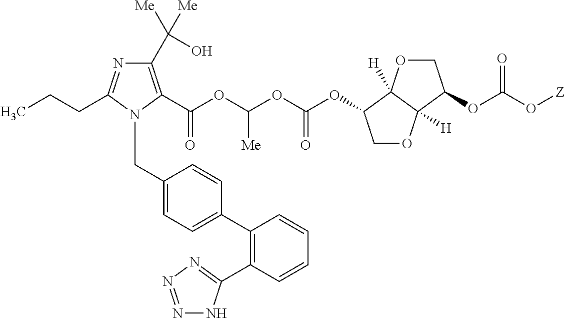

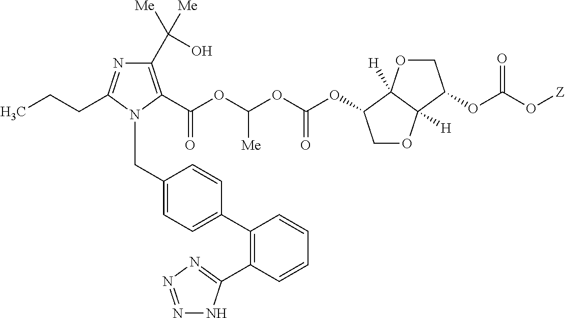

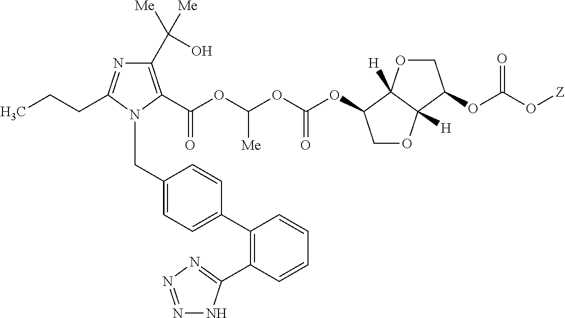

| Z | Z | Z |
|---|---|---|
| 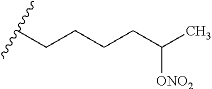 | 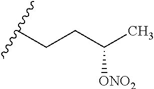 | 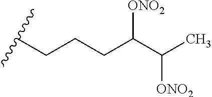 |
| 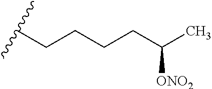 | 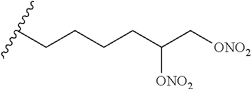 | 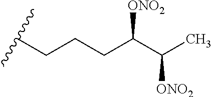 |
| 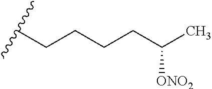 | 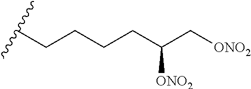 | 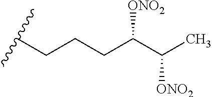 |
| 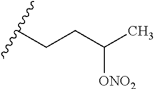 | 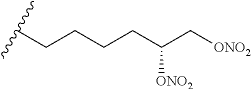 | 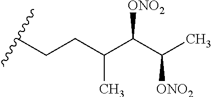 |
| 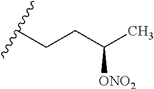 | 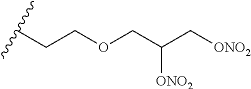 | 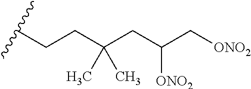 |
| 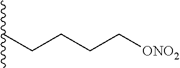 | 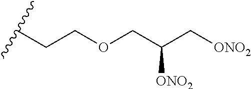 | 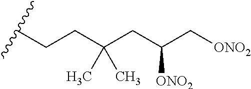 |
| 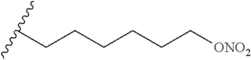 | 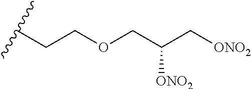 | 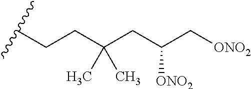 |

TABLE 16

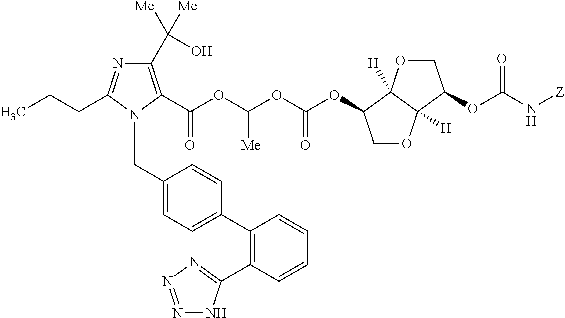

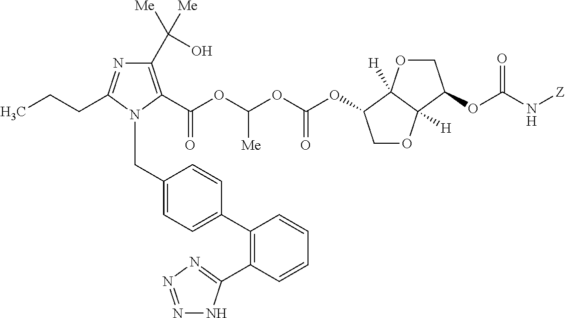

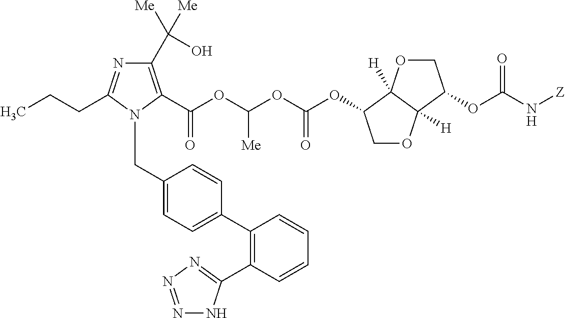

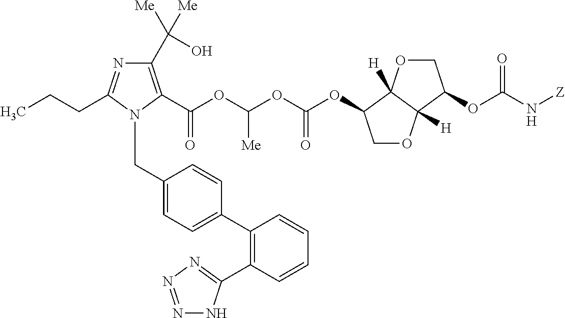

| Z | Z | Z |
|---|---|---|
| 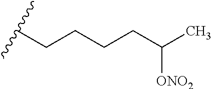 | 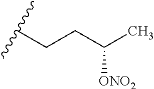 | 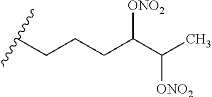 |
| 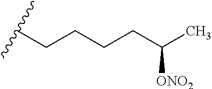 | 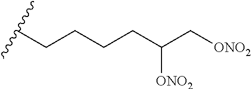 | 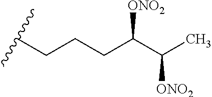 |
| 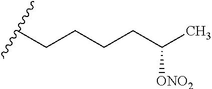 | 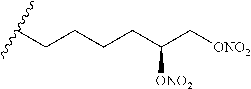 | 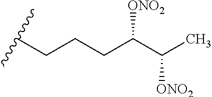 |
| 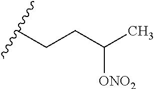 | 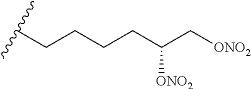 | 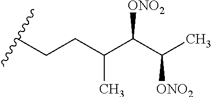 |
| 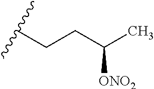 | 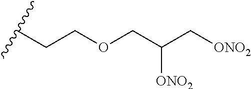 | 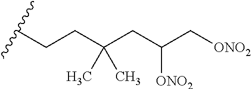 |
| 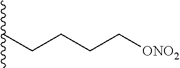 | 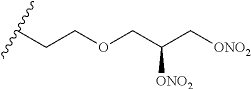 | 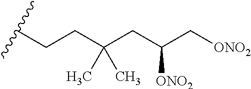 |
| 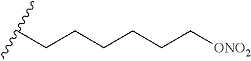 | 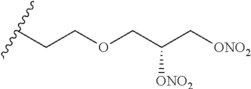 | 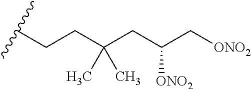 |

TABLE 17

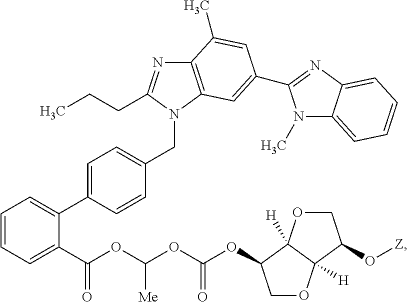

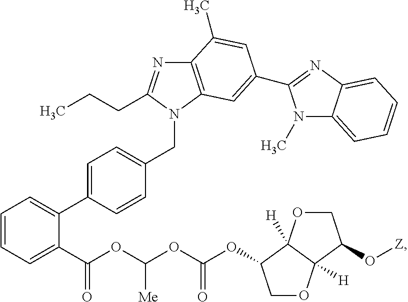

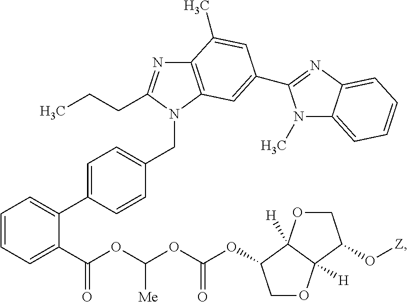

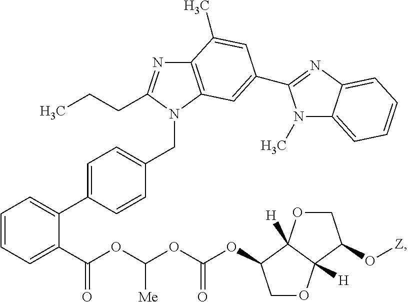

Z

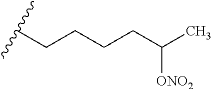

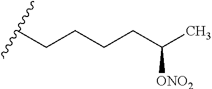

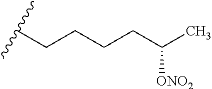

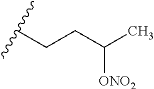

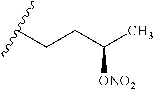

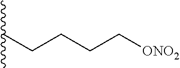

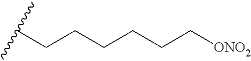

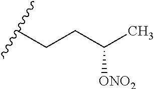

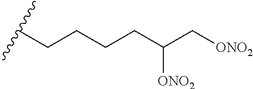

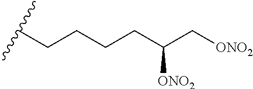

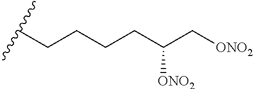

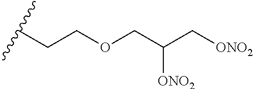

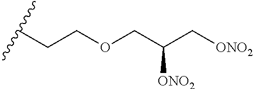

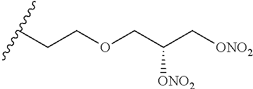

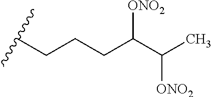

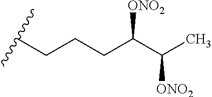

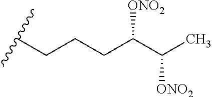

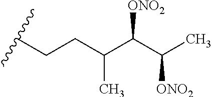

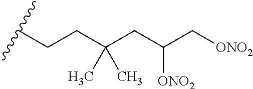

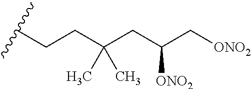

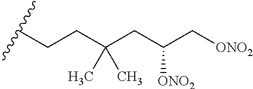

TABLE 18

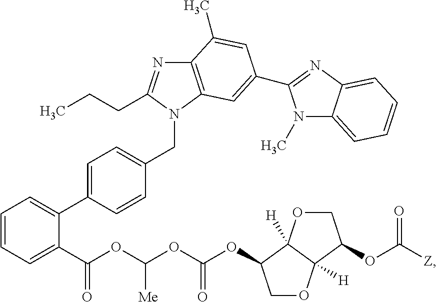

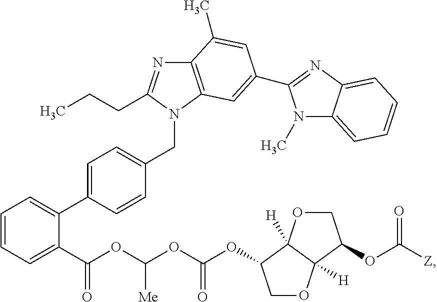

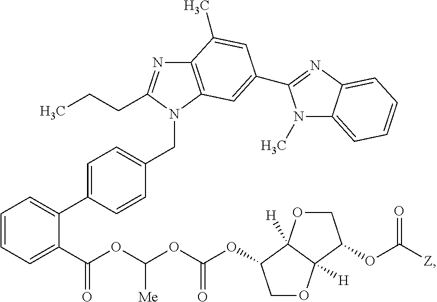

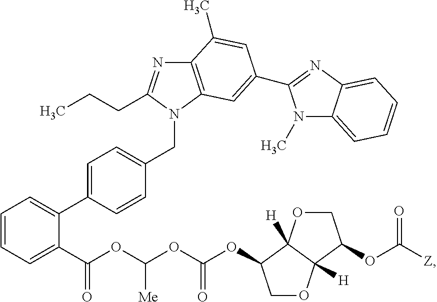

Z

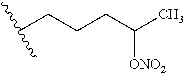

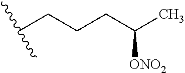

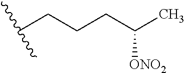

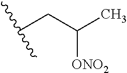

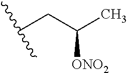

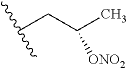

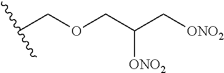

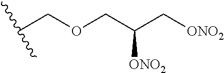

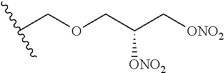

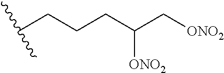

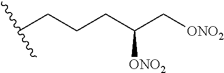

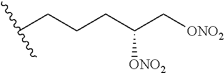

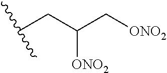

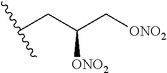

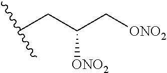

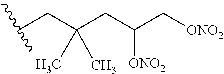

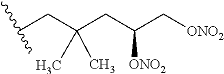

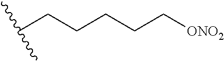

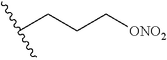

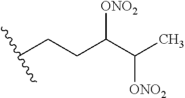

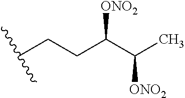

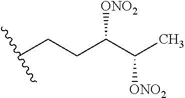

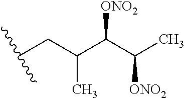

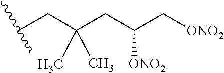

TABLE 19

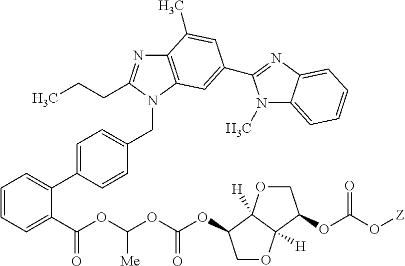

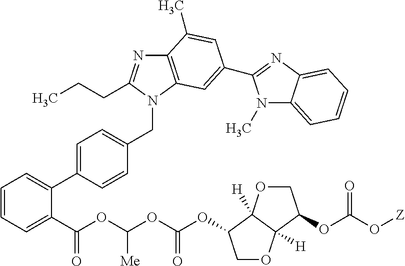

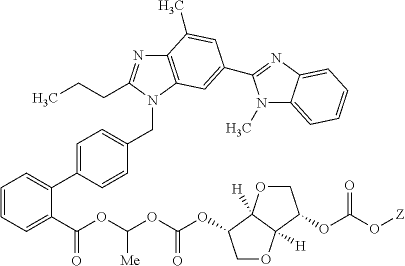

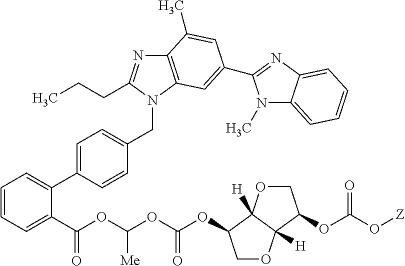

Z

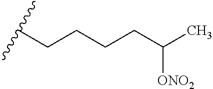

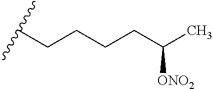

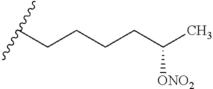

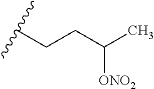

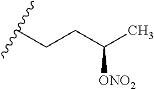

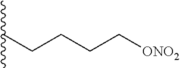

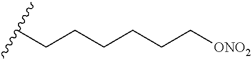

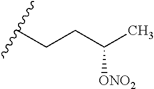

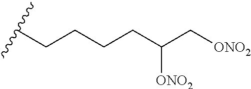

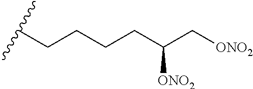

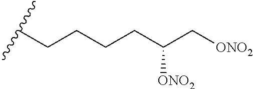

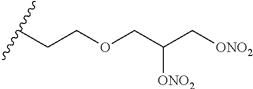

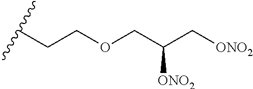

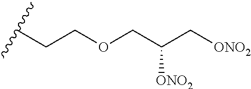

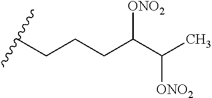

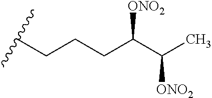

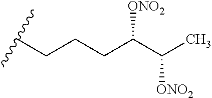

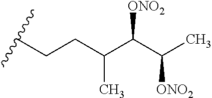

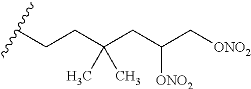

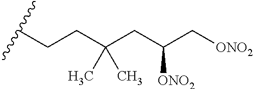

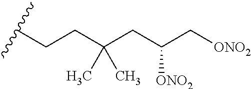

TABLE 20

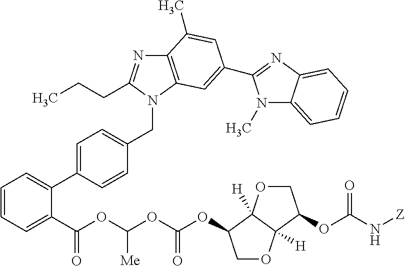

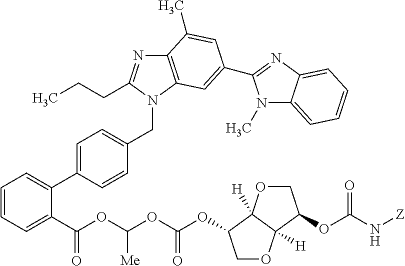

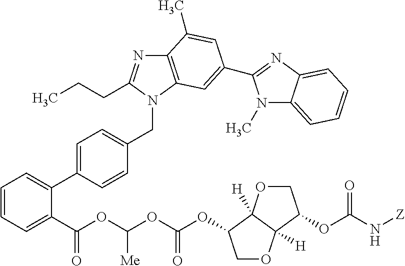

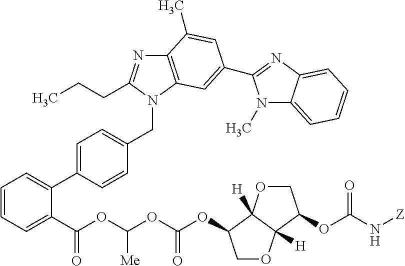

Z

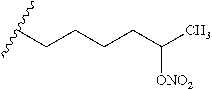

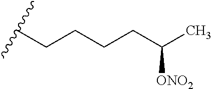

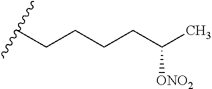

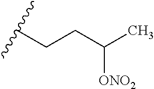

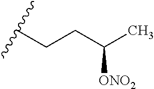

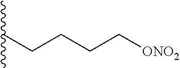

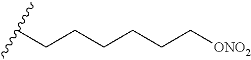

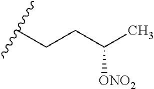

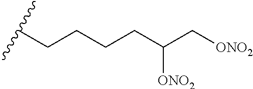

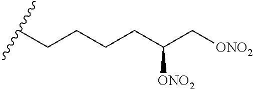

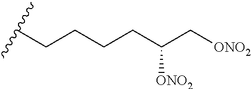

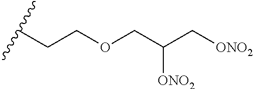

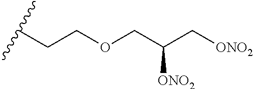

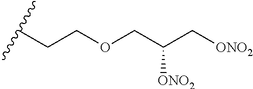

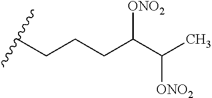

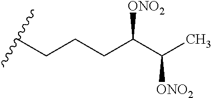

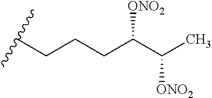

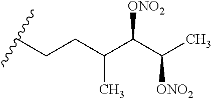

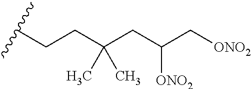

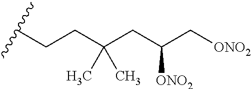

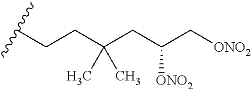

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the object of the invention are also all the possible isomers, stereoisomers and their mixtures of the compounds of formula (I).

As used herein except where noted, “alkyl” is intended to include both branched- and straight-chain saturated aliphatic hydrocarbon groups having the specified number of carbon atoms. Commonly used abbreviations for alkyl groups are used throughout the specification, e.g. methyl may be represented by conventional abbreviations including “Me” or CH₃ or a symbol that is an extended bond as the terminal group, e.g.

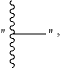

ethyl may be represented by “Et” or CH₂CH₃, propyl may be represented by “Pr” or CH₂CH₂CH₃, butyl may be represented by “Bu” or CH₂CH₂CH₂CH₃, etc. “C₁₋₄ alkyl” (or “C₁-C₄ alkyl”) for example, means linear or branched chain alkyl groups, including all isomers, having the specified number of carbon atoms. C₁₋₄ alkyl includes n-, iso-, sec- and t-butyl, n- and isopropyl, ethyl and methyl. If no number is specified, 1-4 carbon atoms are intended for linear or branched alkyl groups.

The term “C₁-C₁₀ alkylene” as used herein refers to branched or straight chain C₁-C₁₀ hydrocarbon such as methylene, ethylene, propylene, isopropylene, n-butylene, pentylene, n-hexylene and the like. The angiotensin II receptor antagonists (ARBs) of the invention are useful for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system.

The ARBs of the invention are especially useful for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In one embodiment, the invention relates to a method for the treatment and/or prophylaxis of diseases, which are associated with a dysregulation of the renin-angiotensin system, in particular to a method for the treatment or prophylaxis of the above-mentioned diseases, said methods comprising administering to a patient a pharmaceutically active amount of an angiotensin II receptor antagonist of the invention.

The invention also relates to the use of ARBs of the invention for the preparation of a medicament for the treatment and/or prophylaxis of the above-mentioned diseases.

The above-mentioned ARBs of the invention are also of use in combination with other pharmacologically active compounds comprising angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril; ramipril, spirapril, temocapril, or trandolapril), neutral endopeptidase inhibitors (e.g., thiorphan and phosphoramidon), aldosterone antagonists, renin inhibitors (e.g. urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives (U.S. Pat. No. 5,106,835), peptidyl amino diols (U.S. Pat. Nos. 5,063,208 and 4,845,079) and peptidyl beta-aminoacyl aminodiol carbamates (U.S. Pat. No. 5,089,471); also, a variety of other peptide analogs as disclosed in the following U.S. Pat. Nos. 5,071,837; 5,064,965; 5,063,207; 5,036,054; 5,036,053; 5,034,512 and 4,894,437, and small molecule renin inhibitors (including diol sulfonamides and sulfinyls (U.S. Pat. No. 5,098,924), N-morpholino derivatives (U.S. Pat. No. 5,055,466), N-heterocyclic alcohols (U.S. Pat. No. 4,885,292) and pyrolimidazolones (U.S. Pat. No. 5,075,451); also, pepstatin derivatives (U.S. Pat. No. 4,980,283) and fluoro- and chloro-derivatives of statone-containing peptides (U.S. Pat. No. 5,066,643), enalkrein, RO 42-5892, A 65317, CP 80794, ES 1005, ES 8891, SQ 34017, aliskiren ((2S,4S,5S,7S)—N-(2-carbamoyl-2-methylpropyl)-5-amino-4-hydroxy-2,7-diisopropyl-8-[4-methoxy-3-methoxypropoxy)phenyl]-octanamidhemifumarate) SPP600, SPP630 and SPP635), endothelin receptors antagonists, vasodilators, calcium channel blockers (e.g., amlodipine, nifedipine, veraparmil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazocin, prazosin or alpha methyldopa) central alpha adrenergic agonists, peripheral vasodilators (e.g. hydralazine), lipid lowering agents (e.g., simvastatin, lovastatin, ezetimibe, atorvastatin, pravastatin), metabolic altering agents including insulin sensitizing agents and related compounds (e.g., muraglitazar, glipizide, metformin, rosiglitazone)) or with other drugs beneficial for the prevention or the treatment of the above-mentioned diseases including nitroprusside and diazoxide.

The dosage regimen utilizing the angiotensin II receptor antagonists is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the angiotensin II receptor antagonists, when used for the indicated effects, will range between about 0.0125 mg per kg of body weight per day (mg/kg/day) to about 7.5 mg/kg/day, preferably 0.0125 mg/kg/day to 3.75 mg/kg/day, and more preferably 0.3125 mg/kg/day to 1.875 mg/kg/day. For example, an 80 kg patient would receive between about 1 mg/day and 600 mg/day, preferably 1 mg/day to 300 mg/day, and more preferably 25 mg/day to 150 mg/day. A suitably prepared medicament for once a day administration would thus contain between 1 mg and 600 mg, preferably between 1 mg and 300 mg, and more preferably between 25 mg and 300 mg, e.g., 25 mg, 50 mg, 100 mg, 150, 200, 250 and 300 mg. Advantageously, the angiotensin II receptor antagonists may be administered in divided doses of two, three, or four times daily. For administration twice a day, a suitably prepared medicament would contain between 0.5 mg and 300 mg, preferably between 0.5 mg and 150 mg, more preferably between 12.5 mg and 150 mg, e.g., 12.5 mg, 25 mg, 50 mg, 75 mg, 100 mg, 125 mg and 150 mg.

The angiotensin II receptor antagonists of the invention can be administered in such oral forms as tablets, capsules and granules. The angiotensin II receptor antagonists are typically administered as active ingredients in admixture with suitable pharmaceutical binders as described below. % w/w expresses the weight percent of the indicated composition constituent compared to the total composition. Suitable fillers used in these dosage forms include microcrystalline cellulose, silicified microcrystalline cellulose, dicalcium phosphate, lactose, mannitol, and starch, preferably microcrystalline cellulose, dicalcium phosphate, lactose or mixtures thereof. Suitable binders include hydroxypropyl cellulose, hydroxypropyl methyl cellulose, starch, gelatin, natural sugars such as glucose or beta-lactose, corn-sweeteners, natural and synthetic gums such as acacia, tragacanth or sodium alginate, carboxymethylcellulose, and polyvinyl pyrrolidone. Lubricants used in these dosage forms include sodium oleate, sodium stearate, magnesium stearate, sodium benzoate, sodium acetate, sodium chloride, sodium stearyl fumarate, stearic acid and the like, preferably magnesium stearate. Suitable coating compositions include aqueous dispersion or organic solution of insoluble polymers such as ethyl cellulose, cellulose aetate, cellulose acetate butyrate and acrylate copolymers commercially known as Eudragit®. Plasticizers include triethyl citrate, dibutyl sebacate, dibutyl phthalate, triacetin and castor oil. Antitacking agents include talc, kaolin, colloidal silica or mixtures thereof.

### General Synthesis

1. The compounds of general formula (I) as above defined

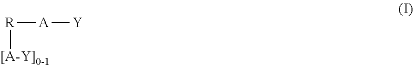

wherein
R is selected from the group consisting of (IIa)-(IIh) as above defined;
Y is as above defined and is equal to X⁰—Z

### A is

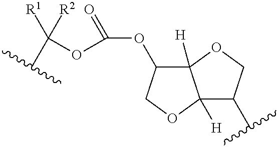

wherein R¹ and R² are as above defined,
can be obtained by a process comprising reacting a compound of formula (IIIa)-(IIIh):

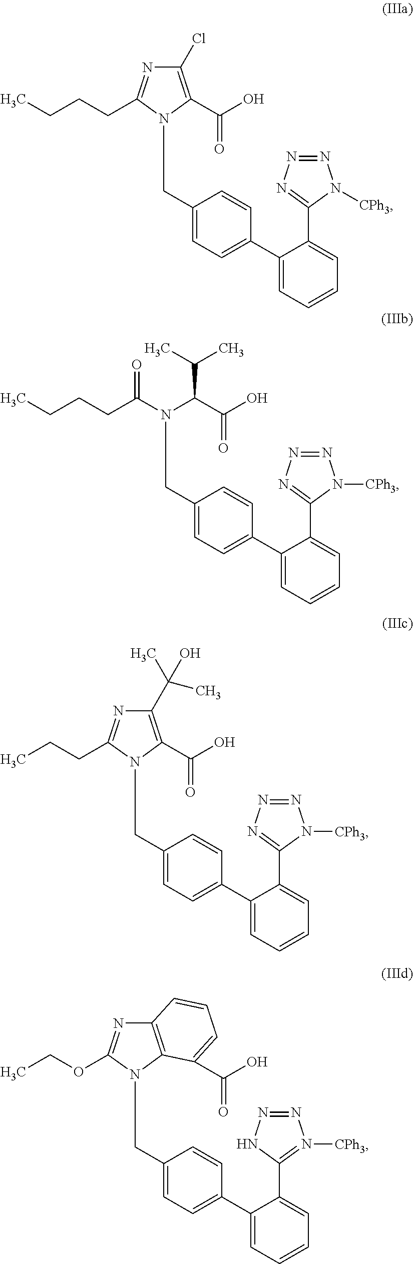

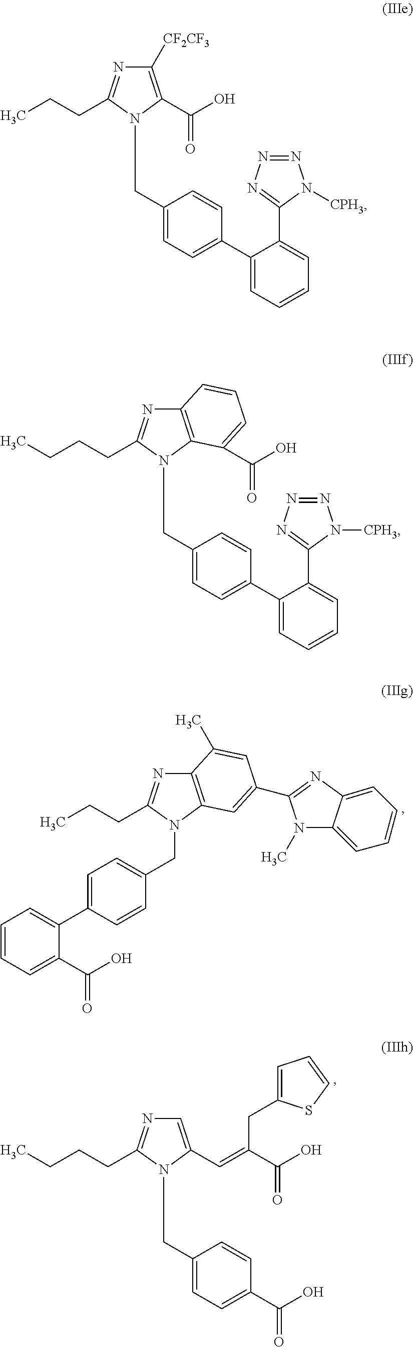

with a compound of formula (IVa):

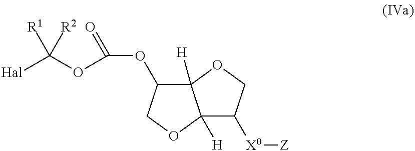

wherein Hal is an halogen atom such as Cl, Br, I; R¹, R² and X⁰—Z are as above defined, in the presence of an organic or inorganic base such as TEA, pyridine or CsCO₃, with or without a catalytic amount of NaBr or NaI, in an aprotic polar/non polar solvent such as THF, DMF or CH₂Cl₂, at temperatures ranging between 20° C. to 160° C., eventually removing the trityl group when present following methods well known in the art.

Compounds of formula (IVa) can be prepared reacting a compound of formula (IVb), with commercial or known in the art HalCR¹R²OCOCl in the presence of an organic or inorganic base such as in an aprotic polar/non polar solvent such as THF, DMF or CH₂Cl₂, at temperatures ranging between 0° C. to 100° C.

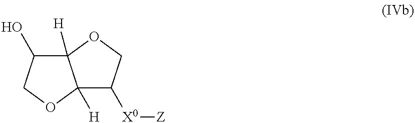

Compounds of formula (IVb) wherein X⁰ is —O— and Z is as above defined can be prepared by reacting compounds (IVc) or (IVd):

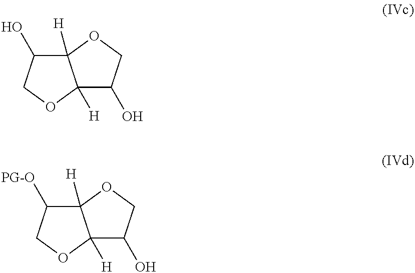

wherein PG is a protective group such as allyl, trialkylsilyl, tetrahydropiranyl with a compound of formula (Va)

Hal-Z (Va)

wherein Hal is an Halogen atom such as —Cl, —Br, —I;
in the presence of an organic or inorganic base such as NaH, DBU, in an aprotic polar solvent such as THF, DMF at temperatures ranging between −20° C. to 100° C., eventually removing the protective group, when present, with methods known in the literature.

Alternatively compound (IVb) or (IVc) can be reacted with a compound of formula (Vaa) Hal-Z′ wherein Z′ contains precursors of the group —ONO₂ such as double bond, and/or the group —OH or its precursor as for example a carbonyl group, affording compounds (VIa):

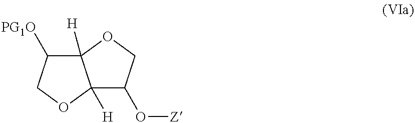

wherein PG¹ is H or a protective group. Compound (VIa) can be converted into compound (IVb) by nitrating procedure well known in the art, eventually removing the protective group when present.

Compounds (Va) or compounds (Vaa) are known in the literature or can be prepared by known compounds by known procedures.
Compounds of formula (IVb) wherein X⁰ is —OCO— and Z is as above defined can be prepared by reacting compounds (IVc) or (IVd) with a compound of formula (Vb)

WOC—Z (Vb)

wherein W is —OH or a carboxylic activating group such as C₆F₅—O and 4-NO₂—C₆H₄—O— or is Hal wherein Hal is an Halogen atom such as —Cl, —F; Z is as above defined:
depending on the meaning of W, in the presence of a condensing agent such as DCC or CDI, or EDC, or other well known in the art, or in the presence of an organic or inorganic base such as TEA, DIPEA, DBU, in an aprotic polar/apolar solvent such as THF, DMF CH₂Cl₂ at temperatures ranging between −20° C. to 100° C., following esterification methods well known in the literature. Eventually removing the protective group, when present, with methods known in the literature.

Alternatively compounds of formula (IVb) wherein X⁰ is —OCO— and Z is as above defined can be prepared by reacting compounds (IVc) or (IVd) with a compound of formula (Vbb) WOC—Z′ wherein W and Z′ are as above defined and Z′ contain precursors of the group —ONO₂ such as double bond, or an halogen atom such as Cl, B, I, affording compounds (VIb):

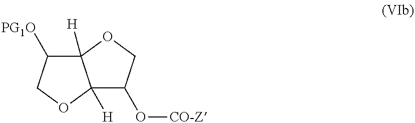

wherein PG₁ and Z′ are as above defined. Compound (VIb) can be converted into compound (Vb) by nitrating procedure well known in the art, eventually removing the protective group. Compounds (Vb) and (Vbb) are known in the art or can be prepared by known compound following known procedure.
Compounds of formula (IVb) wherein X⁰ is —OCOO— and Z is as above defined can be prepared by reacting compounds (IVe):

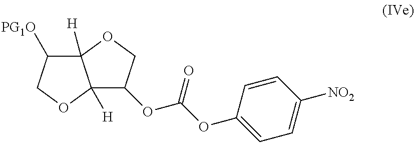

wherein PG₁ is as above defined, with compounds HO—Z (Vc) or compound HO—Z′ (Vcc) wherein Z and Z′ are as above defined, in the presence of an organic or inorganic base such as DMAP, TEA, DIPEA, with or without catalyst such as Sc(OTf)₃ in an aprotic polar/apolar solvent such as THF, DMF CH₂Cl₂ at temperatures ranging between −20° C. to 100° C., following methods well known in the literature. Eventually nitrating the precursor if Z′ is present and removing the protective group, with methods known in the literature.
Compounds of formula (IVb) wherein X⁰ is —OCONH— and Z is as above defined can be prepared by reacting compounds (IVe) with compounds (Vd) Z—NH₂ or (Vdd) Z′—NH₂ in the presence of an organic or inorganic base such as DMAP, TEA, DIPEA, with or without catalyst such as Sc(OTf)₃ in an aprotic polar/apolar solvent such as THF, DMF CH₂Cl₂ at temperatures ranging between −20° C. to 100° C., following methods well known in the literature. Eventually nitrating the precursor if Z′ is present and removing the protective group, with methods known in the literature.

2. The compound of general formula (I)

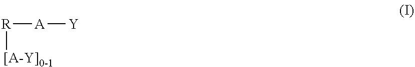

wherein R is (IId) as above defined, Y is as above defined,

### and A is:

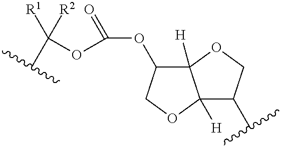

wherein R¹ and R² are CH₃, can be obtained by a process comprising reacting a compound of formula (IIIdd):

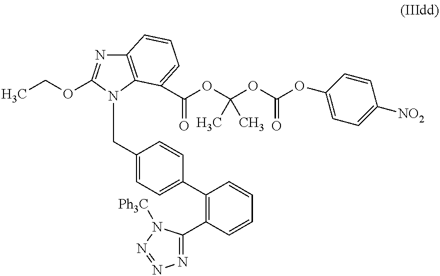

with a compound of formula (IVb) as above defined, in the presence of a stoichiometric amount of DMAP and with or without a catalytic amount of a triflate salt like Sc(OTf)₃ in solvent such as DMF or CH₂Cl₂, at temperatures ranging between 20° C. to 100° C., eventually removing the trityl group following methods well known in the art.

Compounds of formula (IIIdd) can be prepared by reacting compounds of formula (IIId) as above defined with a compound (VIIa), known in the literature (Alexander, J. U.S. Pat. No. 5,684,018):

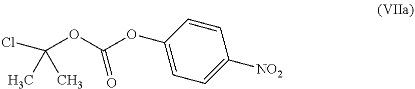

in the presence of an organic or inorganic base such as TEA, pyridine or CsCO₃, with or without a catalytic amount of NaBr or NaI, in an aprotic polar/non polar solvent such as THF, DMF or CH₂Cl₂, at temperatures ranging between 20° C. to 100° C.

The following examples are to further illustrate the invention without limiting it:

### Example 1

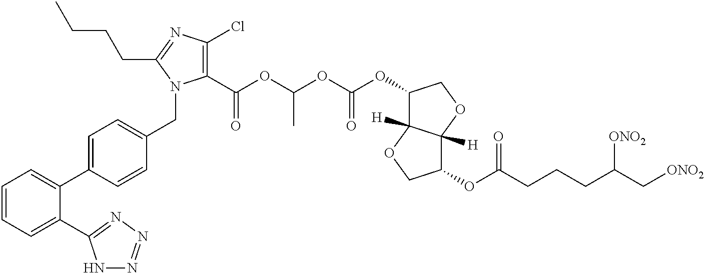

### 1-(((3R,3aR,6R,6aR)-6-(5,6-bis(nitrooxy)hexanoyloxy)hexahydrofuro[3,2-b]furan-3-yloxy)carbonyloxy)ethyl 1-((2′-(1H-tetrazol-5-yl)biphenyl-4-yl)methyl)-2-butyl-4-chloro-1H-imidazole-5-carboxylate

### Step A: (2-butyl-4-chloro-1-{[2′-(1H-tetrazol-5-yl)biphenyl-4-yl]methyl}-1H-imidazole-5-carboxylic acid (E3174)

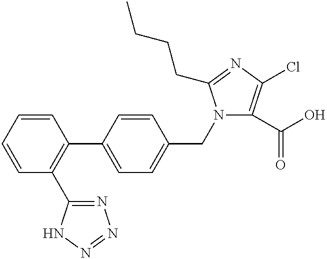

Water (10 L) was added to a 22 L 4-neck round bottom flask. The water was cooled to 0° C. At 0° C., potassium hydroxide (855 g, 15.24 mol) was added followed by losartan potassium (500 g, 1.09 mol)), sodium periodate (554 g, 2.59 mol) and ruthenium (III) chloride hydrate (12 g, 0.05 mol) and the reaction mixture was stirred at 0° C. overnight. The reaction mixture was filtered. IPA (90 mL) was added to the filtrate while stirring. The solution was warmed to 25° C. and stirred for 2.5 hrs. After 2.5 hrs., phosphoric acid (1200 mL) was added, maintaining the temperature below +30° C. The mixture was stirred for 30 min and the product was filtered, washing with water. The residue was dried in the vacuum oven at 55° C. overnight. The solid was dissolved in methanol (4 L) and isopropyl acetate (12 L), and charcoal (activated carbon) (100 g) was added. The mixture was stirred at rt for 3.5 hrs, filtered and concentrated. The product was redissolved in DCM/MeOH and precipitated with heptane to afford the title compound as a greenish-brown foam which was used in subsequent steps without further purification.

### Step B: 2-butyl-4-chloro-1-{[2′-(2-trityl-2H-tetrazol-5-yl)biphenyl-4-yl]methyl}-1H-imidazole-5-carboxylic acid

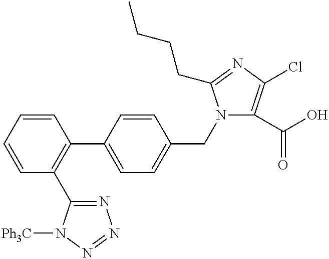

To a solution of E3174 (234.58 g, 0.54 mol) in DCM (4500 mL) was added triethylamine (85 mL, 0.59 mol) followed by a solution of trityl chloride (159 g, 0.56 mol) in DCM (800 mL) and the reaction mixture was stirred at rt overnight. The reaction mixture was washed with water, dried (MgSO₄), filtered, and concentrated under reduced pressure. Chromatography over silica gel eluting with acetone/heptane 20:80% afforded the title compound as an orange solid.

### Preparation of (3R,3aR,6R,6aR)-6-((1-chloroethoxy)carbonyloxy)hexahydrofuro[3,2-b]furan-3-yl 5,6-bis(nitrooxy)hexanoate

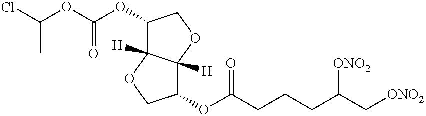

### Step A′: (3R,3aR,6R,6aR)-6-hydroxyhexahydrofuro[3,2-b]furan-3-yl hept-6-enoate

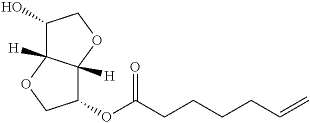

To a solution of 1,4:3,6-dianhydro-D-mannitol (3.00 g, 20.5 mmol), 4-dimethylaminopyridine (0.500 g, 4.11 mmol) and 5-hexenoic acid (2.34 g, 20.5 mmol) in DCM (60 ml), EDAC (5.90 g, 30.8 mmol) was added; the mixture was stirred at room temperature for 18 hrs. Then it was diluted with DCM and washed with water (2×40 ml). The organic layer was dried over sodium sulfate, filtered, concentrated and purified by flash chromatography (Biotage SP1, EtOAc/n-hexane from 20 to 80%), affording the title compound.

### Step B′: (3R,3aR,6R,6aR)-6-hydroxyhexahydrofuro[3,2-b]furan-3-yl 5,6-bis(nitrooxy)hexanoate

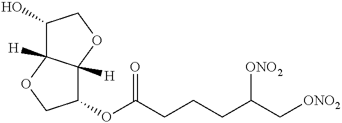

To an acetonitrile (45 ml) solution of (3R,3aR,6R,6aR)-6-hydroxyhexahydrofuro[3,2-b]furan-3-yl hept-6-enoate (2.05 g, 8.46 mmol) at −20° C. was added silver nitrate (1.72 g, 10.1 mmol) and iodine (2.58 g, 10.6 mmol). The mixture was stirred at −20° C. for 10 minutes. Silver nitrate was added (3.59 g, 21.1 mmol) and the mixture was heated in a microwave apparatus (40 minutes, 120° C.). The silver salts were filtered off and the solution was concentrated. The residue was purified by flash chromatography (Biotage SP1, EtOAc/n-hexane from 30 to 70%), affording the title compound as colorless oil.

### (Step C′: (3R,3aR,6R,6aR)-6-((1-chloroethoxy)carbonyloxy)hexahydrofuro[3,2-b]furan-3-yl 5,6-bis(nitrooxy)hexanoate

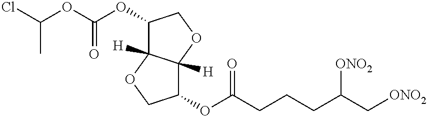

To a solution of (3R,3aR,6R,6aR)-6-hydroxyhexahydrofuro[3,2-b]furan-3-yl 5,6-bis(nitrooxy)hexanoate (0.650 g, 1.77 mmol) in DCM (5 ml), 1-chloroethyl chloroformate (0.249 ml, 2.30 mmol) and pyridine (0.185 ml, 2.30 mmol) were added; the mixture was stirred at room temperature for 18 hrs. Then the mixture was diluted with DCM, washed with a 5% solution of sodium dihydrogen phosphate (2×10 ml) and the organic layer was dried over sodium sulphate, filtered and concentrated under reduced pressure, affording the title compound.

### Step C: 1-(((3R,3aR,6R,6aR)-6-(5,6-bis(nitrooxy)hexanoyloxy)hexahydrofuro[3,2-b]furan-3-yloxy)carbonyloxy)ethyl 2-butyl-4-chloro-1-((2′-(1-trityl-1H-tetrazol-5-yl)biphenyl-4-yl)methyl)-1H-imidazole-5-carboxylate

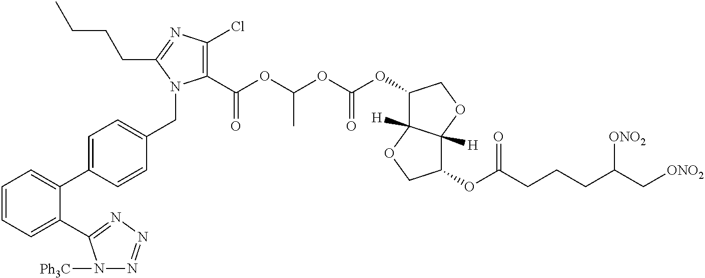

(3R,3aR,6R,6aR)-6-((1-chloroethoxy)carbonyloxy)hexahydrofuro[3,2-b]furan-3-yl 5,6-bis(nitrooxy)hexanoate (0.780 g, 1.65 mmol) was added to a stirred solution of 2-butyl-4-chloro-1-{[2′-(2-trityl-2H-tetrazol-5-yl)biphenyl-4-yl]methyl}-1H-imidazole-5-carboxylic acid (1.35 g, 1.98 mmol) and Cs₂CO₃ (0.645 g, 1.98 mmol) in DMF (10 ml). The solution was stirred at room temperature for 3 days. Then the mixture was diluted with EtOAc and washed with a 5% solution of sodium dihydrogen phosphate (2×30 ml) and water (1×30 ml). The organic layer was dried over sodium sulfate, filtered, concentrated and purified by flash chromatography (Biotage SP1, EtOAc/n-hexane from 20 to 80%), affording the title compound.

### Step D: 1-(((3R,3aR,6R,6aR)-6-(5,6-bis(nitrooxy)hexanoyloxy)hexahydrofuro[3,2-b]furan-3-yloxy)carbonyloxy)ethyl 1-((2′-(1H-tetrazol-5-yl)biphenyl-4-yl)methyl)-2-butyl-4-chloro-1H-imidazole-5-carboxylate

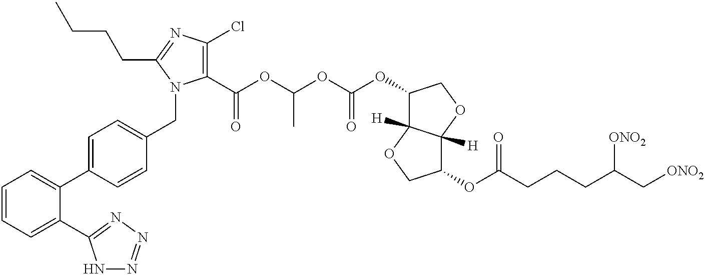

1-(((3R,3aR,6R,6aR)-6-(5,6-bis(nitrooxy)hexanoyloxy)hexahydrofuro[3,2-b]furan-3-yloxy)carbonyloxy)ethyl 2-butyl-4-chloro-1-((2′-(1-trityl-1H-tetrazol-5-yl)biphenyl-4-yl)methyl)-1H-imidazole-5-carboxylate (1.05 g, 0.918 mmol) was dissolved in 30 ml of MeOH and heated in a microwave apparatus (40 minutes, 70° C.). Then the solution was concentrated under reduced pressure and the residue purified by flash chromatography (Biotage SP1, MeOH/DCM from 0 to 5%), affording the title compound as white solid.

¹H-NMR (300 MHz, CDCl₃): 7.7-7.5 (4H, m); 7.1-6.96 (4H, m); 6.77 (1H, q); 5.61-5.39 (3H, m); 4.0-3.85 (3H, m); 4.75-4.55 (3h; Mm); 4.0-8 (2H, m); 3.75-3.05 (1H, m); 2.62 (2H, t); 2.45-2.35 (2H, m); 1.7-1.4 (9H, m); 1.35-1.19 (2H, m); 0.7 (3H, t).

### Example 2

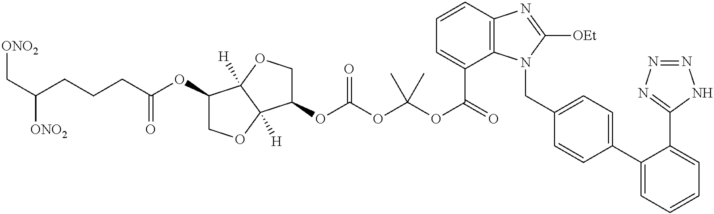

### 2-(((3R,3aR,6R,6aR)-6-(5,6-bis(nitrooxy)hexanoyloxy)hexahydrofuro[3,2-b]furan-3-yloxy)carbonyloxy)propan-2-yl 1-((2′-(1H-tetrazol-5-yl)biphenyl-4-yl)methyl)-2-ethoxy-1H-benzo[d]imidazole-7-carboxylate

### Step A: 1-methyl-1-{[(4-nitrophenoxy)carbonyl]oxy}ethyl 2-ethoxy-1-{[2′-(1-trityl-1H-tetrazol-5-yl)biphenyl-4-yl]methyl}-1H-benzimidazole-7-carboxylate

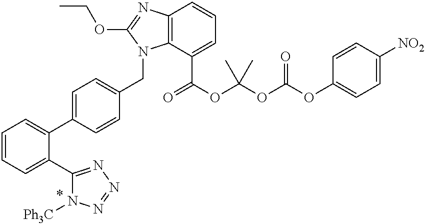

An orange suspension of mercuric oxide (1.17 g, 5.39 mmol) and 2-ethoxy-1-{[2′-(1-trityl-1H-tetrazol-5-yl)biphenyl-4-yl]methyl}-1H-benzimidazole-7-carboxylic acid (7.36 g, 10.8 mmol) in dry tetrahydrofuran (95 mL) was stirred at room temperature for 24 hrs. Then 2-chloroisopropyl p-nitrophenyl carbonate (prepared as described in U.S. Pat. No. 5,684,018) (1.40 g, 5.39 mmol) was added, and the reaction was stirred at room temperature for about 7 days and monitored by TLC (hexane:EtOAc 6:4). The mixture was diluted with DCM, washed with water, and the organic layer was dried over sodium sulfate and concentrated under reduced pressure. The residue was purified by flash chromatography (Biotage SP1, EtOAc/n-hexane from 7 to 60%), affording the title product.

¹H-NMR (300 MHz, CDCl₃): δ 8.23 (2H, d), 7.89-7.83 (1H, m), 7.80 (1H, d), 7.64 (1H, d), 7.51-7.40 (2H, m), 7.38-7.15 (13H, m), 7.02-6.88 (8H, m), 6.74 (1H, d), 5.63 (2H, s), 4.65 (2H, q), 1.83 (6H, s), 1.44 (3H, t).

### Step B: 2-(((3R,3aR,6R,6aR)-6-(5,6-bis(nitrooxy)hexanoyloxy)hexahydrofuro[3,2-b]furan-3-yloxy)carbonyloxy)propan-2-yl 2-ethoxy-1-((2′-(1-trityl-1H-tetrazol-5-yl)biphenyl-4-yl)methyl)-1H-benzo[d]imidazole-7-carboxylate

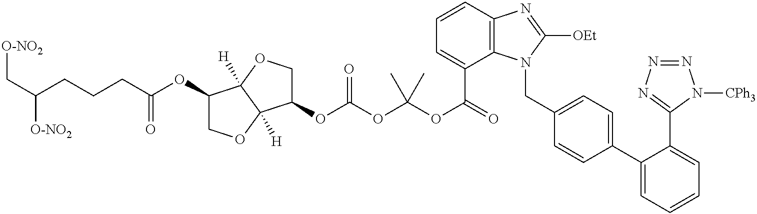

1-methyl-1-{[(4-nitrophenoxy)carbonyl]oxy}ethyl 2-ethoxy-1-{[2′-(1-trityl-1H-tetrazol-5-yl)biphenyl-4-yl]methyl}-1H-benzimidazole-7-carboxylate (1.35 g, 1.49 mmol) was dissolved in DCM (50 ml) then (3R,3aR,6R,6aR)-6-hydroxyhexahydrofuro[3,2-b]furan-3-yl 5,6-bis(nitrooxy)hexanoate (prepared as described in EXAMPLE 1, Step B′) (0.50 g, 1.36 mmol), dimethylaminopyridine (0.25 g, 2.04 mmol) and scandium trifluoromethanesulfonate (0.13 g, 0.27 mmol) were added. After being stirred at room temperature for 24 hrs, the reaction was washed with a 5% solution of sodium dihydrogen phosphate. The organic extract was dried over sodium sulfate and the solvent was evaporated under reduced pressure. The residue was purified by flash chromatography (Biotage SP1, EtOAc/n-hexane from 40 to 90%), affording the title compound as a yellow solid.

¹H-NMR (300 MHz, CDCl₃): δ 7.89 (1H, d), 7.78 (1H, m), 7.66 (1H, d), 7.52-7.40 (2H, m), 7.39-7.15 (10H, m), 7.05-6.90 (8H, m), 6.72 (2H, m), 6.74 (1H, d), 5.63 (2H, s), 5.30 (1H, m), 5.10-4.90 (2H, m), 4.80-4.60 (5H, m), 4.52-4.40 (1H, m), 4.05-3.95 (2H, m), 3.90-3.75 (2H, m), 2.50-2.40 (2H, m), 1.90-1.70 (10H, m), 1.42 (3H, t).

### Step C: 2-(((3R,3aR,6R,6aR)-6-(5,6-bis(nitrooxy)hexanoyloxy)hexahydrofuro[3,2-b]furan-3-yloxy)carbonyloxy)propan-2-yl 1-((2′-(1H-tetrazol-5-yl)biphenyl-4-yl)methyl)-2-ethoxy-1H-benzo[d]imidazole-7-carboxylate

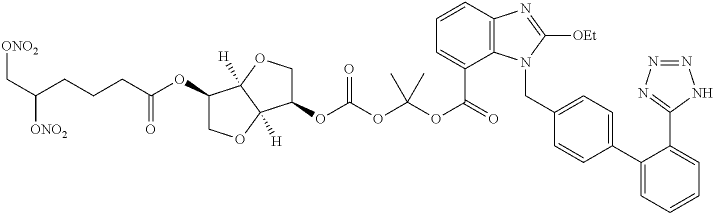

2-(((3R,3aR,6R,6aR)-6-(5,6-bis(nitrooxy)hexanoyloxy)hexahydrofuro[3,2-b]furan-3-yloxy)carbonyloxy)propan-2-yl 2-ethoxy-1-((2′-(1-trityl-1H-tetrazol-5-yl)biphenyl-4-yl)methyl)-1H-benzo[d]imidazole-7-carboxylate (0.40 g, 0.35 mmol) was dissolved in a very small amount of DCM (enough to make the dissolution), then methanol (50 ml) was added. After stirring and heating at 45° C. for 48 hrs, the solvent was removed under reduced pressure and the residue was purified by flash chromatography (Biotage SP1, MeOH/DCM from 0.5 to 4%), affording the title compound as a white solid.

¹H-NMR (300 MHz, DMSO-d₆): δ 8.0 (1H, d); 7.62 (2H, m); 7.51 (1H, d); 7.31 (2H, m); 7.69-7.66 (6H, m); 5.61 (2H, s); 5.31 (1H, m); 5.05 (1H, m); 4.91 (1H, m); 4.76-4.74 (1H, dd); 4.65 (2H, m); 4.51 (1H, dd); 4.26 (2H, m); 3.98 (2H, m); 3.77 (2H, m); 2.45 (2H, m); 1.82 (4H, m); 1.67 (3H, s); 1.62 (3H, s); 1.40 (3H, t).

### Intermediate 1

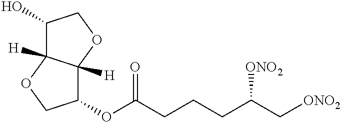

### (S)-((3R,3aR,6R,6aR)-6-hydroxyhexahydrofuro[3,2-b]furan-3-yl) 5,6-bis(nitrooxy)hexanoate

### Step A: tert-butyl hex-5-enoate

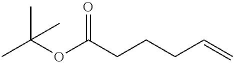

To a solution of 5-hexenoic acid (15.2 mL, 0.131 mol) in DCM (375 mL), cooled to 0° C., tert-butanol (176 mL, 1.84 mol) and then 4-dimethylaminopyridine (3.21 g, 26.3 mmol) were added. The mixture was stirred at room temperature for 22 hrs, filtered and concentrated. The residue was redissolved in DCM/n-hexane and concentrated under reduced pressure. The crude oil was purified by flash chromatography (Biotage SP I, EtOAc/n-hexane from 5 to 10%), affording the title product.

### Step B: (S)-tert-butyl 5,6-dihydroxyhexanoate

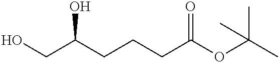

To a suspension of AD-mix alpha (70 g) in water/butanol 1:1 (512 mL) cooled to 0° C., tert-butyl hex-5-enoate (8.5 g, 49.92 mmol) was added. The reaction mixture was stirred at 4° C. for 70 hrs. Then the reaction mixture was cooled to 0° C. and EtOAc (280 mL), followed by continuous portionwise addition of sodium metabisulfite (20.6 g). The mixture was stirred for 30 min at 0° C. and at rt for 1 hour. The organic layer was separated and the aqueous phase extracted with ethyl acetate. The combined organic layers were dried over sodium sulfate and concentrated under reduced pressure. Chromatography over silica gel eluting with EtOAc 100% afforded the title compound as a pale yellow oil.

### Step C: (S)-tert-butyl 5,6-di(nitrooxy)hexanoate

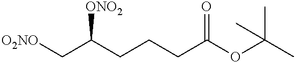

To a solution of fuming nitric acid (10.25 mL, 247.22 mmol) and acetic anhydride (37.6 mL) cooled to 0° C., a solution of (S)-tert-butyl 5,6-dihydroxyhexanoate (10.1 g, 49.44 mmol) in DCM (10 mL) was added dropwise. The reaction mixture was stirred at 0° C. for 1 hour, adjusted to pH 7 by addition of aqueous NaOH and extracted with DCM. The organic layer was separated and the aqueous phase extracted with ethyl acetate. The combined organic layers were dried over sodium sulfate and concentrated under reduced pressure. The crude residue was purified by flash chromatography (Biotage SP1, EtOAc/n-hexane from 10 to 50%), affording the title product as a pale yellow oil.

### Step D: (S)-5,6-di(nitrooxy)hexanoic acid

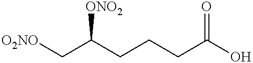

To a solution of (S)-tert-butyl 5,6-di(nitrooxy)hexanoate (12.41 g, 42.155 mmol) in DCM (47 mL) cooled to 0° C. under N₂, boron trifluoride diethyl ether complex (5.82 mL, 46.37 mmol) was added. The reaction mixture was stirred at 0° C. for 10 minutes and at rt for 3 hrs. The solution was washed with brine, the organic layer was separated, dried over sodium sulfate and concentrated under reduced pressure. The crude brown oil was used in the next step without further purification.

### Step E: (3R,3aR,6R,6aR)-6-(tetrahydro-2H-pyran-2-yloxy)hexahydrofuro[3,2-b]furan-3-ol

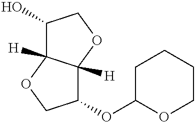

To a solution of 1,4:3,6-dianhydro-D-mannitol (5.00 g, 34.2 mmol) in DCM (102 mL) 3,4-dihydro-2H-pyran (3.88 mL, 42.8 mmol) was added, followed by p-toluensulfonic acid (65 mg, 1.34 mmol). The reaction mixture was stirred at rt for 16 hrs. The solution was washed with brine, the organic layer was separated, dried over sodium sulfate and concentrated under reduced pressure. The crude residue was purified by flash chromatography (Biotage SP1, EtOAc/n-hexane from 30 to 100%), affording the title product as pale yellow oil.

### Step F: (5S)-((3R,3aR,6R,6aR)-6-(tetrahydro-2H-pyran-2-yloxy)hexahydrofuro[3,2-b]furan-3-yl)5,6-bis(nitrooxy)hexanoate

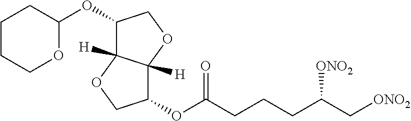

To a solution of (3R,3aR,6R,6aR)-6-(tetrahydro-2H-pyran-2-yloxy)hexahydrofuro[3,2-b]furan-3-ol (2.96 g, 12.9 mmol) in DCM (40.8 mL) a solution of the crude (S)-5,6-di(nitrooxy)hexanoic acid (3.06 g, 12.85 mmol) (Step D) in DCM (9.15 mL) was added followed by EDAC (3.69 g, 19.28 mmol) and 4-dimethylaminopyridine (175 mg, 1.28 mmol). The reaction mixture was stirred at rt for 16.5 hrs. The solvent was removed under reduced pressure. The crude residue was purified by flash chromatography (Biotage SP1, EtOAc/n-hexane from 10 to 60%), affording the title product as pale yellow oil.

### Step G: (S)-((3R,3aR,6R,6aR)-6-hydroxyhexahydrofuro[3,2-b]furan-3-yl) 5,6-bis(nitrooxy)hexanoate

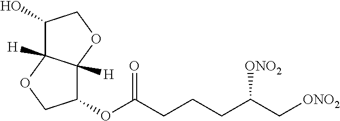

To a solution of (5S)-((3R,3aR,6R,6aR)-6-(tetrahydro-2H-pyran-2-yloxy)hexahydrofuro[3,2-b]furan-3-yl) 5,6-bis(nitrooxy)hexanoate (2.27 g, 5.04 mmol) in ethanol (40 mL) pyridinium p-toluensulfonate (127 mg, 0.504 mmol) was added The reaction mixture was stirred at 45° C. for 4 hrs. The reaction mixture was filtered, concentrated under reduced pressure. The crude residue was purified by flash chromatography (Biotage SP1, EtOAc/n-hexane from 10 to 60%), affording the title product as pale yellow oil.

¹H-NMR ((300 MHz, CDCl₃): δ 5.30 (1H, dd, 5.16 (1H, q), 4.77 (1H, d), 4.71 (1H, t), 4.50 (2H, m), 4.26 (1H, m), 4.12 (1H, dd), 3.94 (1H, dd), 3.85 (1H, dd), 3.58 (1H, dd), 2.61 (1H, d), 2.47 (2H, m), 1.83 (4H, m).

### Intermediate 2

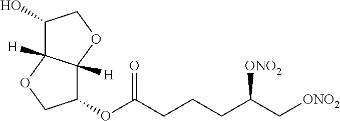

### (R)-((3R,3aR,6R,6aR)-6-hydroxyhexahydrofuro[3,2-b]furan-3-yl) 5,6-bis(nitrooxy)hexanoate

The title compound was prepared by following the procedure for the synthesis of Intermediate 1, except that in Step C the reagent ADmix-alpha was replaced by ADmix-beta.

¹H-NMR (300 MHz, CDCl₃): δ 5.38-5.26 (1H, m), 5.18 (1H, q), 4.81-4.70 (2H, m), 4.54-4.46 (2H, m), 4.32 (1H, q), 416-4.10 (1H, m), 4.10-3.85 (2H, m), 3.62-3.55 (1H, m), 2.58 (1H, d), 1.90-1.77 (4H, m).

### Intermediate 3

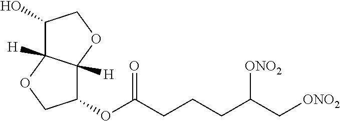

### 6-((3R,3aR,6R,6aR)-6-hydroxyhexahydrofuro[3,2-b]furan-3-yloxy)hexane-1,2-diyl dinitrate

### Step A: (3R,3aR,6R,6aR)-6-(hex-5-enyloxy)hexahydrofuro[3,2-b]furan-3-ol

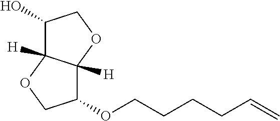

To a solution of 1,4:3,6-dianhydro-D-mannitol (4.00 g, 27.4 mmol) and Cs₂CO₃ (20.0 g, 60.2 mmol) in DMF (60 ml), 6-bromo-1-hexene (5.5 ml, 41.1 mmol), was added; the mixture was stirred at room temperature for 18 hrs. Then it was diluted with EtOAc and washed with a 5% solution of sodium dihydrogen phosphate (2×40 ml) and water (2×40 ml). The organic layer was dried over sodium sulfate, filtered, concentrated and purified by flash chromatography (Biotage SP1, EtOAc/n-hexane from 20 to 80%), affording the title compound.

### Step B: 6-((3R,3aR,6R,6aR)-6-hydroxyhexahydrofuro[3,2-b]furan-3-yloxy)hexane-1,2-diyl dinitrate

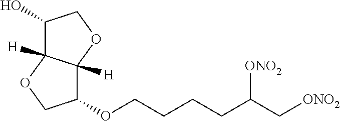

To an acetonitrile (32 ml) solution of (3R,3aR,6R,6aR)-6-(hex-5-enyloxy)hexahydrofuro[3,2-b]furan-3-ol (1.35 g, 5.91 mmol) at −20° C. was added silver nitrate (1.21 g, 7.12 mmol) and iodine (1.80 g, 7.10 mmol). The mixture was stirred at −20° C. for 10 minutes. Silver nitrate was added (2.51 g, 14.8 mmol) and the mixture was heated in a microwave apparatus (40 minutes, 120° C.). The silver salts were filtered off and the solution was concentrated. The residue was purified by flash chromatography (Biotage SP1, EtOAc/n-hexane from 30 to 100%), affording the title compound as colorless oil.

¹H-NMR (300 MHz, CDCl₃): δ 5.35-5.27 (1H, m), 4.76 (1H, dd), 4.58-4.45 (3H, m), 4.35-4.25 (1H, m), 4.12-3.95 (3H, m), 3.75-3.65 (3H, m), 3.55-3.48 (1H, m), 2.86 (1H, d), 1.85-1.50 (6H, m).

### Intermediate 4

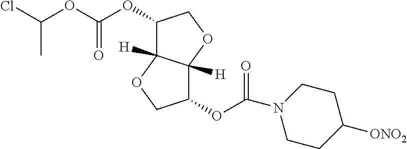

### (3R,3aR,6R,6aR)-6-(1-chloroethoxy)carbonyloxy)hexahydrofuro[3,2-b]furan-3-yl 4-(nitrooxy)piperidine-1-carboxylate

### Step A: tert-butyl 4-(nitrooxy)piperidine-1-carboxylate

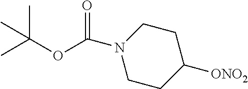

To a solution of tert-butyl 4-hydroxy-1-piperidinecarboxylate (2.00 g, 9.94 mmol), tetraethylammonium nitrate (3.82 g, 19.9 mmol) and 2,6-di-tert-butyl-4-methylpyridine (5.10 g, 24.9 mmol) in DCM (190 ml) cooled to −70° C. and under nitrogen, a solution of trifluoromethansulfonic anhydride (1.8 ml, 10.9 mmol) in DCM (62 ml) was added dropwise. The resulting mixture was stirred for 3 hrs at −65° C. Then the mixture was slowly warmed to room temperature, diluted with DCM and washed with 5% aqueous sodium dihydrogen phosphate. The organic layer was dried over sodium sulfate, filtered and concentrated, affording the title compound which was used in subsequent steps without further purification

### Step B: Piperidin-4-yl nitrate hydrochloride

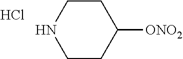

To a solution of tert-butyl 4-(nitrooxy)piperidine-1-carboxylate (2.10 g; 8.53 mmol) in DCM (15 ml) cooled to 0° C., HCl gas was bubbled for 2 hrs. The solvent was concentrated and the residue was treated with diethyl ether, affording the title compound which was used in subsequent steps without further purification.

### Step C: (3R,3aR,6R,6aR)-6-hydroxyhexahydrofuro[3,2-b]furan-3-yl 4-nitrophenyl carbonate

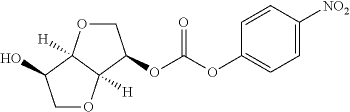

To a solution of 1,4:3,6-dianhydro-D-mannitol (3.00 g, 20.5 mmol) and triethylamine (3.15 ml, 22.6 mmol) in DCM (100 ml), 4-nitrophenyl chloroformate (4.55 g, 22.6 g) was added and the mixture was stirred at room temperature for 18 hrs. Then the mixture was washed with a 5% solution of sodium dihydrogen phosphate (2×50 ml). The organic layer was dried over sodium sulfate, filtered, concentrated and purified by flash chromatography (Biotage SP1, EtOAc/n-hexane from 20 to 80%), affording the title compound.

### Step D: (3R,3aR,6R,6aR)-6-hydroxyhexahydrofuro[3,2-b]furan-3-yl 4-(nitrooxy)piperidine-1-carboxylate

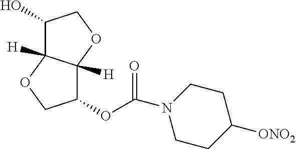

To a solution of piperidin-4-yl nitrate hydrochloride (0.620 g, 3.40 mmol), triethylamine (0.567 ml, 4.07 mmol) and 4-dimethylaminopyridine (0.083 g, 0.680 mmol) in DCM (23 ml), (3R,3aR,6R,6aR)-6-hydroxyhexahydrofuro[3,2-b]furan-3-yl 4-nitrophenyl carbonate (1.06 g, 3.40 mmol) was added; the mixture was stirred at room temperature for 18 hrs. Then the mixture was diluted with DCM and washed with a 5% solution of sodium dihydrogen phosphate (2×30 ml) and brine (1×30 ml). The organic layer was dried over sodium sulfate, filtered, concentrated and purified by flash chromatography (Biotage SP1, EtOAc/n-hexane from 30 to 100%), affording the title compound as a yellow oil.

### Step E: (3R,3aR,6R,6aR)-6-((1-chloroethoxy)carbonyloxy)hexahydrofuro[3,2-b]furan-3-yl 4-(nitrooxy)piperidine-1-carboxylate

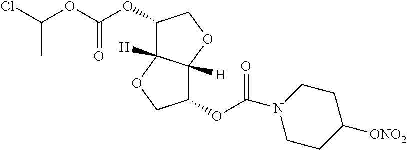

To a solution of (3R,3aR,6R,6aR)-6-hydroxyhexahydrofuro[3,2-b]furan-3-yl 4-(nitrooxy)piperidine-1-carboxylate (1.00 g, 3.14 mmol) in DCM (10 ml), 1-chloroethyl chloroformate (0.441 ml, 4.08 mmol) and pyridine (0.328 ml, 4.08 mmol) were added; the mixture was stirred at room temperature for 18 hrs. Then the mixture was diluted with DCM and washed with a 5% solution of sodium dihydrogen phosphate (2×10 ml). The organic layer was dried over sodium sulfate, filtered, concentrated and purified by flash chromatography (Biotage SP1, EtOAc/n-hexane from 30 to 100%), affording the title compound.

¹H-NMR (300 MHz, CDCl₃): δ 6.47-6.42 (1H, m), 5.20-5.02 (3H, m), 4.80-4.70 (2H, m), 4.12-4.00 (2H, m), 3.98-3.70 (4H, m), 3.50-3.26 (2H, m), 2.31-1.92 (2H, m), 1.90-1.71 (5H, m).

### Intermediate 5

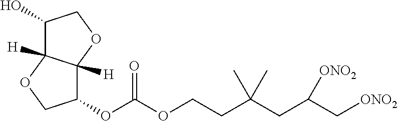

### 3,3-dimethyl-5,6-bis(nitrooxy)hexyl (3R,3aR,6R,6aR)-6-hydroxyhexahydrofuro[3,2-b]furan-3-yl carbonate

### Step A: 3,3-dimethyl-5,6-bis(nitrooxy)hexyl 4-nitrobenzoate

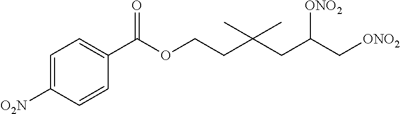

To an acetonitrile (60 ml) solution of 3,3-dimethylhex-5-enyl 4-nitrobenzoate (3.00 g, 10.8 mmol) at −20° C. was added silver nitrate (2.21 g, 13.0 mmol) and iodine (3.30 g, 13.0 mmol). The mixture was stirred at −20° C. for 10 minutes. Silver nitrate was added (4.6 g, 27.1 mmol) and the mixture was heated in a microwave apparatus (40 minutes, 120° C.). The silver salts were filtered off and the solution was concentrated. The residue was purified by flash chromatography (Biotage SP1, EtOAc/n-hexane from 20 to 80%), affording the title compound.

### Step B: 6-hydroxy-4,4-dimethylhexane-1,2-diyl dinitrate

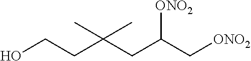

To a solution of 3,3-dimethyl-5,6-bis(nitrooxy)hexyl 4-nitrobenzoate (1.9 g, 4.73 mmol) in THF:EtOH 1:1 (12 ml) a aqueous solution of NaOH 3N (3.8 ml, 11.4 mmol) was added and the mixture was stirred at room temperature for 3 hrs. Then the mixture was diluted with a saturated solution of sodium bicarbonate and the product was extracted with EtOAc (3×30 ml). The organic layer was dried over sodium sulfate, filtered and concentrated under reduced pressure, affording the title compound which was used in the subsequent step without further purification.

### Step C: 3,3-dimethyl-5,6-bis(nitrooxy)hexyl (3R,3aR,6R,6aR)-6-hydroxyhexahydrofuro[3,2-b]furan-3-yl carbonate

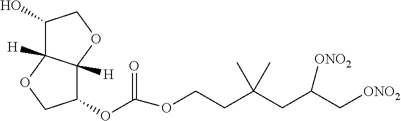

To a solution of (3R,3aR,6R,6aR)-6-hydroxyhexahydrofuro[3,2-b]furan-3-yl 4-nitrophenyl carbonate (0.962 g, 3.10 mmol) in DCM (40 ml), (prepared as described in INTERMEDIATE 4 Step C), 6-hydroxy-4,4-dimethylhexane-1,2-diyl dinitrate (0.780, 3.10 mmol) and 4-dimethylaminopyridine (0.379 g, 3.10 mmol) were added and the mixture was stirred at room temperature for 18 hrs. Then the mixture was diluted with DCM and washed with a 5% solution of sodium dihydrogen phosphate (2×30 ml) and brine (1×30 ml). The organic layer was dried over sodium sulfate, filtered and concentrated. The residue was purified by flash chromatography (Biotage SP1, EtOAc/n-hexane from 20 to 80%), affording the title compound.

¹H-NMR (300 MHz, CDCl₃): δ 5.52-5.43 (1H, m), 5.09 (1H, q), 4.80-4.71 (2H, m), 4.52-4.40 (2H, m), 4.37-4.21 (3H, m), 4.15-3.90 (3H, m), 3.58 (1H, t), 2.53 (1H, d), 1.82-1.55 (4H, m), 1.03 (6H, m).

### Intermediate 6

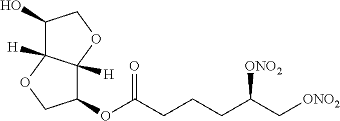

### (3S,3aR,6S,6aR)-6-(2-chloropropanoyloxy)hexahydrofuro[3,2-b]furan-3-yl 5R,6-bis(nitrooxy)hexanoate

### Step A: (3S,3aR,6S,6aR)-hexahydrofuro[3,2-b]furan-3,6-diyl dibenzoate

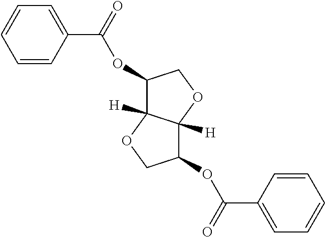

1,4:3,6-dianhydro-D-mannitol (3.00 g, 20.53 mmol) was dissolved in THF (40 ml). The solution was cooled (0° C.), then triphenylphosphine (11.85 g, 45.17 mmol), benzoic acid (5.52 g, 45.17 mmol) and diisopropyl azodicarboxylate (8.75 ml, 45.17 mmol) were added. The reaction was allowed to warm to room temperature and stirred for 12 hrs. Then the solvent was removed under reduced pressure and the residue was purified by flash chromatography (Biotage SP1, EtOAc/n-hexane from 10 to 30%), affording of the title compound as a white solid.

### Step B: (3S,3aR,6S,6aR)-hexahydrofuro[3,2-b]furan-3,6-diol

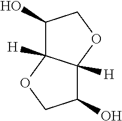

To (3S,3aR,6S,6aR)-hexahydrofuro[3,2-b]furan-3,6-diyl dibenzoate (6.30 g, 17.8 mmol), a mix of NaOH 10%:MeOH=1:1 (60 ml) was added. After stirring at room temperature for 24 hrs, the solvent was evaporated under reduced pressure. Water (30 ml) was added and extraction with EtOAc (30 ml) was carried out. The aqueous layer (pH adjusted to 6 with H₃PO₄ 5%) was evaporated under reduced pressure to leave a white residue. Tetrahydrofuran was added in a large amount, then the suspension was filtered and the filtrate was concentrated to give the title product as a white solid.

### Step C: (3S,3aR,6S,6aR)-6-(tetrahydro-2H-pyran-2-yloxy)hexahydrofuro[3,2-b]furan-3-ol

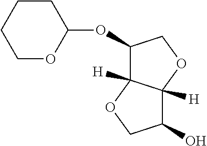

To a solution of (3S,3aR,6S,6aR)-hexahydrofuro[3,2-b]furan-3,6-diol (0.57 g, 3.90 mmol) and 3,4-dihydro-2H-pyran (0.41 g, 4.88 mmol) in DCM (15 ml), toluene-4-solfonic acid monohydrate (0.0074 g, 0.039 mmol) was added and the mixture was stirred at room temperature for 3 hrs. Then the reaction mixture was washed with a saturated solution of NaHCO₃ and brine. The organic extract was dried over sodium sulfate and the solvent was evaporated under reduced pressure. The residue was purified by flash chromatography (Biotage SP1, EtOAc/n-hexane from 30 to 100%), affording the title product as a white solid.

### Step D: (5R)-((3S,3aR,6S,6aR)-6-(tetrahydro-2H-pyran-2-yloxy)hexahydrofuro[3,2-b]furan-3-yl) 5,6-bis(nitrooxy)hexanoate

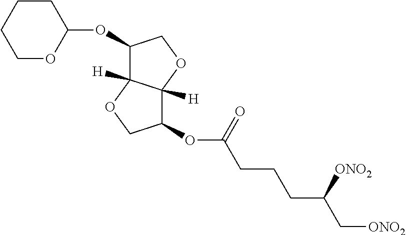

3S,3aR,6S,6aR)-6-(tetrahydro-2H-pyran-2-yloxy)hexahydrofuro[3,2-b]furan-3-ol (0.64 g, 2.80 mmol) and (5R)-5,6-bis-nitrooxy-hexanoic acid (1.18 g, 4.95 mmol) (obtained as described in INTERMEDIATE 2) were dissolved in DCM (30 ml), EDAC (0.81 g, 4.20 mmol) and 4-dimethylaminopyridine (0.068 g, 0.56 mmol) were added. After stirring at room temperature for 12 hrs, the reaction mixture was washed with water. The organic layer was dried over sodium sulfate, and evaporated under reduced pressure. The residue was purified by flash chromatography (Biotage SP1, EtOAc/n-hexane from 10 to 80%), affording the title product as an oil.

### Step E: (3S,3aR,6S,6aR)-6-(2-chloropropanoyloxy)hexahydrofuro[3,2-b]furan-3-yl 5R,6-bis(nitrooxy)hexanoate

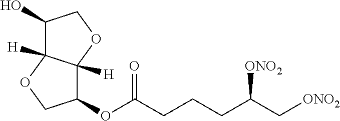

The title compound was obtained following the procedure described in INTERMEDIATE 1 Step G.

¹H-NMR (300 MHz, CDCl₃): δ 5.33 (1H, m), 5.20 (1H, m), 4.80-4.74 (1H, m), 7.67 (1H, d), 4.57-4.47 (2H, m), 4.39 (1H, s), 4.00-3.85 (4H, m), 2.48-2.39 (2H, m), 1.97 (1H, d), 1.90-1.73 (4H, m).

### Example 3

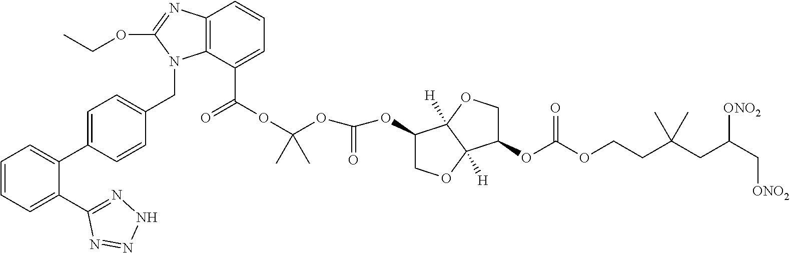

### 2-(((3R,3aR,6R,6aR)-6-((3,3-dimethyl-5,6-bis(nitrooxy)hexyloxy)carbonyloxy)hexahydro furo[3,2-b]furan-3-yloxy)carbonyloxy)propan-2-yl 1-((2′-(2H-tetrazol-5-yl)biphenyl-4-yl)methyl)-2-ethoxy-1H-benzo[d]imidazole-7-carboxylate

The title compound was synthesized by following the procedure reported in EXAMPLE 2, except that (3R,3aR,6R,6aR)-6-hydroxyhexahydrofuro[3,2-b]furan-3-yl 5,6-bis(nitrooxy)hexanoate was replaced by 3,3-dimethyl-5,6-bis(nitrooxy)hexyl (3R,3aR,6R,6aR)-6-hydroxyhexahydrofuro[3,2-b]furan-3-yl carbonate (INTERMEDIATE 5).

¹H-NMR (300 MHz, CDCl₃): δ 8.00 (1H, d), 7.62-7.54 (2H, m), 7.51 (1H, d), 7.32-7.28 (1H, m), 6.95-6.67 (6H, m), 5.61 (2H, s), 5.50-5.42 (1H, m), 5.00-4.85 (2H, m), 4.80-4.63 (3H, m), 4.47-4.38 (1H, m), 4.33-4.15 (4H, m), 4.03-3.95 (2H, m), 3.86-3.75 (2H, m), 1.80-1.52 (10H, m), 1.43 (3H, t), 1.02 (6H, s).

### Example 4

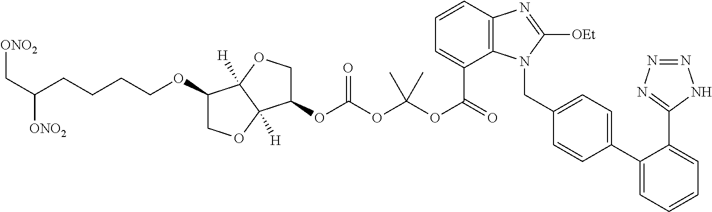

### 2-(((3R,3aR,6R,6aR)-6-(5,6-bis(nitrooxy)hexyloxy)hexahydrofuro[3,2-b]furan-3-yloxy)carbonyloxy)propan-2-yl 2-ethoxy-1-((2′-(1-trityl-1H-tetrazol-5-yl)biphenyl-4-yl)methyl)-1H-benzo[d]imidazole-7-carboxylate

Title compound was synthesized by following the procedure reported in EXAMPLE 2, except that the reagent that (3R,3aR,6R,6aR)-6-hydroxyhexahydrofuro[3,2-b]furan-3-yl 5,6-bis(nitrooxy)hexanoate was replaced by 6-((3R,3aR,6R,6aR)-6-hydroxyhexahydrofuro[3,2-b]furan-3-yloxy)hexane-1,2-diyl dinitrate (INTERMEDIATE 3).

¹H-NMR (300 MHz, CDCl₃): δ 8.00 (1H, d), 7.62-7.50 (3H, m), 7.34-7.28 (1H, m), 6.99-6.82 (4H, m), 6.77-6.68 (2H, m), 5.70-5.55 (2H, m), 5.35-5.25 (1H, m), 5.00-4.88 (1H, m), 4.80-4.62 (2H, m), 4.52-4.44 (2H, m), 4.40-4.25 (2H, m), 4.04-3.85 (3H, m), 3.68-3.42 (2H, m), 1.85-1.40 (15H, m).

### Example 5

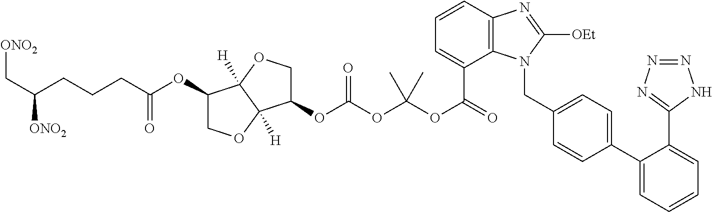

### 2-(((3R,3aR,6R,6aR)-6-((R)-5,6-bis(nitrooxy)hexanoyloxy)hexahydrofuro[3,2-b]furan-3-yloxy)carbonyloxy)propan-2-yl 1-((2′-(1H-tetrazol-5-yl)biphenyl-4-yl)methyl)-2-ethoxy-1H-benzo[d]imidazole-7-carboxylate

Title compound was synthesized by following the procedure reported in EXAMPLE 2, except that the reagent (3R,3aR,6R,6aR)-6-hydroxyhexahydrofuro[3,2-b]furan-3-yl 5,6-bis(nitrooxy)hexanoate was replaced by (R)-(3R,3aR,6R,6aR)-6-hydroxyhexahydrofuro[3,2-b]furan-3-yl) 5,6-bis(nitrooxy)hexanoate (INTERMEDIATE 2).

¹H-NMR (300 MHz, DMSO-d₆): δ 7.71-7.43 (6H, m), 7.21 (1H, t), 7.00 (2H, d), 6.84 (2H, d), 5.53 (2H, s), 5.43 (1H, m), 5.02-4.89 (3H, m), 4.75-4.53 (5H, m), 3.96-3.85 (2H, m), 3.73-3.58 (2H, m), 2.39 (2H, t), 1.76-1.58 (10H, m), 1.40 (3H, t).

### Example 6

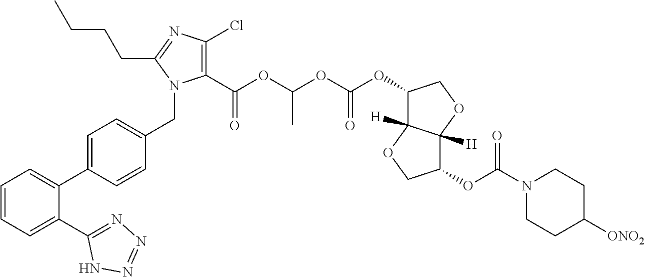

### (3R,3aR,6R,6aR)-6-(1-(1-((2′-(1H-tetrazol-5-yl)biphenyl-4-yl)methyl)-2-butyl-4-chloro-1H-imidazole-5-carbonyloxy)ethoxy)carbonyloxy)hexahydrofuro[3,2-b]furan-3-yl 4-(nitrooxy)piperidine-1-carboxylate

Title compound was synthesized by following procedure reported in EXAMPLE 1 except that the reagent (3R,3aR,6R,6aR)-6-((1-chloroethoxy)carbonyloxy)hexahydrofuro[3,2-b]furan-3-yl 5,6-bis(nitrooxy)hexanoate was replaced by (3R,3aR,6R,6aR)-6-((1-chloroethoxy)carbonyloxy)hexahydrofuro[3,2-b]furan-3-yl 4-(nitrooxy)piperidine-1-carboxylate

### Intermediate 4

¹H-NMR (300 MHz, DMSO-d₆): δ 7.71-7.50 (4H, m), 7.12-7.05 (2H, m), 7.01-6.92 (2H, m), 6.81-6.70 (1H, m), 5.61-5.45 (2H, m), 5.28-5.19 (1H, m), 5.00-4.89 (2H, m), 4.70-4.59 (1H, m), 4.58-4.50 (1H, m), 3.99-3.85 (2H, m), 3.78-3.58 (4H, m), 3.40-3.30 (2H, 2.63 (2H, t), 2.00-1.90 (2H, m), 1.70-1.42 (7H, m), 1.31-1.20 (3H, m), 0.82 (3H, t).

### Assay on Vascular Tone

The ability of the compounds of the invention to induce vasorelaxation in comparison to native angiotensin II receptor blockers (ARBs), was tested in vitro in isolated rabbit thoracic aorta preparations (Wanstall J. C. et al., Br. J. Pharmacol., 134:463-472, 2001). Male New Zealand rabbits were anaesthetized with thiopental-Na (50 mg/kg, iv), sacrificed by exsanguinations and then the thorax was opened and the aorta dissected. Aortic ring preparations (4 mm in length) were set up in physiological salt solution (PSS) at 37° C. in small organ chambers (5 ml). The composition of PSS was (mM): NaCl 130, NaHCO₃ 14.9, KH₂PO₄ 1.2, MgSO₄ 1.2, HEPES 10, CaCl₂, ascorbic acid 170 and glucose 1.1 (95% O₂/5% CO₂; pH 7.4). Each ring was mounted under 2 g passive tension. Isometric tension was recorded with a Grass transducer (Grass FT03) attached to a BIOPAC MP150 System. Preparations were allowed to equilibrate for 1 h, and then contracted submaximally with noradrenaline (NA, 1 μM) and, when the contraction was stable, acetylcholine (ACh, 10 μM) was added. A relaxant response to ACh indicated the presence of a functional endothelium. Vessels that were unable to contract NA or showed no relaxation to Ach were discarded. When a stable precontraction was reached, a cumulative concentration-response curve to either of the vasorelaxant agents was obtained in the presence of a functional endothelium. Each arterial ring was exposed to only one combination of inhibitor and vasorelaxant. Moreover, the effect of the soluble guanylyl cyclase inhibitor ODQ (1-H-(1,2,4)-oxadiazol(4,3-a)quinoxalin-1-one) on vasorelaxation elicited by the compounds was examined preincubating the aortic rings with ODQ (10 μM) for 20 min.

Responses to relaxing agents are expressed as a percentage of residual contraction and plotted against concentration of test compound. EC₅₀ values (where EC₅₀ is the concentration producing 50% of the maximum relaxation to the test compound) were interpolated from these plots.

During the experimental period, the plateau obtained with NA was stable without significant spontaneous loss of contraction in the aortic rings. Under these experimental conditions, the native ARBs did not produce relaxation at any of the concentration tested, the curve being not different from that built up in the presence of vehicle alone.

As shown in Table 1, the compounds of the invention were able to induce relaxation in a concentration-dependent manner. Furthermore, in experiments performed in the presence of ODQ (10 μM), the vasorelaxant responses to tested compounds were inhibited.

TABLE 1

| | Compound | EC₅₀ in vessel |
|---|---|---|
| Structure | Number | relaxation assay |
| 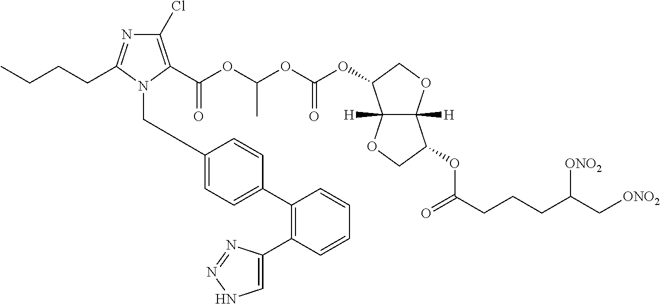 | Example 1 | 6.2 μM |
| 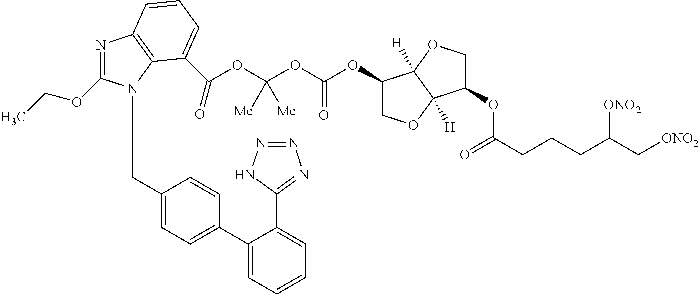 | Example 2 | 0.27 μM |
| 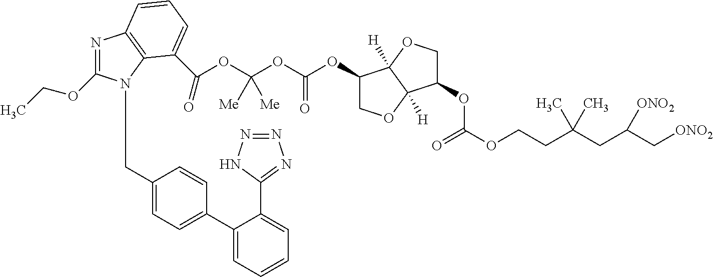 | Example 3 | 0.18 μM |

### Assay for Antihypertensive Activity (In Vivo).

The ability of the compounds of the invention to decrease blood pressure was evaluated in conscious spontaneously hypertensive rats (SHRs). SHRs (250-300 g) received a single oral dose of tested compounds. Systolic blood pressure (SBP) and heart rate were monitored by telemetry for 24 hrs after dosing. SBP was evaluated before (baseline) and at different time points (i.e. 2-6, 12, 21-24 hrs) following treatment by oral administration of the compounds. The data were processed both as the absolute value or as a delta between the absolute value and its own baseline.

The Dataquest IV telemetry system (Data Sciences International) was used for measurement of systolic pressure, diastolic pressure, mean arterial pressure, heart rate, and motor activity. The monitoring system consists of a transmitter (radio frequency transducer model TA11PA), receiver panel, consolidation matrix, and personal computer with accompanying software. Before the device was implanted, calibrations were verified to be accurate within ±3 mmHg. Rats were anesthetized with ketamine/xylazine/acepromazine, and the flexible catheter of the transmitter was surgically secured in the abdominal aorta just below the renal arteries. The transmitter was sutured subcutaneously. Rats were housed in individual cages after the operation. Each cage was placed over the receiver panel that was connected to the personal computer for data acquisition. The rats were unrestrained and free to move within their cages. Hemodynamic data were sampled every 2 minutes for 10 seconds.

Compared to Compound A (reference compound), the compounds of the invention provided BP lowering with extended peak effect and duration of action (see Data Table 2).

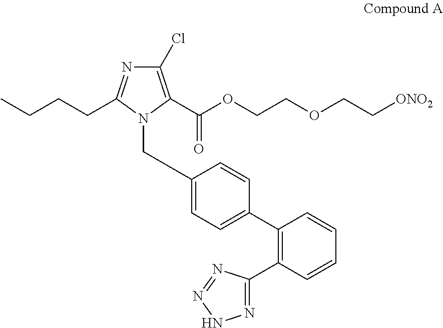

TABLE 2
Data

| | Δ SBP in SHR (mmHg) | | |
|---|---|---|---|
| Compound | 2-6 h | 12 h | 21-24 h |
| A (10 mpk) | −15 | −12 | −8 |
| Example 1 (10 mpk) | −19 | −14 | −11 |
| Example 2 (3 mpk) | −37 | −34 | −33 |

1-36. (canceled)

37. A compound having the general formula: 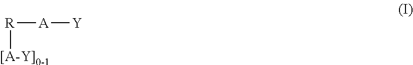
wherein R is selected from the group consisting o 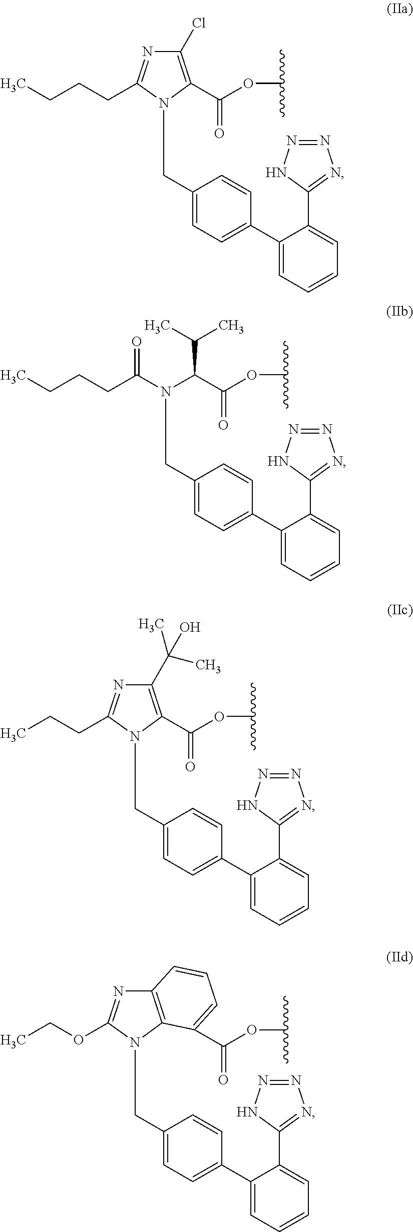 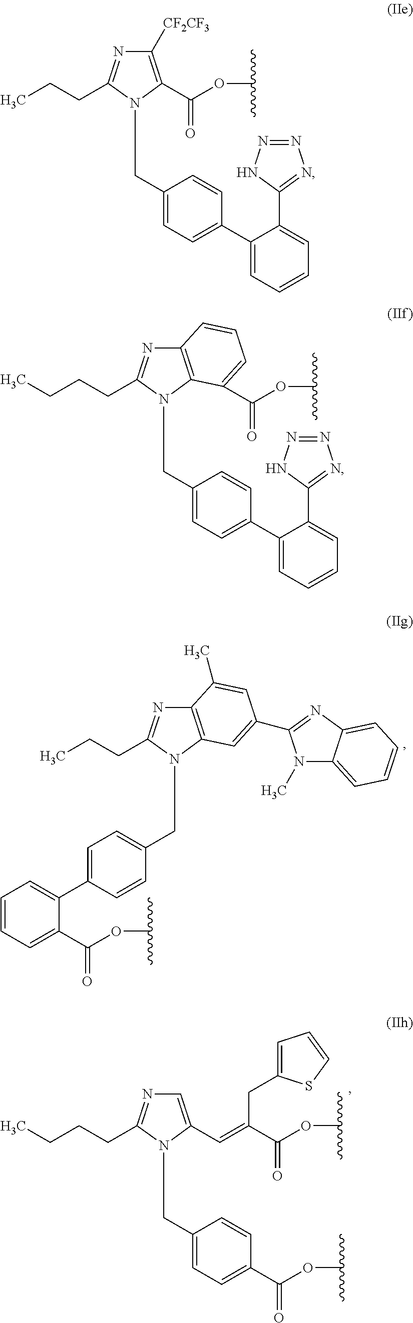
A is 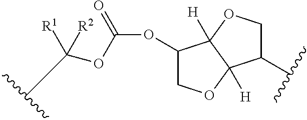
wherein R¹ and R² are independently selected from the group consisting of hydrogen and C₁₋₄ alkyl.
Y is X⁰—Z wherein X⁰ is selected from the group consisting of:
—O—, —O—CO—, —OCOO—, —OCONH— and —OSO₂—;
Z is selected from the group consisting of:
1) R⁰—CH₂—ONO₂,
wherein R⁰ is a straight or branched C₁-C₁₀ alkylene;
2) (CH₂)ₙR³,
3) (CH₂)ₙ—O—CH₂—R³,
wherein
R³ is —CH(ONO₂)R⁴;
R⁴ is —CH₃ or C₁₋₄ alkyl;
n is an integer from 1 to 6;
4) Y¹—R⁵,
wherein
R⁵ is —CH(ONO₂)CH(ONO₂)R⁶;
R⁶ is selected from —CH₃, —CH₂CH₃ and —CH(CH₃)₂;
Y¹ is —(CH₂)₁₋₄—(X)₀₋₁—(CH₂)₀₋₄, wherein X is —O— or —CR⁷R⁸—; and R⁷ and R⁸ are independently selected from the group consisting of hydrogen and C₁-C₄ alkyl;
5) Y¹—CH(ONO₂)CH₂(ONO₂)
wherein Y¹ is as above defined;
6) 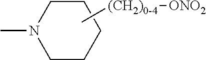
or a pharmaceutically acceptable salt thereof.

38. A compound of claim 37, wherein A is selected from the group consisting of: 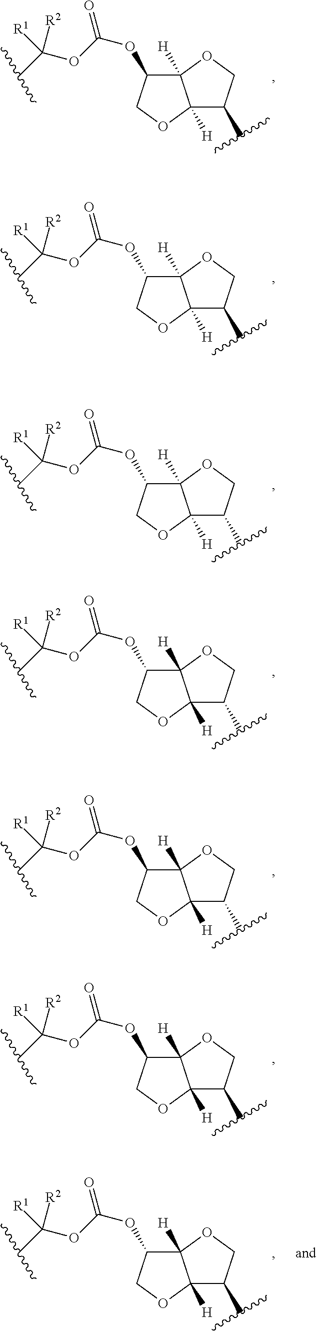 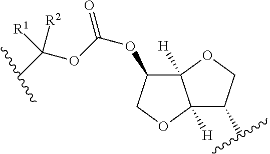
and all other variables are as previously defined.

39. A compound of claim 37, wherein R is (IId): 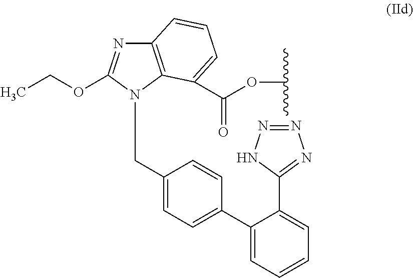
and A is selected from the group consisting of: 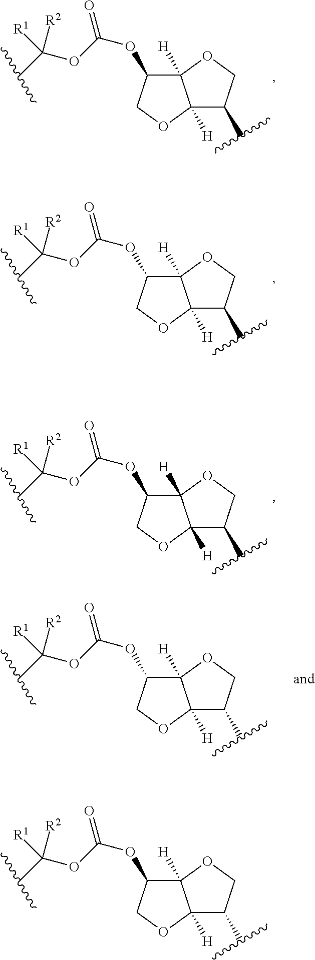
wherein R¹ and R² are CH₃.

40. A compound of claim 37, wherein R is (a (IId) or (IIg): 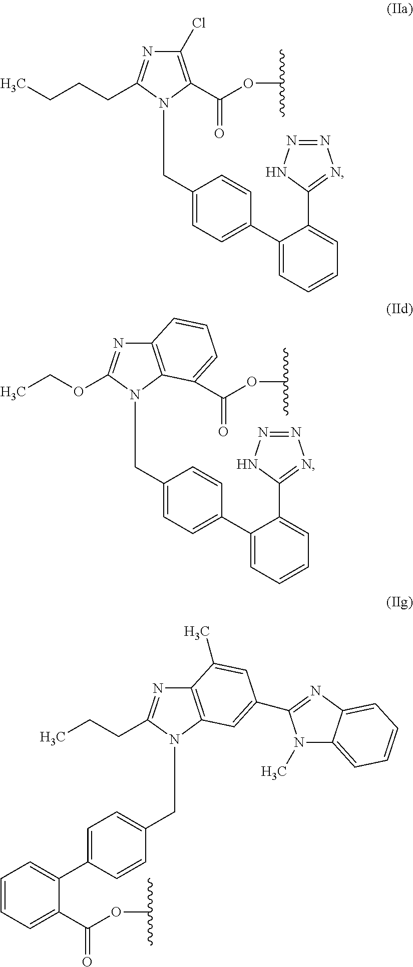
and A is selected from the group consisting of: 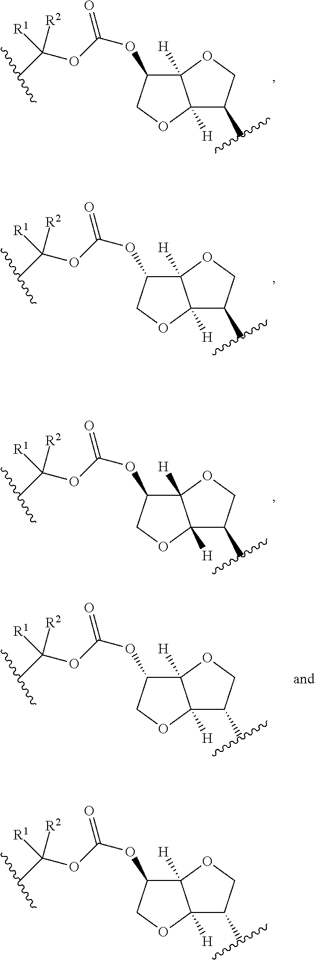
wherein R¹ is H and R² is CH₃.

41. A compound of claim 37, having the formula selected from the group consisting of 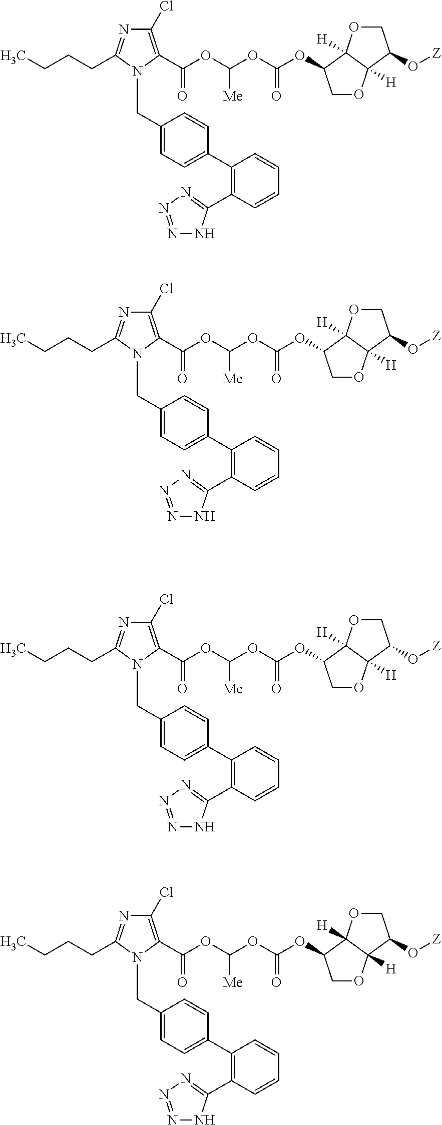
wherein Z is selected from the group consisting of
Z

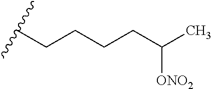

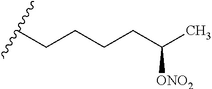

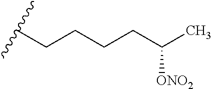

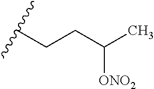

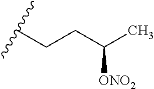

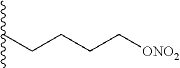

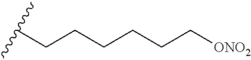

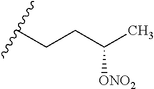

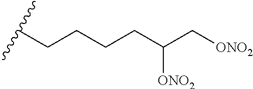

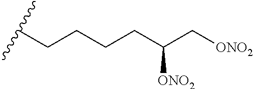

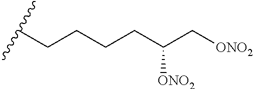

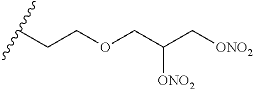

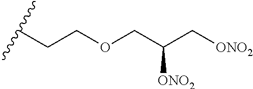

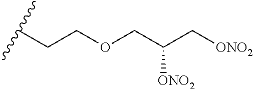

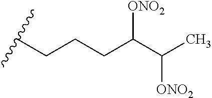

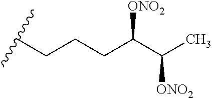

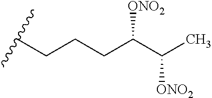

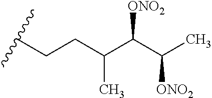

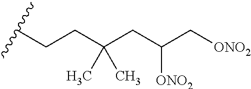

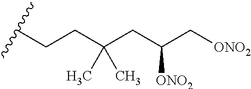

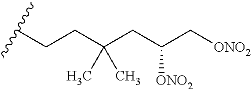

42. A compound of claim 37, having the formula selected from the group consisting of 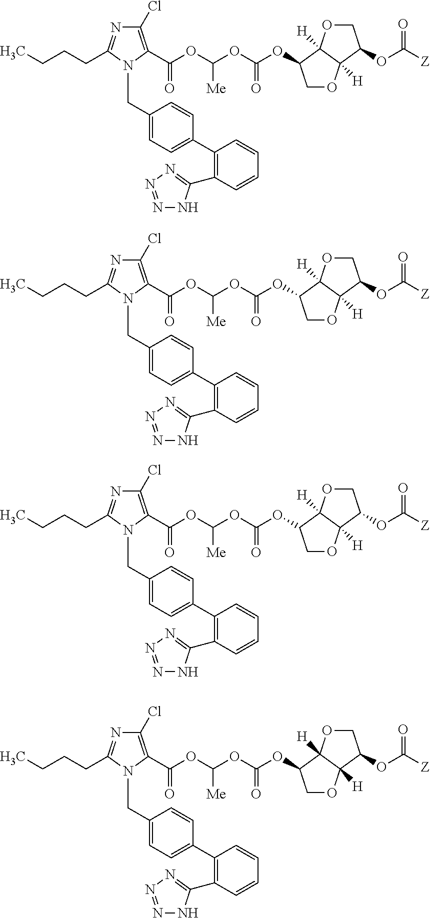
wherein Z is selected from the group consisting of:
Z

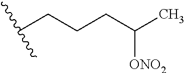

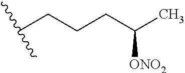

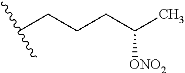

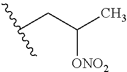

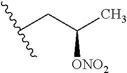

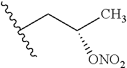

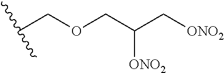

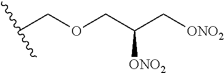

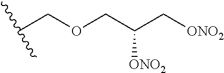

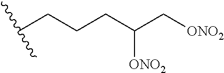

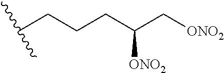

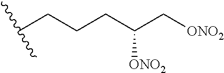

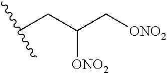

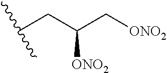

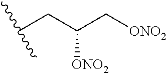

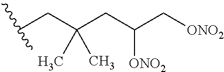

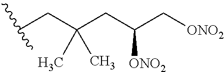

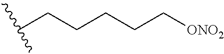

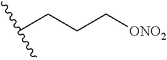

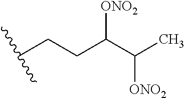

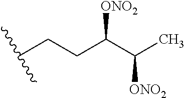

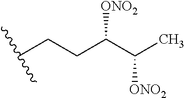

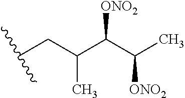

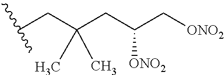

43. A compound of claim 37, having the formula selected from the group consisting of 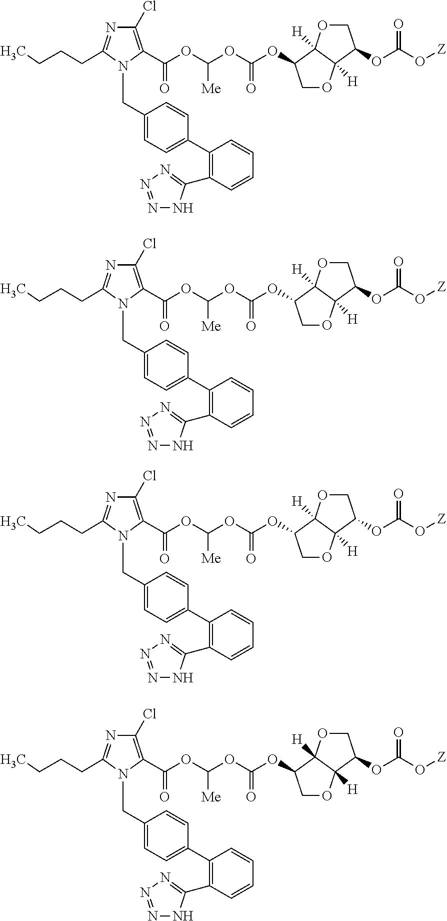
wherein Z is selected from the group consisting of:
Z

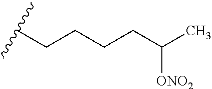

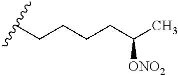

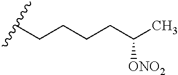

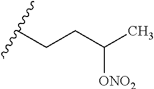

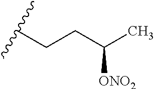

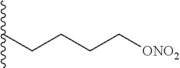

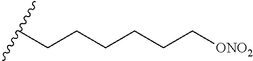

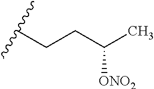

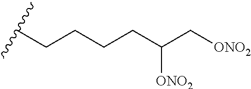

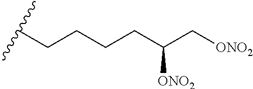

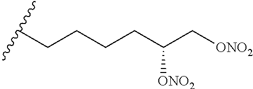

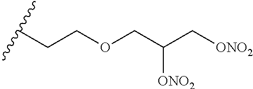

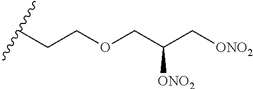

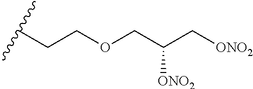

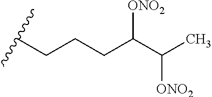

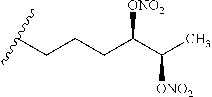

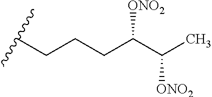

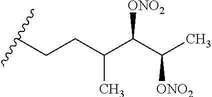

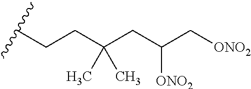

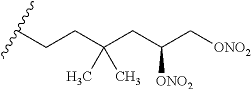

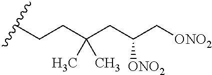

44. A compound of claim 37, having the formula selected from the group consisting of 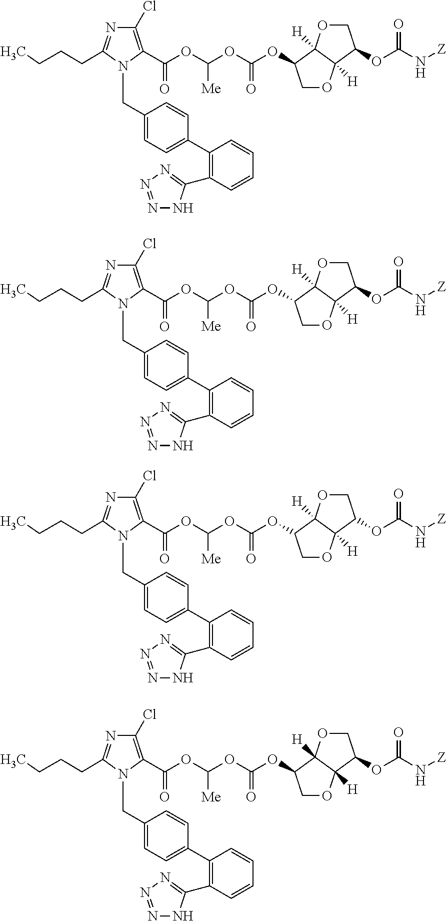
wherein Z is selected from the group consisting of:
Z

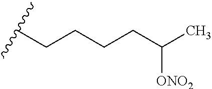

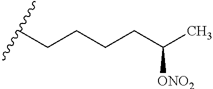

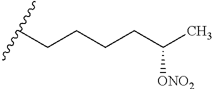

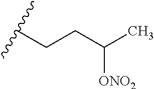

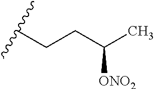

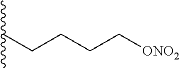

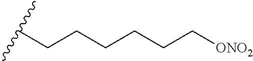

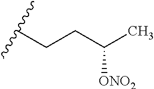

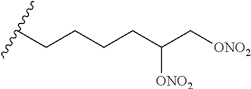

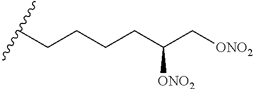

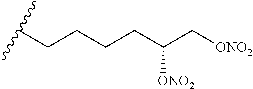

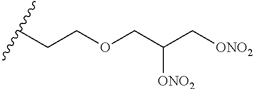

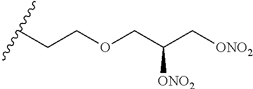

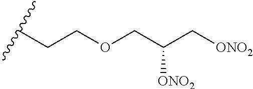

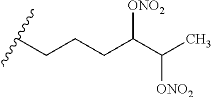

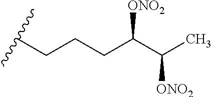

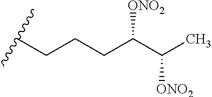

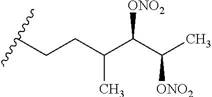

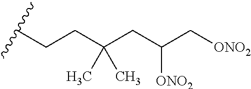

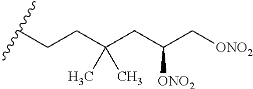

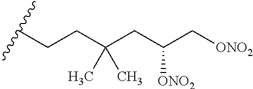

45. A compound of claim 37, having the formula selected from the group consisting of 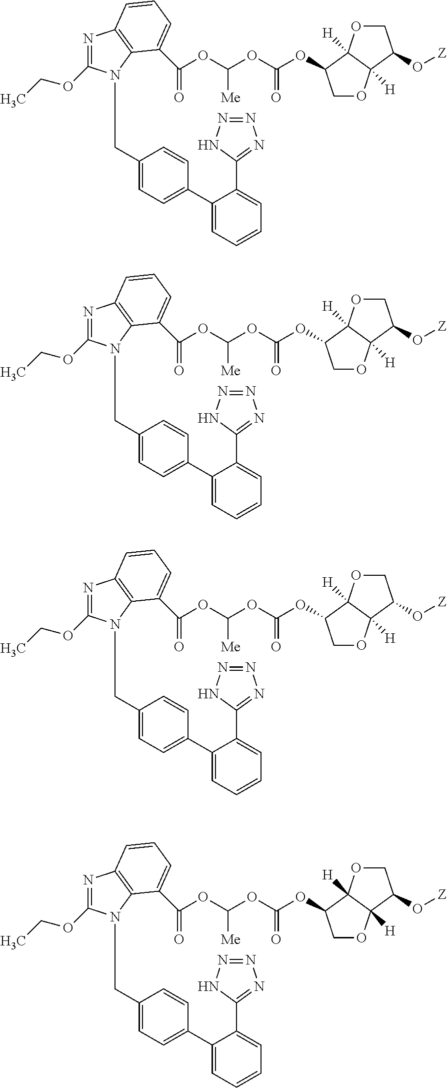
wherein Z is selected from the group consisting of:
Z

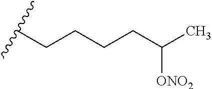

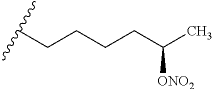

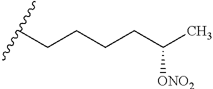

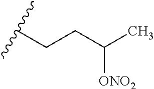

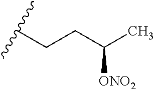

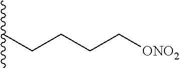

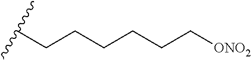

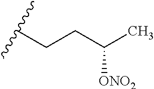

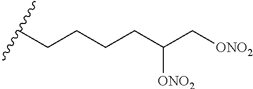

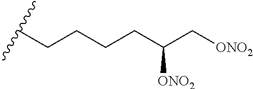

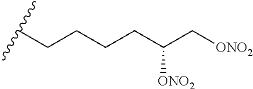

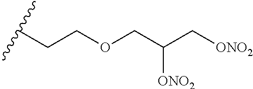

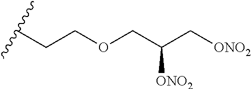

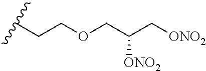

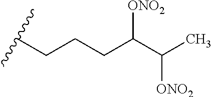

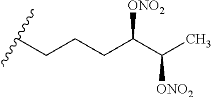

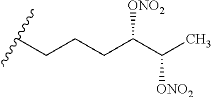

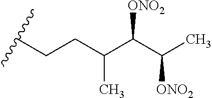

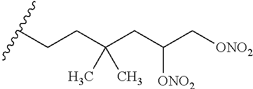

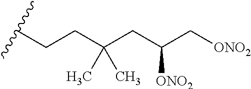

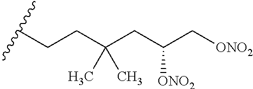

46. A compound of claim 37, having the formula selected from the group consisting of 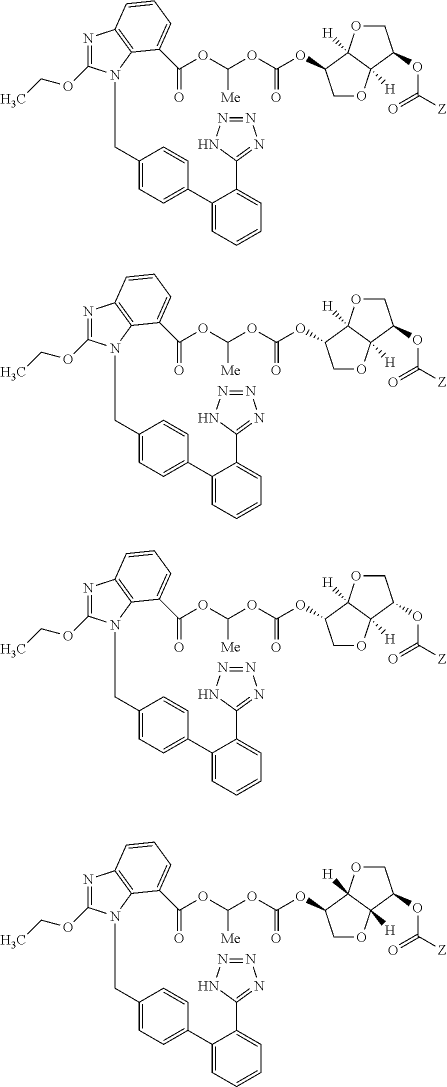
wherein Z is selected from the group consisting of:
Z

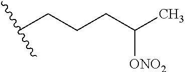

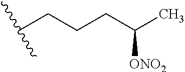

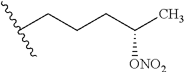

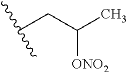

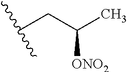

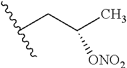

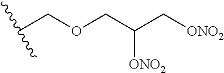

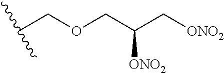

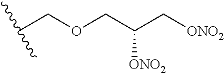

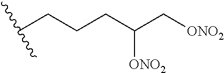

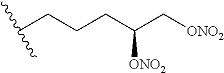

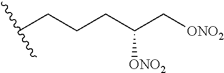

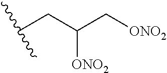

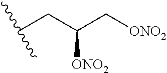

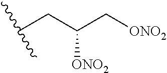

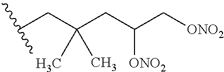

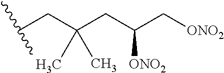

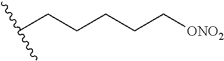

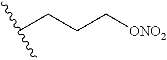

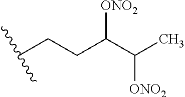

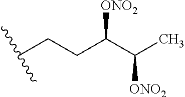

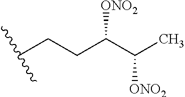

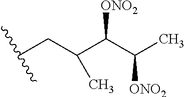

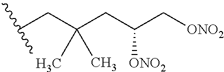

47. A compound of claim 37, having the formula selected from the group consisting of 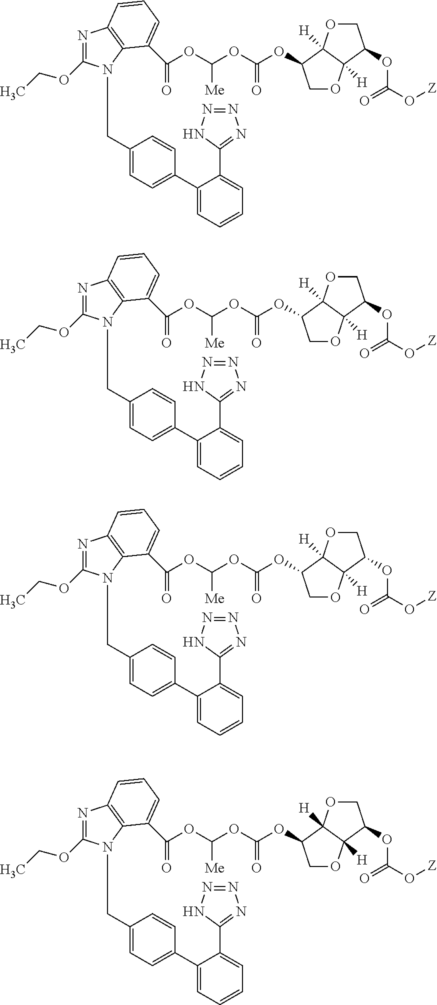
wherein Z is selected from the group consisting of:
Z

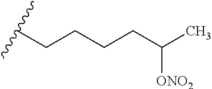

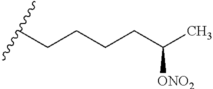

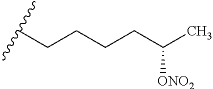

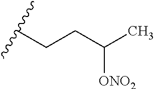

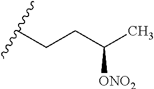

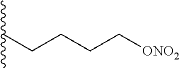

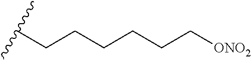

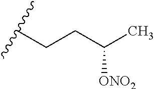

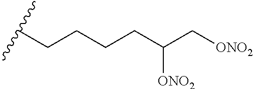

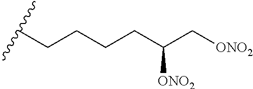

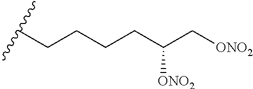

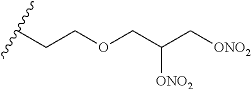

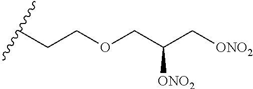

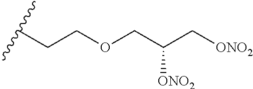

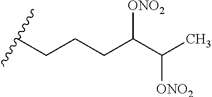

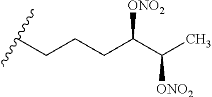

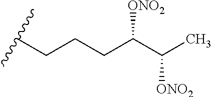

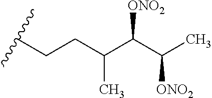

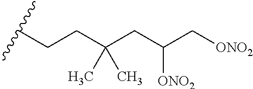

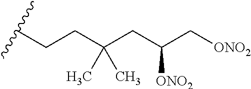

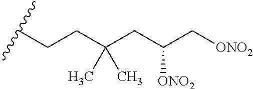

48. A compound of claim 37, having the formula selected from the group consisting of 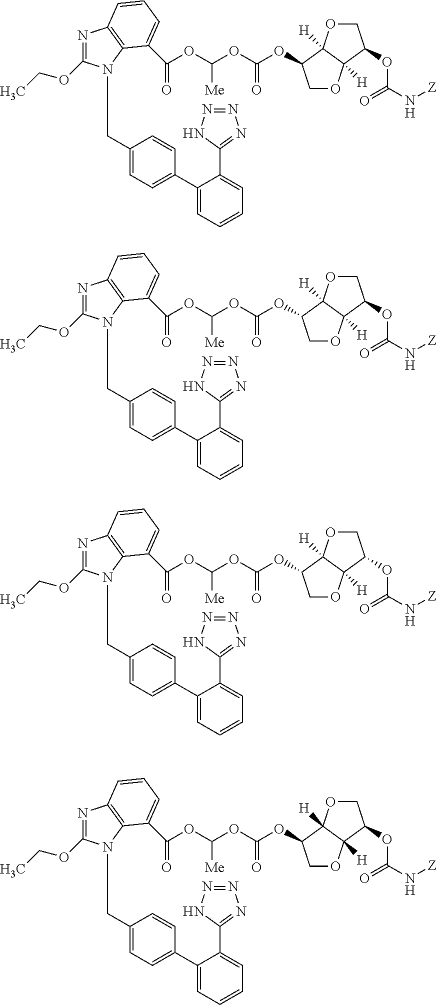
wherein Z is selected from the group consisting of:
Z

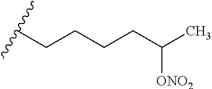

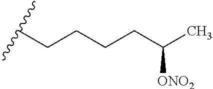

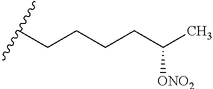

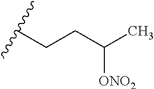

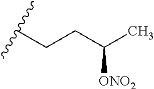

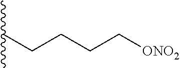

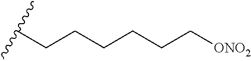

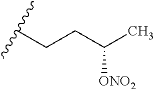

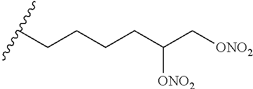

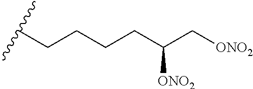

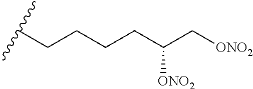

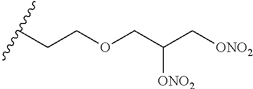

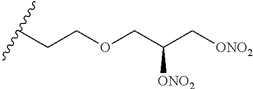

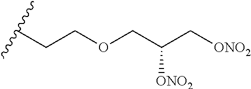

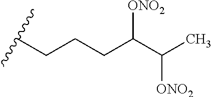

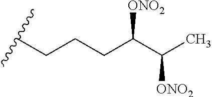

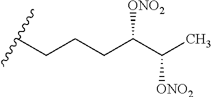

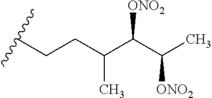

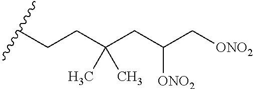

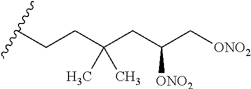

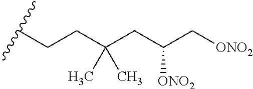

49. A pharmaceutical composition comprising a compound of claim 37 and a pharmaceutically acceptable carrier.

50. A pharmaceutical composition comprising a compound of claim 37, a diuretic, and a pharmaceutically acceptable carrier.

51. A method for treating hypertension in a patient which comprises administering to the patient a therapeutically effective amount of the composition of claim 49.
